(19) United States
(12) Patent Application Publication
(10) Pub. No.: US 2007/0021483 A1
(43) Pub. Date: Jan. 25, 2007

(54) Amidine derivatives for treating amyloidosis

(76) Inventors: Robert J. Chalifour, Ile Bizard (CA); Xianqi Kong, Dollard-des-Ormeaux (CA); Xinfu Wu, Laval (CA); Wenshuo Lu, San Diego, CA (US)

Correspondence Address: LAHIVE & COCKFIELD, LLP, ONE POST OFFICE SQUARE, BOSTON, MA, 02109-2127, US

(21) Appl. No.: 11/335,171

(22) Filed: Jan. 19, 2006

Related U.S. Application Data
(63) Continuation of application No. 10/731,463, filed on Dec. 5, 2003, now abandoned.
Application No. 10/731,463 is a continuation of application No. 10/234,643, filed on Sep. 3, 2002, now abandoned.
(60) Provisional application No. 60/316,761, filed on Aug. 31, 2001.
(60) Provisional application No. 60/387,001, filed on Jun. 7, 2002.

(51) Int. Cl. A61K 31/4184; A61K 31/155; C07D 403/02; C07C 257/12

(52) U.S. Cl. 514/394; 514/631; 564/225; 548/305.4

(57) ABSTRACT

The present invention relates to the use of amidine compounds in the treatment of amyloid-related diseases. In particular, the invention relates to a method of treating or preventing an amyloid-related disease in a subject comprising administering to the subject a therapeutic amount of an amidine compound. Among the compounds for use according to the invention are those according to the following Formula, such that, when administered, amyloid fibril formation, neurodegeneration, or cellular toxicity is reduced or inhibited:

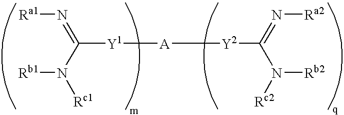

### RELATED APPLICATIONS

This application is a continuation of U.S. patent application Ser. No. 10/731,463, filed Dec. 5, 2003 (Atty. Docket No. NBI-105CN), which is a continuation of U.S. patent application Ser. No. 10/234,643, filed Sep. 3, 2002 (Atty. Docket No. NBI-105), and this application claims the priority of U.S. Provisional Patent Application Nos. 60/316,761, filed Aug. 31, 2001 (Atty. Docket No. NBI-105-1), and 60/387,001, filed Jun. 7, 2002 (Atty. Docket No. NBI-105-2), all of which are incorporated herein by reference.

### BACKGROUND OF THE INVENTION

Amyloidosis refers to a pathological condition characterized by the presence of amyloid fibers. Amyloid is a generic term referring to a group of diverse but specific protein deposits (intracellular or extracellular) which are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra.

Amyloid-related diseases can either be restricted to one organ or spread to several organs. The first instance is referred to as “localized amyloidosis” while the second is referred to as “systemic amyloidosis.”

Some amyloidotic diseases can be idiopathic, but most of these diseases appear as a complication of a previously existing disorder. For example, primary amyloidosis can appear without any other pathology or can follow plasma cell dyscrasia or multiple myeloma.

Secondary amyloidosis is usually seen associated with chronic infection (such as tuberculosis) or chronic inflammation (such as rheumatoid arthritis). A familial form of secondary amyloidosis is also seen in Familial Mediterranean Fever (FMF). This familial type of amyloidosis, as one of the other types of familial amyloidosis, is genetically inherited and is found in specific population groups. In these two types of amyloidosis, deposits are found in several organs and are thus considered systemic amyloid diseases.

Another type of systemic amyloidosis is found in long-term hemodialysis patients. In each of these cases, a different amyloidogenic protein is involved in amyloid deposition.

“Localized amyloidoses” are those that tend to involve a single organ system. Different amyloids are also characterized by the type of protein present in the deposit. For example, neurodegenerative diseases such as scrapie, bovine spongiform encephalitis, Creutzfeldt-Jakob disease, and the like are characterized by the appearance and accumulation of a protease-resistant form of a prion protein (referred to as AScr or PrP-27) in the central nervous system. Similarly, Alzheimer's disease, another neurodegenerative disorder, is characterized by neuritic plaques and neurofibrillary tangles. In this case, the plaque and blood vessel amyloid is formed by the deposition of fibrillary Aβ amyloid protein. Other diseases such as adult-onset diabetes (Type II diabetes) are characterized by the localized accumulation of amyloid in the pancreas.

Once these amyloids have formed, there is no known, widely accepted therapy or treatment which significantly dissolves amyloid deposits in situ.

Each amyloidogenic protein has the ability to organize into β-sheets and to form insoluble fibrils which may be deposited extracellularly or intracellularly. Each amyloidogenic protein, although different in amino acid sequence, has the same property of forming fibrils and binding to other elements such as proteoglycan, amyloid P and complement component. Moreover, each amyloidogenic protein has amino acid sequences which, although different, will show similarities such as regions with the ability to bind to the glycosaminoglycan (GAG) portion of proteoglycan (referred to as the GAG binding site) as well as other regions which will promote β-sheet formation.

In specific cases, amyloidotic fibrils, once deposited, can become toxic to the surrounding cells. For example, the Aβ fibrils organized as senile plaques have been shown to be associated with dead neuronal cells and microgliosis in patients with Alzheimer's disease. When tested in vitro, Aβ peptide was shown to be capable of triggering an activation process of microglia (brain macrophages), which would explain the presence of microgliosis and brain inflammation found in the brain of patients with Alzheimer's disease.

In another type of amyloidosis seen in patients with Type II diabetes, the amyloidogenic protein IAPP has been shown to induce β-islet cell toxicity in vitro. Hence, appearance of IAPP fibrils in the pancreas of Type II diabetic patients contributes to the loss of the β islet cells (Langerhans) and organ dysfunction.

People suffering from Alzheimer's disease develop a progressive dementia in adulthood, accompanied by three main structural changes in the brain: diffuse loss of neurons in multiple parts of the brain; accumulation of intracellular protein deposits termed neurofibrillary tangles; and accumulation of extracellular protein deposits termed amyloid or senile plaques, surrounded by misshapen nerve terminals (dystrophic neurites). A main constituent of these amyloid plaques is the amyloid-β peptide (Aβ), a 39-43 amino-acid protein that is produced through cleavage of the β-amyloid precursor protein (APP). Although symptomatic treatments exist for Alzheimer's disease, this disease cannot be prevented or cured at this time.

### SUMMARY OF THE INVENTION

The present invention relates to the use of amidine compounds in the treatment of amyloid-related diseases. In particular, the invention relates to a method of treating or preventing an amyloid-related disease in a subject comprising administering to the subject a therapeutic amount of an amidine compound. Among the compounds for use in the invention are those according to the following Formula, such that, when administered, amyloid fibril formation, neurodegeneration, or cellular toxicity is reduced or inhibited:

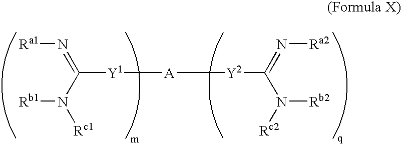

### BRIEF DESCRIPTION OF DRAWINGS

FIG. 1—Effect of pentamidine-type compounds on Aβ(1-40) assembly determined by ThT assay.

FIG. 2—Effect of pentamidine-like compounds on Aβ(1-40) assembly determined by ThT assay.

FIG. 3—Effect of amidine-type compounds on Aβ(1-40) assembly determined by ThT assay.

FIG. 4—Effect of pentamidine-type compounds on IAPP assembly determined by ThT assay.

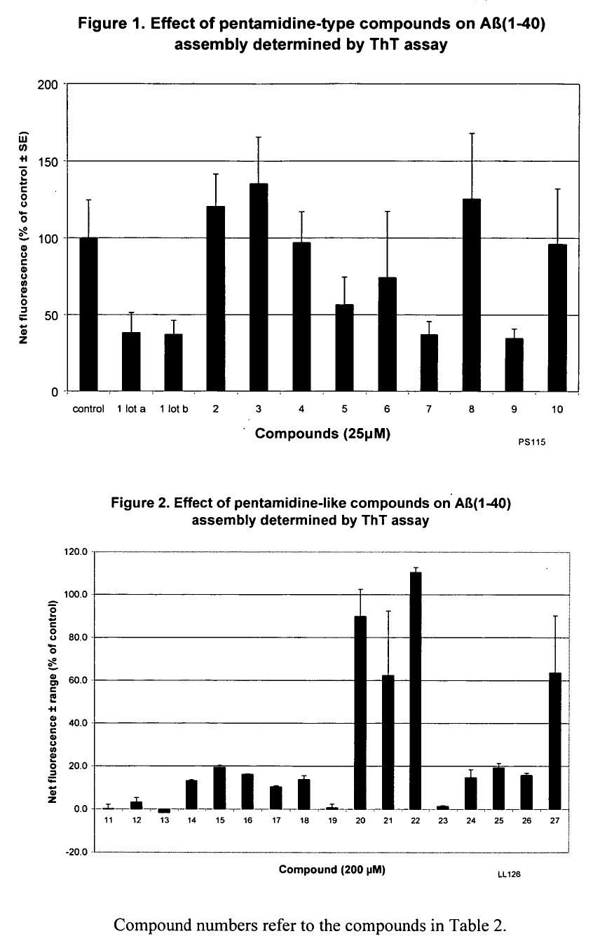

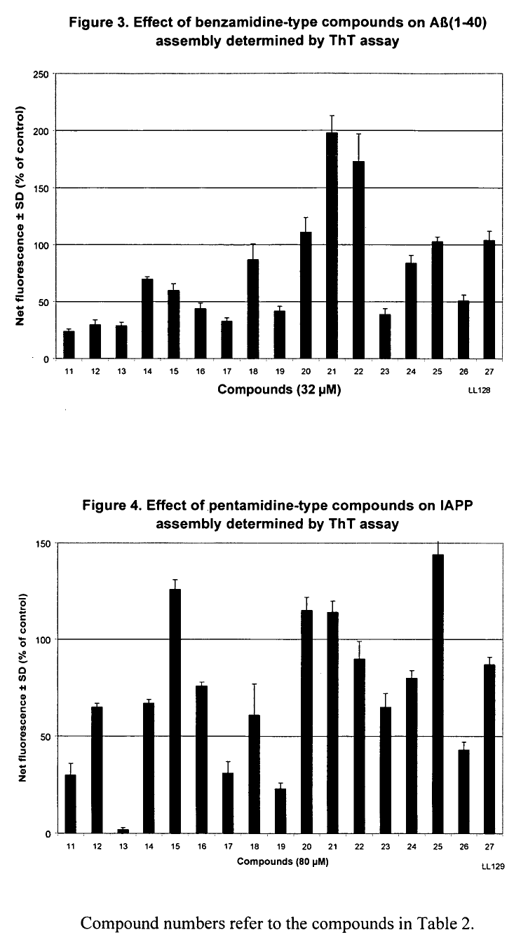

### DETAILED DESCRIPTION OF THE INVENTION

The present invention relates to the use of amidine compounds in the treatment of amyloid-related diseases.

Amyloid-Related Diseases

AA (Reactive) Amyloidosis

Generally, AA amyloidosis is a manifestation of a number of diseases that provoke a sustained acute phase response. Such diseases include chronic inflammatory disorders, chronic local or systemic microbial infections, and malignant neoplasms.

AA fibrils are generally composed of 8,000 Dalton fragments (AA peptide or protein) formed by proteolytic cleavage of serum amyloid A protein (ApoSAA), a circulating apolipoprotein which once secreted is complexed with HDL and which is synthesized in hepatocytes in response to such cytokines as IL-1, IL-6 and TNF. Deposition can be widespread in the body, with a preference for parenchymal organs. The spleen is usually a deposition site, and the kidneys may also be affected. Deposition is also common in the heart and gastrointestinal tract.

AA amyloid diseases include, but are not limited to inflammatory diseases, such as rheumatoid arthritis, juvenile chronic arthritis, ankylosing spondylitis, psoriasis, psoriatic arthropathy, Reiter's syndrome, Adult Still's disease, Behcet's syndrome, and Crohn's disease. AA deposits are also produced as a result of chronic microbial infections, such as leprosy, tuberculosis, bronchiectasis, decubitus ulcers, chronic pyelonephritis, osteomyelitis, and Whipple's disease. Certain malignant neoplasms can also result in AA fibril amyloid deposits. These include such conditions as Hodgkin's lymphoma, renal carcinoma, carcinomas of gut, lung and urogenital tract, basal cell carcinoma, and hairy cell leukemia.

AL Amyloidoses

AL amyloid deposition is generally associated with almost any dyscrasia of the B lymphocyte lineage, ranging from malignancy of plasma cells (multiple myeloma) to benign monoclonal gammopathy. At times, the presence of amyloid deposits may be a primary indicator of the underlying dyscrasia.

Fibrils of AL amyloid deposits are composed of monoclonal immunoglobulin light chains or fragments thereof. More specifically, the fragments are derived from the N-terminal region of the light chain (kappa or lambda) and contain all or part of the variable (V_{L}) domain thereof. Deposits generally occur in the mesenchymal tissues, causing peripheral and autonomic neuropathy, carpal tunnel syndrome, macroglossia, restrictive cardiomyopathy, arthropathy of large joints, immune dyscrasias, myelomas, as well as occult dyscrasias. However, it should be noted that almost any tissue, particularly visceral organs such as the heart, may be involved.

Hereditary Systemic Amyloidoses

There are many forms of hereditary systemic amyloidoses. Although they are relatively rare conditions, adult onset of symptoms and their inheritance patterns (usually autosomal dominant) lead to persistence of such disorders in the general population. Generally, the syndromes are attributable to point mutations in the precursor protein leading to production of variant amyloidogenic peptides or proteins. Table 1 summarizes the fibril composition of exemplary forms of these disorders.

TABLE 1

| Fibril Peptide/Protein | Genetic variant | Clinical Syndrome |
|---|---|---|
| Transthyretin and fragments | Met30, many others | Familial amyloid |
| (ATTR) | | polyneuropathy (FAP), (Mainly |
| | | peripheral nerves) |
| Transthyretin and fragments | Thr45, Ala60, Ser84, Met111, | Cardiac involvement |
| (ATTR) | Ile122 | predominant without neuropathy |
| N-terminal fragment of | Arg26 | Familial amyloid |
| Apolipoprotein A1 (apoAI) | | polyneuropathy (FAP), (mainly |
| | | peripheral nerves) |
| N-terminal fragment of | Arg26, Arg50, Arg 60, others | Ostertag-type, non-neuropathic |
| Apoliproprotein A1 (AapoAI) | | (predominantly visceral |
| | | involvement) |
| Lysozyme (Alys) | Thr56, His67 | Ostertag-type, non-neuropathic |
| | | (predominantly visceral |
| | | involvement) |
| Fibrogen ∀ chain fragment | Leu554, Val 526 | Cranial neuropathy with lattic |
| | | corneal dystrophy |
| Gelsolin fragment (Agel) | Asn187, Tyr187 | Cranial neuropathy with lattice |
| | | corneal dystrophy |
| Cystatin C fragment | Glu68 | Hereditary cerebral hemorrhage |
| | | (cerebral amyloid angiopathy) — |
| | | Icelandic type |
| β-amyloid protein (Aβ) derived | Gln693 | Hereditary cerebral hemorrhage |
| from Amyloid Precursor Protein | | (cerebral amyloid angiopathy) - |
| (APP) | | Dutch type |
| β-amyloid protein (Aβ) derived | Ile717, Phe717, Gly717 | Familial Alzheimer's Disease |
| from Amyloid Precursor Protein | | |
| (APP) | | |
| β-amyloid protein (Aβ) derived | Asn670, Leu671 | Familial Dementia - probably |
| from Amyloid Precursor Protein | | Alzheimer's Disease |
| (APP) | | |
| Prion Protein (PrP) derived from | Leu102, Val167, Asn178, | Familial Creutzfeldt - Jakob |
| Prp precursor protein | Lys200 | disease; Gerstmann-Straussler- |
| 51-91 insert | | Scheinker syndrome (hereditary |
| | | spongiform encephalopathies, |
| | | prion diseases) |
| AA derived from Serum | | Familial Mediterranean fever, |
| amyloid A protein (ApoSAA) | | predominant renal involvement |
| | | (autosomal recessive) |
| AA derived from Serum | | Muckle-Well's syndrome, |
| amyloid A protein (ApoSAA) | | nephropathy, deafness, urticaria, |
| | | limb pain |
| Unknown | | Cardiomyopathy with persistent |
| | | atrial standstill |
| Unknown | | Cutaneous deposits (bullous, |
| | | papular, pustulodermal) |

Data derived from Tan SY, Pepys MB. Amyloidosis. Histopathology, 25(5), 403-414 (Nov 1994).

The data provided in Table 1 are exemplary and are not intended to limit the scope of the invention. For example, more than 40 separate point mutations in the transthyretin gene have been described, all of which give rise to clinically similar forms of familial amyloid polyneuropathy.

Transthyretin (TTR) is a 14 kiloDalton protein that is also sometimes referred to as prealbumin. It is produced by the liver and choroid plexus, and it functions in transporting thyroid hormones and vitamin A. At least 50 variant forms of the protein, each characterized by a single amino acid change, are responsible for various forms of familial amyloid polyneuropathy. For example, substitution of proline for leucine at position 55 results in a particularly progressive form of neuropathy; substitution of methionine for leucine at position 111 resulted in a severe cardiopathy in Danish patients.

Amyloid deposits isolated from heart tissue of patients with systemic amyloidosis have revealed that the deposits are composed of a heterogeneous mixture of TTR and fragments thereof, collectively referred to as ATTR, the full length sequences of which have been characterized. ATTR fibril components can be extracted from such plaques and their structure and sequence determined according to the methods known in the art (e.g., Gustavsson, A., et al., Laboratory Invest. 73: 703-708, 1995; Kametani, F., et al., Biochem. Biophys. Res. Commun. 125: 622-628, 1984; Pras, M., et al., PNAS 80: 539-42, 1983).

Persons having point mutations in the molecule apolipoprotein Al (e.g., Gly→Arg26; Trp→Arg50; Leu→Arg60) exhibit a form of amyloidosis (“Östertag type”) characterized by deposits of the protein apolipoprotein AI or fragments thereof (AApoAI). These patients have low levels of high density lipoprotein (HDL) and present with a peripheral neuropathy or renal failure.

A mutation in the alpha chain of the enzyme lysozyme (e.g., Ile→Thr56 or Asp→His57) is the basis of another form of Östertag-type non-neuropathic hereditary amyloid reported in English families. Here, fibrils of the mutant lysozyme protein (Alys) are deposited, and patients generally exhibit impaired renal function. This protein, unlike most of the fibril-forming proteins described herein, is usually present in whole (unfragmented) form (Benson, M. D., et al. CIBA Fdn. Symp. 199: 104-131, 1996).

β-amyloid peptide (Aβ) is a 39-43 amino acid peptide derived by proteolysis from a large protein known as Beta Amyloid Precursor protein (βAPP). Mutations in βAPP result in familial forms of Alzheimer's disease, Down's syndrome or senile dementia, characterized by cerebral deposition of plaques composed of Aβ fibrils and other components, which are described in further detail below. Known mutations in APP associated with Alzheimer's disease occur proximate to the cleavage sites of β or gamma-secretase, or within Aβ. For example, position 717 is proximate to the site of gamma-secretase cleavage of APP in its processing to Aβ, and positions 670/671 are proximate to the site of P-secretase cleavage. Mutations at any of these residues may result in Alzheimer's disease, presumably by causing an increase in the amount of the 42/43 amino acid form of Aβ generated from APP.

The structure and sequence of Aβ peptides of various lengths are well known in the art. Such peptides can be made according to methods known in the art (e.g., Glenner and Wong, Biochem Biophys. Res. Comm. 129: 885-890, 1984; Glenner and Wong, Biochem Biophys. Res. Comm. 122: 113 1-1135, 1984). In addition, various forms of the peptides are commercially available.

As used herein, the term “β amyloid” or “amyloid-β” refer to amyloid D proteins or peptides, amyloid β precursor proteins or peptides, intermediates, and modifications and fragments thereof, unless otherwise specifically indicated. In particular, “Aβ” refers to any peptide produced by proteolytic processing of the APP gene product, especially peptides which are associated with amyloid pathologies, including Aβ₁₋₃₉, Aβ₁₋₄₀, Aβ₁₄₁, Aβ₁₋₄₂, and Aβ₁₋₄₃.

For convenience of nomenclature, “Aβ₁₋₄₂” may be referred to herein as “Aβ(1-42) or simply as “Aβ₄₂” or “Aβ42” (and likewise for any other amyloid peptides discussed herein). As used herein, the terms “β amyloid,” “amyloid-β,” and “Aβ” are synonymous.

Unless otherwise specified, the term “amyloid” refers to amyloidogenic proteins, peptides, or fragments thereof which can be soluble (e.g., monomeric or oligomeric) or insoluble (e.g., having fibrillary structure or in amyloid plaque).

Gelsolin is a calcium binding protein that binds to fragments and actin filaments. Mutations at position 187 (e.g., Asp→Asn; Asp→Tyr) of the protein result in a form of hereditary systemic amyloidosis, usually found in patients from Finland, as well as persons of Dutch or Japanese origin. In afflicted individuals, fibrils formed from gelsolin fragments (Agel), usually consist of amino acids 173-243 (68 kDa carboxyterminal fragment) and are deposited in blood vessels and basement membranes, resulting in corneal dystrophy and cranial neuropathy which progresses to peripheral neuropathy, dystrophic skin changes and deposition in other organs. (Kangas, H., et al. Human Mol. Genet. 5(9): 1237-1243, 1996).

Other mutated proteins, such as mutant alpha chain of fibrinogen (AfibA) and mutant cystatin C (Acys) also form fibrils and produce characteristic hereditary disorders. AfibA fibrils form deposits characteristic of a nonneuropathic hereditary amyloid with renal disease; Acys deposits are characteristic of a hereditary cerebral amyloid angiopathy reported in Iceland (Isselbacher, Harrison's Principles of Internal Medicine, McGraw-Hill, San Francisco, 1995; Benson, et al.). In at least some cases, patients with cerebral amyloid angiopathy (CAA) have been shown to have amyloid fibrils containing a non-mutant form of cystatin C in conjunction with amyloid beta protein (Nagai, A., et al. Molec. Chem. Neuropathol. 33: 63-78, 1998).

Certain forms of prion disease are now considered to be heritable, accounting for up to 15% of cases, which were previously thought to be predominantly infectious in nature. (Baldwin, et al., in Research Advances in Alzheimer's Disease and Related Disorders, John Wiley and Sons, New York, 1995). In such prion disorders, patients develop plaques composed of abnormal isoforms of the normal prion protein (PrP^{Sc}).

A predominant mutant isoform, PrP^{Sc}, also referred to as AScr, differs from the normal cellular protein in its resistance to protease degradation, insolubility after detergent extraction, deposition in secondary lysosomes, post-translational synthesis, and high β-pleated sheet content. Genetic linkage has been established for at least five mutations resulting in Creutzfeldt-Jacob disease (CJD), Gerstmann-Straussler-Scheinker syndrome (GSS), and fatal familial insomnia (FFI). (Baldwin, supra) Methods for extracting fibril peptides from scrapie fibrils, determining sequences and making such peptides are known in the art (e.g., Beekes, M., et al. J. Gen. Virol. 76: 2567-76, 1995).

For example, one form of GSS has been linked to a PrP mutation at codon 102, while telencephalic GSS segregates with a mutation at codon 117. Mutations at codons 198 and 217 result in a form of GSS in which neuritic plaques characteristic of Alzheimer's disease contain PrP instead of Aβ peptide. Certain forms of familial CJD have been associated with mutations at codons 200 and 210; mutations at codons 129 and 178 have been found in both familial CJD and FFI. (Baldwin, supra).

Senile Systemic Amyloidosis

Amyloid deposition, either systemic or focal, increases with age. For example, fibrils of wild type transthyretin (TTR) are commonly found in the heart tissue of elderly individuals. These may be asymptomatic, clinically silent, or may result in heart failure. Asymptomatic fibrillar focal deposits may also occur in the brain (Aβ), corpora amylacea of the prostate (Aβ₂ microglobulin), joints and seminal vesicles.

Cerebral Amyloidosis

Local deposition of amyloid is common in the brain, particularly in elderly individuals. The most frequent type of amyloid in the brain is composed primarily of Aβ peptide fibrils, resulting in dementia or sporadic (non-hereditary) Alzheimer's disease. In fact, the incidence of sporadic Alzheimer's disease greatly exceeds forms shown to be hereditary. Fibril peptides forming these plaques are very similar to those described above, with reference to hereditary forms of Alzheimer's disease (AD).

Cerebral amyloid angiopathy (CAA) refers to the specific deposition of amyloid fibrils in the walls of leptomingeal and cortical arteries, arterioles and in capillaries and veins. It is commonly associated with Alzheimer's disease, Down's syndrome and normal aging, as well as with a variety of familial conditions related to stroke or dementia (see Frangione et al., Amyloid: J. Protein Folding Disord. 8, Suppl. 1, 36-42 (2001)). CAA can occur sporadically or be hereditary. Multiple mutation sites in either Aβ or the APP gene have been identified and are clinically associated with either dementia or cerebral hemorrhage. Exemplary CAA disorders include, but are not limited to, hereditary cerebral hemorrhage with amyloidosis of Icelandic type (HCHWA-I); the Dutch variant of HCHWA (HCHWA-D; a mutation in Aβ); the Flemish mutation of Aβ; the Arctic mutation of Aβ; the Italian mutation of Aβ; the Iowa mutation of Aβ; familial British dementia; and familial Danish dementia.

Dialysis-Related Amyloidosis

Plaques composed of O₂ microglobulin (Aβ₂M) fibrils commonly develop in patients receiving long term hemodialysis or peritoneal dialysis. β₂ microglobulin is a 11.8 kiloDalton polypeptide and is the light chain of Class I MHC antigens, which are present on all nucleated cells. Under normal circumstances, it is continuously shed from cell membranes and is normally filtered by the kidney. Failure of clearance, such as in the case of impaired renal function, leads to deposition in the kidney and other sites (primarily in collagen-rich tissues of the joints). Unlike other fibril proteins, Aβ₂M molecules are generally present in unfragmented form in the fibrils. (Benson, supra).

Islet Amyloid Polypeptide and Diabetes

Islet hyalinosis (amyloid deposition) was first described over a century ago as the presence of fibrous protein aggregates in the pancreas of patients with severe hyperglycemia (Opie, E L., J Exp. Med. 5: 397-428, 1990). Today, islet amyloid, composed predominantly of islet amyloid polypeptide (IAPP), or amylin, is a characteristic histopathological marker in over 90% of all cases of Type II diabetes (also known as Non-Insulin Dependent Diabetes, or NIDDM). These fibrillar accumulations result from the aggregation of the islet amyloid polypeptide (IAPP) or amylin, which is a 37 amino acid peptide, derived from a larger precursor peptide, called pro-IAPP.

IAPP co-localizes and is co-secreted with insulin in response to β-cell secretagogues. This pathological feature is not associated with insulin-dependent (Type I) diabetes and is a unifying characteristic for the heterogeneous clinical phenotypes diagnosed as NIDDM (Type II diabetes).

Longitudinal studies in cats and immunocytochemical investigations in monkeys have shown that a progressive increase in islet amyloid is associated with a dramatic decrease in the population of insulin-secreting β-cells and increased severity of the disease. More recently, transgenic studies have strengthened the relationship between IAPP plaque formation and β-cell dysfunction, indicating that amyloid deposition is a principal factor in Type-II diabetes.

IAPP has also been shown to induce P-islet cell toxicity in vitro, indicating that appearance of IAPP fibrils in the pancreas of Type II or Type I diabetic patients (post-transplantation) could contribute to the loss of the P islet cells (Langerhans) and organ dysfunction. In patients with Type-IT diabetes, the accumulation of pancreatic IAPP leads to a buildup of IAPP-amyloid as insoluble fibrous deposits which eventually replace the insulin-producing β cells of the islet resulting in β cell depletion and failure (Westermark, P., Grimelius, L., Acta Path. Microbiol. Scand., sect. A. 81: 291-300, 1973; de Koning, E J P., et al., Diabetologia 36: 378-384, 1993; and Lorenzo, A., et al., Nature 368: 756-760, 1994).

Diseases caused by the death or malfunctioning of a particular type or types of cells can be treated by transplanting into the patient healthy cells of the relevant type of cell. This approach has been used for Type I diabetes patients. Often pancreatic islet cells are cultured in vitro prior to transplantation to increase their numbers, to allow them to recover after the isolation procedure or to reduce their immunogenicity. However, in many instances islet cell transplantation is unsuccessful, due to death of the transplanted cells. One reason for this poor success rate is IAPP, which can form fibrils and become toxic to the cells in vitro. In addition, IAPP fibrils are likely to continue to grow after the cells are transplanted and cause death or dysfunction of the cells. This may occur even when the cells are from a healthy donor and the patient receiving the transplant does not have a disease that is characterized by the presence of fibrils. For example, compounds of the present invention may also be used in preparing tissues or cells for transplantation according to the methods described in International Patent Application (PCT) number WO 01/03,680.

Hormone-Derived Amyloidoses

Endocrine organs may harbor amyloid deposits, particularly in aged individuals. Hormone-secreting tumors may also contain hormone-derived amyloid plaques, the fibrils of which are made up of polypeptide hormones such as calcitonin (medullary carcinoma of the thyroid), islet amyloid polypeptide (amylin; occurring in most patients with Type II diabetes), and atrial natriuretic peptide (isolated atrial amyloidosis). Sequences and structures of these proteins are well known in the art.

Miscellaneous Amyloidoses

There are a variety of other forms of amyloid disease that are normally manifest as localized deposits of amyloid. In general, these diseases are probably the result of the localized production or lack of catabolism of specific fibril precursors or a predisposition of a particular tissue (such as the joint) for fibril deposition. Examples of such idiopathic deposition include nodular AL amyloid, cutaneous amyloid, endocrine amyloid, and tumor-related amyloid.

The compounds of the invention may be administered therapeutically or prophylactically to treat diseases associated with amyloid-β fibril formation, aggregation or deposition. The compounds of the invention may act to ameliorate the course of an amyloid-β related disease using any of the following mechanisms (this list is meant to be illustrative and not limiting): slowing the rate of amyloid-β fibril formation or deposition; lessening the degree of amyloid-β deposition; inhibiting, reducing, or preventing amyloid-β fibril formation; inhibiting neurodegeneration or cellular toxicity induced by amyloid-p; inhibiting amyloid-β induced inflammation; or enhancing the clearance of amyloid-β from the brain.

Compounds of the invention may be effective in controlling amyloid-β deposition either following their entry into the brain (following penetration of the blood brain barrier) or from the periphery. When acting from the periphery, a compound may alter the equilibrium of Aβ between the brain and the plasma so as to favor the exit of Aβ from the brain. An increase in the exit of Aβ from the brain would result in a decrease in Aβ brain concentration and therefore favor a decrease in Aβ deposition. Alternatively, compounds that penetrate the brain could control deposition by acting directly on brain Aβ, e.g., by maintaining it in a non-fibrillar form or favoring its clearance from the brain.

In a preferred embodiment, the method is used to treat Alzheimer's disease (e.g., sporadic or familial AD). The method can also be used prophylactically or therapeutically to treat other clinical occurrences of amyloid-β deposition, such as in Down's syndrome individuals and in patients with cerebral amyloid angiopathy (“CAA”) or hereditary cerebral hemorrhage.

Additionally, abnormal accumulation of APP and of amyloid-β protein in muscle fibers has been implicated in the pathology of sporadic inclusion body myositis (IBM) (Askanas, V., et al. (1996) Proc. Natl. Acad. Sci. USA 93: 1314-1319; Askanas, V. et al. (1995) Current Opinion in Rheumatology 7: 486-496). Accordingly, the compounds of the invention can be used prophylactically or therapeutically in the treatment of disorders in which amyloid-beta protein is abnormally deposited at non-neurological locations, such as treatment of IBM by delivery of the compounds to muscle fibers.

The present invention therefore relates to the use of amidine compounds in the prevention or treatment of amyloid-related diseases, including, inter alia, Alzheimer's disease, cerebral amyloid angiopathy, inclusion body myositis, Down's syndrome, and type II diabetes.

Preferred compounds of the invention have at least two amidine moieties (preferably arylamidines, more preferably benzamidines).

In one particular embodiment, the present invention relates to the novel use of amidine compounds in the prevention or treatment of amyloid-related diseases, such as those disclosed in U.S. Pat. Nos. 5,428,051, 4,963,589, 5,202,320, 5,935,982, 5,521,189, 5,686,456, 5,627,184, 5,622,955, 5,606,058, 5,668,167, 5,667,975, 6,025,398, 6,214,883, 5,817,687, 5,792,782, 5,939,440, 6,017,941, 5,972,969, 6,046,226, 6,294,565 (B1), 6,156,779, 6,326,395, 6,008,247, 6,127,554, 6,172,104, 4,940,723, 5,594,138, 5,602,172, 5,206,236, 5,843,980, 4,933,347, 5,668,166, 5,817,686, 5,723,495, 4,619,942, 5,792,782, 5,639,755, 5,643,935, and 5,578,631, each of which are hereby incorporated herein by reference in their entirety.

In another embodiment, the invention relates to a method of treating or preventing an amyloid-related disease in a subject (preferably a human) comprising administering to the subject a therapeutic amount of a compound according to the following Formula, such that amyloid fibril formation or deposition, neurodegeneration, or cellular toxicity is reduced or inhibited. In another embodiment, the invention relates to a method of treating or preventing an amyloid-related disease in a subject (preferably a human) comprising administering to the subject a therapeutic amount of a compound according to the following Formula, such that cognitive function is stabilized or further deterioration in cognitive function is prevented, slowed, or stopped in patients with brain amyloidosis, e.g., Alzheimer's disease or cerebral amyloid angiopathy:

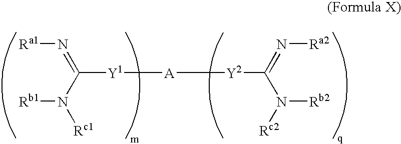

wherein each of R^{a1}, R^{b1}, R^{c1}, R^{a2}, R^{b2}, and R^{c2} is independently a hydrogen, a Z group, or R^{a1} and R^{b1} or R^{a2} and R^{b2} are both taken together along with the nitrogen atoms to which they are bound to form a ring structure;

each of Y¹ and Y² is independently a direct bond or a linking moiety;

m and q are each independently an integer selected from zero to five inclusive, such that 1≦m+q≦5, or in another embodiment, 2≦m+q≦5, or in another embodiment 1≦m+q≦10, or in another embodiment, 2≦m+q≦0; and

The A group is a carrier moiety selected from substituted or unsubstituted aliphatic and aromatic groups, and combinations thereof; preferably such that the Y¹ and Y² moieties are bonded to an aromatic group.

The A group preferably is a divalent group (i.e., m+q=2) such as an alkylene group (i.e., —(CH₂)ₖ— and substituted analogs thereof (including groups in which a —CH₂— moiety is substituted by an oxygen atom), where k is 1 to 12 (preferably 6 to 9, more preferably 7 to 9), an alkenylene group (preferably 2 to 12 carbon atoms, more preferably 6 to 9 carbon atoms, including groups with more than one double bond), an alkynylene group (preferably 2 to 12 carbon atoms, more preferably 6 to 9 carbon atoms, including groups with more than one triple bond), an alkoxyalkylene group, an alkylaminoalkylene group, a thioalkoxyalkylene group, an arylenedialkylene group, a heteroarylenedialkylene group, an arylene group, a heteroarylene group, an oligoethereal group such as an oligo(alkyleneoxide) group, or an arylene-di(oligoalkyleneoxide) group, each of which may be substituted (with a Z group as defined below, e.g., a hydroxyalkylene group) or unsubstituted.

The A group also includes the corresponding moieties of the Formulae I-IV herein.

In preferred aspects of the invention, the invention relates to a method of treating or preventing an amyloid-related disease in a subject (preferably a human) comprising administering to the subject a therapeutic amount of a compound according to one of the following Formulae, such that amyloid fibril formation or deposition, neurodegeneration, or cellular toxicity is reduced or inhibited. In another embodiment, the invention relates to a method of treating or preventing an amyloid-related disease in a subject (preferably a human) comprising administering to the subject a therapeutic amount of a compound according to one of the following Formulae, such that cognitive function is stabilized or further deterioration in cognitive function is prevented, slowed, or stopped in patients with brain amyloidosis, e.g., Alzheimer's disease or cerebral amyloid angiopathy:

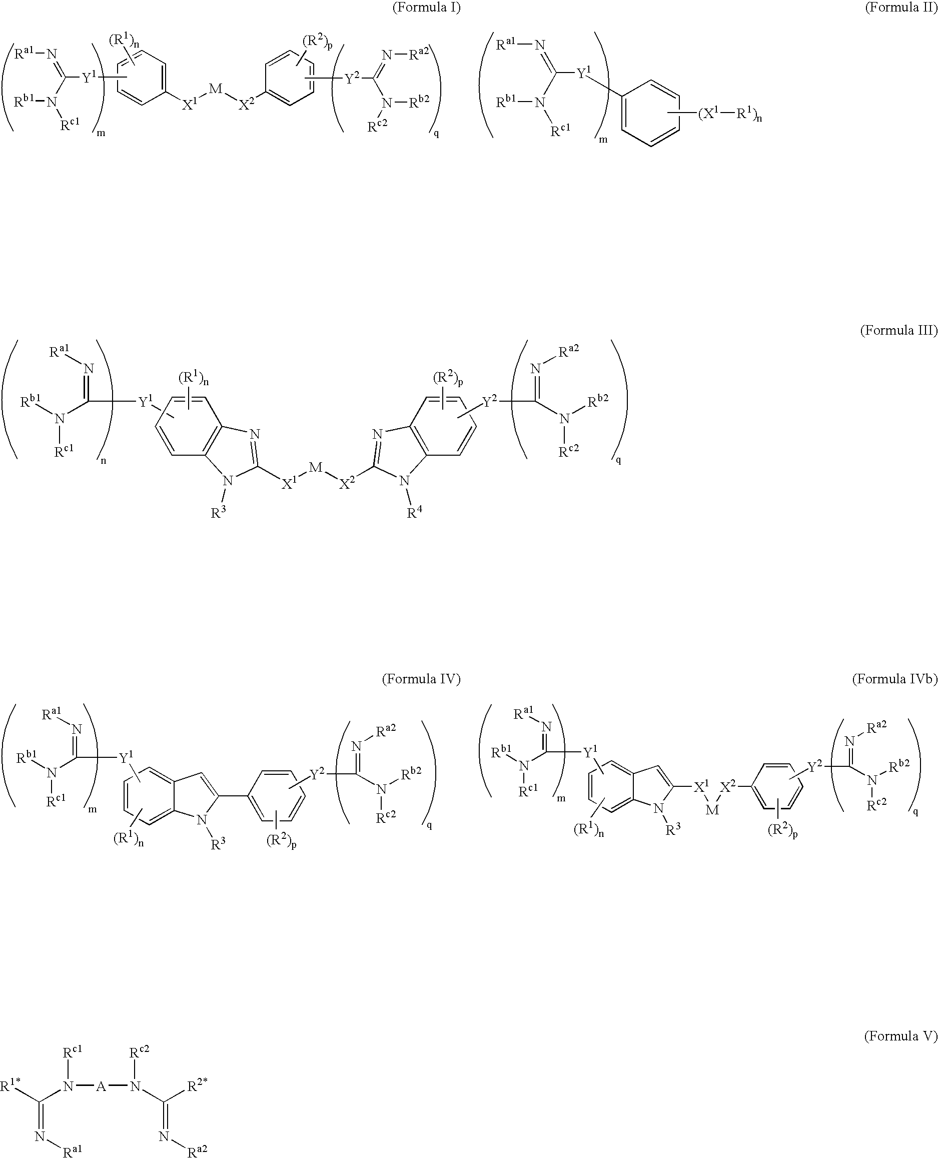

wherein R^{a1}, R^{b1}, R^{c1}, R^{a2}, R^{b2}, R^{c2}, Y¹, and Y² are as defined herein, and A is as defined above;

each of R¹ and R² is independently a hydrogen or a Z group, or two adjacent or proximate R¹ and R² groups, along with the corresponding X¹ and X² groups, if present (e.g., in Formula II), taken together with the ring (e.g., phenyl ring) to which they are bound form a fused ring structure, e.g., an aromatic or heteroaromatic (e.g., benzofuran) structure, or a cycloalkyl or heterocylic structure;

each of R³ and R⁴ is independently selected from the group consisting of hydrogen, substituted or unsubstituted straight or branched alkyl (preferably C₁-C₅), cycloalkyl (preferably C₃-C₈), carbocyclic, aryl (e.g., phenyl), heterocyclic, and heteroaryl;

each of R¹* and R²* is independently selected from the group consisting of substituted or unsubstituted straight or branched alkyl, cycloalkyl, heterocyclic, aryl (including phenyl), and heteroaryl;

each of X¹ and X² is independently a direct bond, or an oxygen, a NR′ group (where R′ is hydrogen (i.e., NH), a C₁-C₅ alkyl, C₂-C₅ alkenyl, C₂-C₅ alkynyl, or aryl group), a sulfonamide group (i.e., NHSO₂ or SO₂NH), a carbonyl, amide (i.e., NHCO or CONH), a C₁-C₅ alkylene group (e.g., —CH₂—), C₂-C₅ alkenylene group (e.g., E or Z —CH═CH—), C₂-C₅ alkynylene group, or a sulfur atom, or combinations thereof (e.g., —OCH₂—, —CH₂O—, E or Z —OCH═CH— or —CH═CHO—);

M is a divalent group such as an alkylene group, i.e., —(CH₂)ₖ— and substituted analogs thereof (including groups in which a —CH₂— moiety is substituted by an oxygen atom), where k is 1 to 12 (preferably 5 to 10, more preferably 6 to 9, most preferably 7 to 8), an alkenylene group (preferably 2 to 12 carbon atoms, more preferably 6 to 9 carbon atoms, including groups with more than one double bond), an alkynylene group (preferably 2 to 12 carbon atoms, more preferably 6 to 9 carbon atoms, including groups with more than one triple bond), an alkoxyalkylene group, an alkylaminoalkylene group, a thioalkoxyalkylene group, an arylenedialkylene group, an alkylenediarylene group, a heteroarylenedialkylene group, an arylene group, a heteroarylene group, an oligoethereal group such as an oligo(alkyleneoxide) group, or an arylene-di(oligoalkyleneoxide) group, each of which may be substituted (with, for example, a Z group as defined herein, e.g., a hydroxyalkylene group such as —(CH₂)₀₋₆(CHOH)(CH₂)₀₋₆—; or other such substituted moieties, e.g., —(CH₂)₀₋₆(CHZ)(CH₂)₀₋₆—, including —(CH₂)₀₋₆(CHCO₂alkyl)(CH₂)₀₋₆—) or unsubstituted;

Z is a substituted or unsubstituted moiety selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), NO₂, halogen (e.g., F, Cl, Br, or I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)O₃R′ (e.g., —SO₃H), (CR′R″)₀₋₃O(CR′R″)₀₋₃H (e.g., —CH₂OCH₃ and —OCH₃), (CR′R″)O₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid;

in another embodiment, Z is a substituted or unsubstituted moiety selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₁₀NR′R″ (e.g., —NH₂), (CR′R″)₀₋₁₀CN (e.g., —CN), NO₂, halogen (e.g., F, Cl, Br, or I), (CR′R″)₀₋₁₀C(halogen)₃ (e.g., —CF₃), (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′ (e.g., —SO₃H), (CR′R″)₀₋₁₀O(CR′R″)₀₋₁₀H (e.g., —CH₂OCH₃ and —OCH₃), (CR′R″)₀₋₁₀S(CR′R″)₀₋₃H (e.g., —SH and —SCH₃), (CR′R″)₁₋₁₀OH (e.g., —OH), (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₁₋₁₀CO₂R′ (e.g., —CO₂H), or (CR′R″)₀₋₁₀OR′ group, or the side chain of any naturally occurring amino acid;

wherein R′ and R″ are each independently hydrogen, a C₁-C₅ alkyl, C₂-C₅ alkenyl, C₂-C₅ alkynyl, or aryl group, or R′ and R″ taken together are a benzylidene group or a —CH₂)₂O(CH₂)₂— group;

m and q are each independently an integer selected from zero to five inclusive;

in Formula I, m and q are each independently an integer selected from zero to four inclusive, and n and p are each independently an integer selected from zero to four inclusive, such that m+n≦5 and p+q≦5, wherein either m or q is at least one; and preferably m and q are one;

in Formula II, m is an integer selected from one to six inclusive, and n is an integer selected from zero to five inclusive, such that m+n≦6;

in Formula III, m, n, p, and q are each independently an integer selected from zero to three inclusive, m+n≦4, p+q≦4, and m+q≧1 (preferably m=q=1);

in Formula IV and IVb, m and n are each independently an integer selected from zero to three inclusive, p and q are each independently an integer selected from zero to four inclusive, m+n≦4, p+q≦5, and m+q≧1 (preferably m=q=1);

and pharmaceutically acceptable salts thereof.

The chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn.

In an alternate embodiment, the invention relates to novel compounds, and novel methods of their use as described herein, which are within the scope of the Formulae disclosed herein, and which are not disclosed in the above-referenced U.S. Patents.

The groups R^{a1}, R^{b1}, R^{c1}, R^{a2}, R^{b2}, and R^{c2} in the above Formulae are preferably a hydrogen, or a substituted or unsubstituted C₁-C₈ alkyl or C₁-C₈ alkoxy group or a hydroxy group. Preferred R^{a1} and R^{a2} groups are hydrogen, hydroxyl, alkyloxy groups (especially lower alkyloxy groups, e.g. methoxy), aryloxy, acyloxy, and aroyloxy (i.e., R—(C═O)—O—, wherein R is aliphatic or aromatic).

The phrase “R^{a} and R^{b} both taken together along with the nitrogen atoms to which they are bound to form a ring structure” means that the two R^{a} and R^{b} groups are a moiety which joins the two nitrogen atoms in a heterocycle, such as the following ring structures: 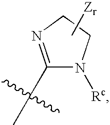
wherein r is an integer from zero to 4 inclusive, 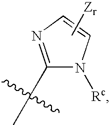
wherein r is an integer from zero to 2 inclusive, 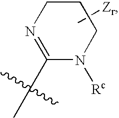
wherein r is an integer from zero to 6 inclusive,

or 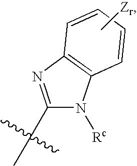
wherein r is an integer from zero to 4 inclusive.

In another embodiment of the invention, for example, in compounds of Formula II, R^{a1} and R^{b1} or R^{a2} and R^{b2} are both taken together along with the nitrogen atoms to which they are bound to form a ring structure which is a nonaromatic ring, or an alicyclic ring, or a monocyclic ring, or a non-fused ring.

In some embodiments of Formula II, e.g., R^{a1}, R^{b1}, R^{c1}, R^{a2}, R^{b2}, and R^{c2} are preferably a hydrogen, or a substituted or unsubstituted C₁-C₈ alkyl group, wherein the alkyl substituent is any member of the group Z defined above, but not an aryl (e.g., phenyl) or alkyl group. Likewise, in certain embodiments of Formula II, R¹ is a moiety selected from the Z group defined above other than an substituted aryl (e.g., phenyl) or heteroaryl group.

The groups R¹ and R² are preferably a hydrogen, a substituted or unsubstituted C₁-C₈ alkyl group, a substituted or unsubstituted C₂-C₈ alkenyl group, a halogen (particularly bromine), a substituted or unsubstituted aryl or heteroaryl group, a substituted or unsubstituted amino group, a nitro group, or a substituted or unsubstituted C₁-C₈ alkoxy group (particularly methoxy).

Each Y group may be a direct bond, or a “linking moiety” (or “linking group”) which is a group that is covalently bound to at least two other moieties and may be, for example, a single divalent atom or an oligomethylene group. A linking moiety which is a linear chain of carbon atoms may be optionally substituted or unsaturated.

Preferably a linking moiety is relatively small compared to the rest of the molecule, and more preferably less than about 250 molecular weight, and even more preferably less than about 75 molecular weight. Especially preferred linking moieties are —(CH₂)ₙ— (wherein n is 1, 2, or 3), —NR′— (where R′ is hydrogen, a C₁-C₅ alkyl, C₂-C₅ alkenyl, C₂-C₅ alkynyl, or aryl group), —S—, —O—, —NH—CH₂—, and —CH═CH— (both E and Z configurations), or combinations thereof. The linking moiety may also be (CR^{v}R^{w})ₙ, CR^{v}OR^{w}(CR^{x}R^{y})ₙ, CR^{v}SH(CR^{x}R^{y})ₙ, CR^{v}NR^{w}R^{x}(CR^{y}R^{z})ₙ, (CR^{v}R^{w})ₙO(CR^{x}R^{y})ₙ, wherein each n is independently either 0, 1, 2, or 3, and R^{v}, R^{w}, R^{x}, R^{y}, and R^{z} are each independently hydrogen, a substituted or unsubstituted C₁-C₅ branched or straight chain alkyl or alkoxy, C₂-C₅ branched or straight chain alkenyl, aryloxycarbonyl, arylaminocarbonyl, arylalkyl, acyl, aryl, or C₃-C₈ ring group.

“Inhibition” of amyloid deposition includes preventing or stopping of amyloid formation, e.g., fibrillogenesis, inhibiting or slowing down of further amyloid deposition in a subject with amyloidosis, e.g., already having amyloid deposits, and reducing or reversing amyloid fibrillogenesis or deposits in a subject with ongoing amyloidosis. Inhibition of amyloid deposition is determined relative to an untreated subject, or relative to the treated subject prior to treatment, or, e.g., determined by clinically measurable improvement in pancreatic function in a diabetic patient, or in the case of a patient with brain amyloidosis, e.g., an Alzheimer's or cerebral amyloid angiopathy patient, stabilization of cognitive function or prevention of a further decrease in cognitive function (i.e., preventing, slowing, or stopping disease progression).

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl, etc.), alkyl substituted cycloalkyl groups, and cycloalkyl substituted alkyl groups. Unless otherwise specified, the term alkyl further includes alkyl groups, which can further include oxygen, nitrogen, sulfur or phosphorous atoms replacing one or more carbons of the hydrocarbon backbone.

In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms. An “alkylene” group is a divalent moiety derived from the corresponding alkyl group.

Moreover, unless otherwise specified the term alkyl includes both “unsubstituted alkyls” and “substituted alkyls,” the latter of which refers to alkyl moieties having substituents replacing one or more hydrogens on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls may be further substituted, e.g., with the substituents described above.

An “arylalkyl” moiety is an alkyl group substituted with an aryl (e.g., phenylmethyl (i.e., benzyl)). An “alkylaryl” moiety is an aryl group substituted with an alkyl group (e.g., p-methylphenyl (i.e., p-tolyl)). The term “n-alkyl” means a straight chain (i.e., unbranched) unsubstituted alkyl group.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclobutenyl, cyclopentenyl, cyclohexenyl, cycloheptenyl, cyclooctenyl, etc.), alkyl or alkenyl substituted cycloalkenyl groups, and cycloalkyl or cycloalkenyl substituted alkenyl groups. The term alkenyl may further include alkenyl groups which include oxygen, nitrogen, sulfur or phosphorous atoms replacing one or more carbons of the hydrocarbon backbone.

In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms. An “alkenylene” group is a divalent moiety derived from the corresponding alkenyl group.

Moreover, unless otherwise specified the term alkenyl includes both “unsubstituted alkenyls” and “substituted alkenyls,” the latter of which refers to alkenyl moieties having substituents replacing one or more hydrogens on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate (and lower alkyl esters thereof), alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. Unless specified otherwise, the term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms. An “alkynylene” group is a divalent moiety derived from the corresponding alkynyl group.

Moreover, unless otherwise specified the term alkynyl includes both “unsubstituted alkynyls” and “substituted alkynyls,” the latter of which refers to alkynyl moieties having substituents replacing one or more hydrogens on one or more carbons of the hydrocarbon backbone.

Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “acyl” refers to a carbonyl group that is attached through its carbon atom to a hydrogen (i.e., a formyl), an aliphatic group (e.g., acetyl), an aromatic group (e.g., benzoyl), and the like. The term “substituted acyl” includes acyl groups where one or more of the hydrogen atoms on one or more carbon atoms are replaced by, for example, an alkyl group, alkynyl group, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an amino moiety is bonded to an acyl group. For example, the acylamino group includes alkylcarbonylamino, arylcarbonylamino, carbamoyl and ureido groups.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone.

The terms “alkoxy” or “alkyloxy” include substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups. Examples of substituted alkoxy groups include halogenated alkoxy groups.

The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc., as well as perhalogenated alkyloxy groups.

The term “amine” or “amino” includes compounds or moieties in which a nitrogen atom is covalently bonded to at least one carbon or heteroatom.

The term “alkylamino” includes groups wherein the nitrogen is bound to at least one alkyl group. The term “dialkylamino” includes groups wherein the nitrogen atom is bound to at least two alkyl groups.

The term “arylamino” and “diarylamino” include groups wherein the nitrogen is bound to at least one or two aryl groups, respectively.

The term “alkylarylamino” refers to an amino group which is bound to at least one alkyl group and at least one aryl group.

The term “alkaminoalkyl” refers to an alkyl, alkenyl, or alkynyl group substituted with an alkylamino group.

The term “amide” or “aminocarbonyl” includes compounds or moieties which contain a nitrogen atom which is bound to the carbon of a carbonyl or a thiocarbonyl group.

The term “carbonyl” or “carboxy” includes compounds and moieties which contain a carbon connected with a double bond to an oxygen atom. Examples of moieties which contain a carbonyl include aldehydes, ketones, carboxylic acids, amides, esters, anhydrides, etc.

The term “ether” or “ethereal” includes compounds or moieties which contain an oxygen bonded to two carbon atoms. For example, an ether or ethereal group includes “alkoxyalkyl” which refers to an alkyl, alkenyl, or alkynyl group substituted with an alkoxy group.

The term “hydroxy” or “hydroxyl” includes the groups —OH or —O⁻ (with an appropriate counter ion).

The term “halogen” includes fluorine, bromine, chlorine, iodine, etc. The term “perhalogenated” generally refers to a moiety wherein all hydrogens are replaced by halogen atoms.

Arylenedialkylene or arylenedialkyl groups include those groups which have an arylene group to which are bound two other alkylene groups, which may be the same or different, and which two alkylene groups are in turn bound to other moieties. Examples of arylenedialkylene or arylenedialkyl groups include the following: 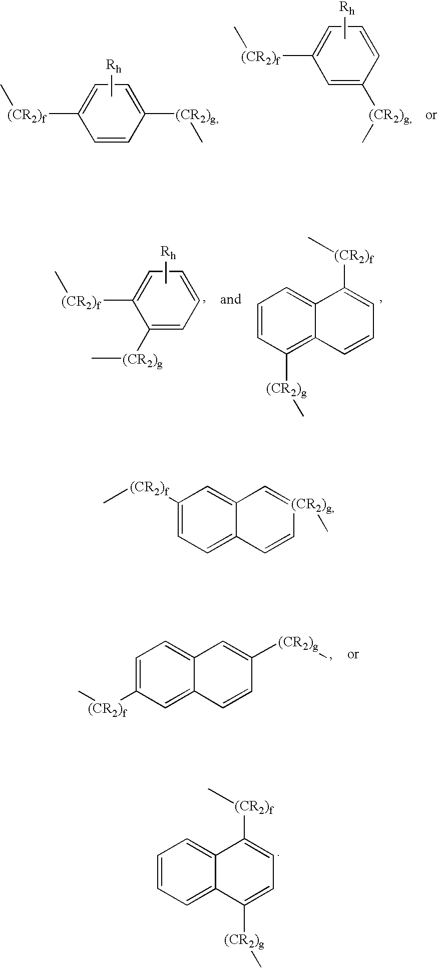
wherein each R group is independently a hydrogen (preferred) or is selected from the group Z defined above, and 1≦f≦8, 1≦g≦8, 0≦h≦4.

Alkylenediarylene groups include groups which have an alkylene (or cycloalkylene) group to which are bound two other arylene groups, which may be the same or different, and which two alkylene groups are in turn bound to other moieties. Examples of alkylenediarylene groups include the following: 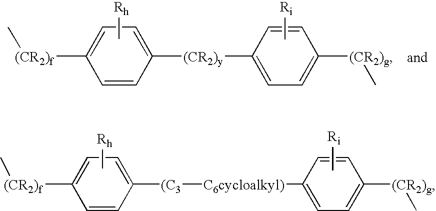
wherein each R group is independently a hydrogen (preferred) or is selected from the group Z defined above, 1≦y≦10 (preferably 1≦y≦4), 1≦f≦8, 1≦g≦8, 0≦h≦4, and 0≦i≦4.

Heteroarylenedialkylene or heteroarylenedialkyl groups include those groups which have a heteroarylene group to which are bound two other alkylene groups, which may be the same or different, and which two alkylene groups are in turn bound to other moieties. Examples of heteroarylenedialkylene or heteroarylenedialkyl groups include the following: 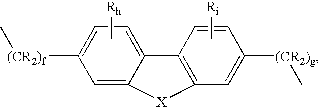
wherein 0≦h≦3, and 0≦i≦3, and X=NR′ (wherein R′ is hydrogen, a C₁-C₅ alkyl, C₂-C₅ alkenyl, C₂-C₅ alkynyl, or aryl group), O, or S, 1≦f≦8, 1≦g≦8. 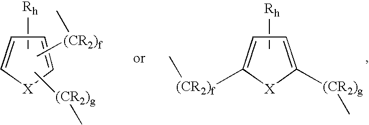
wherein 0≦h≦2, and X=NR′ (wherein R′ is hydrogen, a C₁-C₅ alkyl, C₂-C₅ alkenyl, C₂-C₅ alkynyl, or aryl group), O, or S, 1≦f≦8, 1≦g≦8. 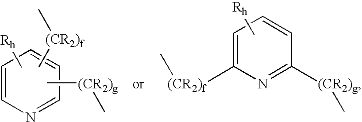
wherein 0≦h≦3, 1≦f≦8, 1≦g≦8, or 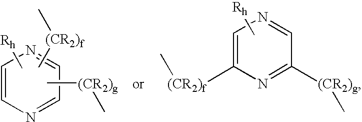
wherein 0≦h≦2,
wherein each R group is independently a hydrogen (preferred) or is selected from the group Z defined above, 1≦f≦8, 1≦g≦8, and h and i are as indicated.

An arylene group is an aromatic group which is capable of being connected covalently to other substituents through at least two positions, including the following examples: 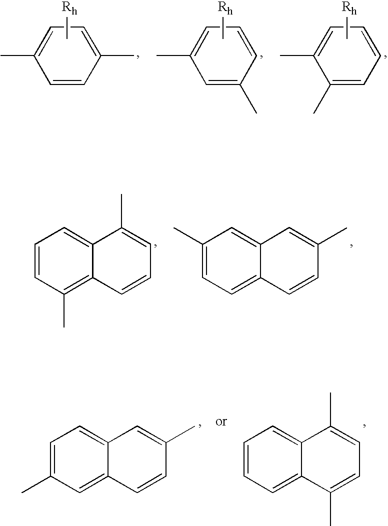
wherein each R group is independently a hydrogen (preferred) or is selected from the group Z defined above, and 0≦h≦4; for example:

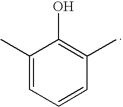

A heteroarylene group is a heteroaromatic group which is capable of being connected covalently to other substituents through at least two positions, including the following examples: 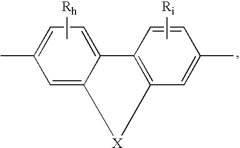
wherein 0≦h≦3, and 0≦i≦3, and X=NR′ (wherein R′ is hydrogen, a C₁-C₅ alkyl, C₂-C₅ alkenyl, C₂-C₅ alkynyl, or aryl group), O, or S, 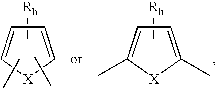
wherein 0≦h≦2, and X=NR′ (wherein R′ is hydrogen, a C₁-C₅ alkyl, C₂-C₅ alkenyl, C₂-C₅ alkynyl, or aryl group), O, or S, 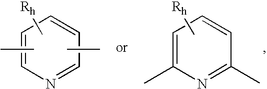
wherein 0≦h≦3, or 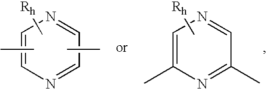
wherein 0≦h≦2,
wherein each R group is independently a hydrogen (preferred) or is selected from the group Z defined above, and h and i are as indicated; for example, the following groups:

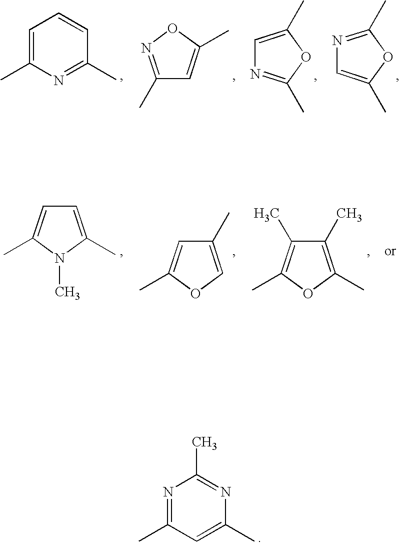

Likewise, the invention relates to the following heteroarylene groups 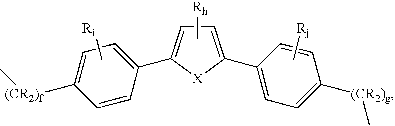
wherein X=NR′ (wherein R′ is hydrogen, a C₁-C₅ alkyl, C₂-C₅ alkenyl, C₂-C₅ alkynyl, or aryl group), O, or S, 0≦f≦8, 0≦g≦8; and each R group is independently a hydrogen (preferred) or is selected from the group Z defined above.

In general, the term “aryl” includes groups, including 5- and 6-membered single-ring aromatic groups that may include from zero to four heteroatoms, for example, groups derived from benzene, pyrrole, furan, thiophene, thiazole, isothiaozole, imidazole, triazole, tetrazole, pyrazole, oxazole, isooxazole, pyridine, pyrazine, pyridazine, and pyrimidine, and the like.

Furthermore, the term “aryl” includes multicyclic aryl groups, e.g., groups derived from tricyclic, bicyclic, e.g., naphthalene, benzoxazole, benzodioxazole, benzothiazole, benzoimidazole, benzothiophene, methylenedioxyphenyl, quinoline, isoquinoline, napthyridine, indole, benzofuran, purine, benzofuran, deazapurine, or indolizine.

Those aryl groups having heteroatoms in the ring structure may also be referred to as “aryl heterocycles,” “heterocycles,” “heteroaryls” or “heteroaromatics”.

An aromatic ring can be substituted at one or more ring positions with such substituents as described above, as for example, halogen, hydroxyl, alkyl (e.g. tolyl), alkoxy, alkylcarbonyloxy, arylcarbonyloxy, alkoxycarbonyloxy, aryloxycarbonyloxy, carboxylate, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Aryl groups can also be fused or bridged with alicyclic or heterocyclic rings which are not aromatic so as to form a polycycle (e.g., tetralin).

The term “heterocyclic” or “heterocycle” includes heteroaryls as well as any ring formed which incorporate a heteroatom or an atom which is not carbon. The ring may be saturated or unsaturated and may contain one or more double bonds. Examples of preferred heterocyclic groups include pyridyl, furanyl, thiophenyl, morpholinyl, and indolyl groups. The term “heteroatom” includes atoms of any element other than carbon or hydrogen. Preferred heteroatoms are nitrogen, oxygen, sulfur and phosphorus.

An “arylene” group is a divalent moiety derived from an aryl group.

An oligoethereal group, such as an oligo(alkyleneoxide) group, includes polyethyleneglycol (PEG) and short chain analogs thereof including —[(CR₂)ₛO]ₜ(CR₂)ₛ—, wherein 1≦t≦6 and 1≦s≦6, and each R group is independently a hydrogen (preferred) or is selected from the group Z defined above.

An arylene-di(oligoalkyleneoxide) group is an aryl group which has two oligoalkyleneoxide groups bound to it which in turn are bound to other moieties, and include the following examples: 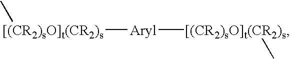
wherein “Aryl” is an arylene moiety, 1≦t≦6, 0≦s≦6, and each R group is independently a hydrogen (preferred) or is selected from the group Z defined above. Preferred arylene-di(oligoalkyleneoxide) groups include: 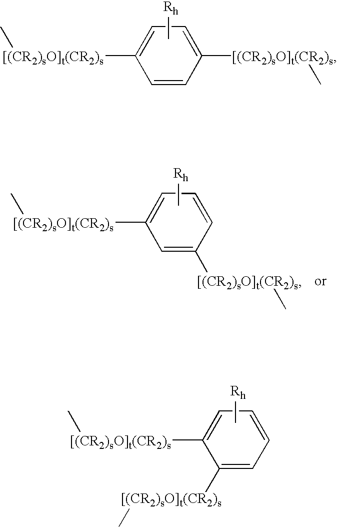
wherein 1≦t≦6, 0≦s≦6, 0≦h≦4, and each R group is independently a hydrogen (preferred) or is selected from the group Z defined above.

The term “substituted” means that the moiety has substituents placed on the moiety other than hydrogen which allow the molecule to perform its intended function. Examples of substituents include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), NO₂, halogen (e.g., F, Cl, Br, or I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group. Preferably, substitutions enhance the ability of the compounds of the invention to perform its intended function, e.g., inhibit formation of amyloid deposits.

In compounds of the invention, it is preferred that m=1 and that n=0, 1, or 2. In compounds of Formula I, preferably p=0, 1, or 2, and q=1. It is especially preferred that molecules according to Formula I are symmetric, thus R^{a1}=R^{a2}, R^{b1}=R^{b2}, R^{c1}=R^{c2}, m=q, n=p, and Y¹=Y². Likewise, it is preferred that R¹=R², and X¹=X² in molecules of Formula I.

One group of preferred compounds of the invention are those of Formula Ia: 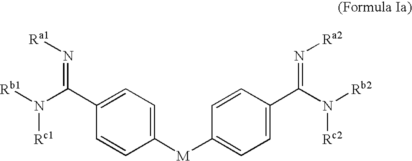
wherein M is 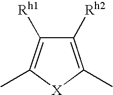
wherein, in a preferred aspect, R^{a1} and R^{b1} together, or R^{a2} and R^{b2} together, represent a C₂ to C₃ alkylene; R^{c1} and R^{c2} are H; R^{h1} is H; and R^{h2} is OCH₃ or O(C₆H₄)R, wherein R is H or lower-alkyl, and X is O, NR′ (wherein R′ is hydrogen, a C₁-C₅ alkyl, C₂-C₅ alkenyl, C₂-C₅ alkynyl, or aryl group), or S.

In another group of preferred compounds of Formula Ia, R^{a1} and R^{b1} together, or R^{a2} and R^{b2} together, represent a C₂ linear, saturated alkylene; R^{c1} and R^{c2} are -(lower alkyl)-OH; and R^{h1} and R^{h2} are each H. The “lower alkyl” group of R^{c1} and R^{c2} are preferably ethylene.

In yet another group of preferred compounds of Formula Ia, R^{a1} and R^{b1} together, or R^{a2} and R^{b2} together, represent a C₄ alkylene; R^{c1} and R^{c2} are H (preferred), lower alkyl, cycloalkyl, aryl, hydroxyalkyl, aminoalkyl or alkylaminoalkyl; R^{h1} and R^{h2} are independently selected from the group consisting of H (preferred), lower alkyl, halogen, alkoxy, aryloxy, or arylalkoxy.

In still yet another group of preferred compounds of Formula Ia, R^{a1}, R^{a2}, R^{b1} and R^{b2} are H; R^{c1} and R^{c2} are isopropyl or —(CH₂)₃N(CH₃)₂; and R^{h1} and R^{h2} are H.

In a further group of preferred compounds of Formula Ia, R^{a1} and R^{b1} together, or R^{a2} and R^{b2} together, represent a phenylene group which is optionally substituted with up to three —CONHR^{d}NR^{c}R^{f} groups where R^{d} is lower alkyl and R^{e} and R^{f} are each independently selected from the group consisting of H or lower alkyl; and R^{c1}, R^{c2}, R^{h1}, and R^{h2} are H.

An especially preferred compound of Formula Ia has R^{h1}, R^{h2}, R^{b1}, R^{c1}, R^{b2}, and R^{c2} being H, and R^{a1} and R^{a2} groups being hydroxy or methoxy.

Another group of preferred compounds are those of Formula Ib: 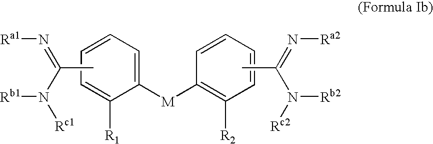
wherein M is 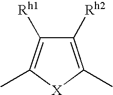
wherein X is O, NR′ (wherein R′ is hydrogen, a C₁-C₅ alkyl, C₂-C₅ alkenyl, C₂-C₅ alkynyl, or aryl group), or S; R^{h1} and R^{h2} are each independently selected from the group consisting of H, loweralkyl, aryl, alkylaryl, aminoalkyl, aminoaryl, halogen, alkoxy, aryloxy, or oxyarylalkyl; R¹ and R² are each independently selected from the group consisting of H, loweralkyl, alkoxy, alkylaryl, aryl, aryloxy, aminoalkyl, aminoaryl, or halogen; and each R^{a1}, R^{a2}, R^{b1}, and R^{b2} group is independently selected from the group consisting of H, loweralkyl, alkoxyalkyl, hydroxyalkyl, aminoalkyl, alkylaminoalkyl, cycloalkyl, aryl, hydroxy, or alkylaryl; or R^{a1} and R^{b1} together, or R^{a2} and R^{b2} together, represent C₂-C₁₀ alkyl, hydroxyalkyl, or alkylene; and each R^{c1} and R^{c2} group is independently H, hydroxy, loweralkyl, alkoxyalkyl, hydroxyalkyl, aminoalkyl, alkylamino, alkylaminoalkyl, cycloalkyl, hydroxycycloalkyl, alkoxycycloalkyl, aryl, or alkylaryl.

Another group of preferred compounds are those of Formula Ic: 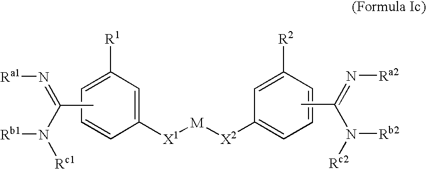
wherein M is 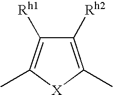
wherein X is S, O, or NR′ (wherein R′ is hydrogen, a C₁-C₅ alkyl, C₂-C₅ alkenyl, C₂-C₅ alkynyl, or aryl group); R^{b1}, R^{b2}, R^{c1}, and R^{c2} are each independently selected from the group consisting of H, loweralkyl, alkoxy, alkoxyalkyl, cycloalkyl, aryl, hydroxyalkyl, aminoalkyl or alkylaminoalkyl; R¹ and R² are H, lower alkyl, alkoxy, alkoxyalkyl, hydroxyalkyl, cycloalkyl, aryl, aminoalkyl, alkylaminoalkyl or halogen; R^{a1} and R^{a2} are —OY, or R^{a1} and R^{b1} together, or R^{a2} and R^{b2} together represent 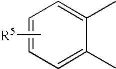
wherein R⁵ is 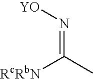
Y is H or lower alkyl; each of X¹ and X² are —(CH₂)ₙ—, where n is an integer from 0 to 2; and R^{h1} and R^{h2} are each independently selected from the group consisting of H, lower alkyl, halogen, alkoxy, aryloxy, or oxyarylalkyl.

Yet another group of preferred compounds are those of Formula Ic, wherein M is —(CH₂)ₙ— where n is an integer from 2 to 16 (or 2 to 12, or 2 to 10); each of X¹ and X² is O, NH, or S; R^{a1}, R^{a2}, R^{b1}, and R^{b2} are H; or R^{a1} and R^{b1} together, or R^{a2} and R^{b2} together represent —(CH₂)ₘ—, wherein m is 2, 3, or 4; each of R¹ and R² are H, OCH₃, NO₂ or NH₂; R^{c1} and R^{c2} are H, CH₃ or CH₂CH₃. In another embodiment, when X¹ is O or S, both R¹ and R^{c1} cannot be H; and when X² is O or S, both R² and R^{c2} cannot be H.

Another group of preferred compounds are those of Formula Id: 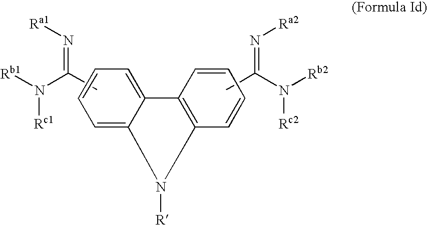
wherein each R^{a1}, R^{a2}, R^{b1}, and R^{b2} are independently selected from the group consisting of H, loweralkyl, alkoxyalkyl, hydroxyalkyl, aminoalkyl, alkylaminoalkyl, cycloalkyl, aryl, or alkylaryl; or two R^{a1} and R^{b1} together, or R^{a2} and R^{v2} together represent C₂-C₁₀ alkylene; R^{c1} and R^{c2} are independently H, hydroxy, loweralkyl, alkoxyalkyl, hydroxyalkyl, aminoalkyl, alkylaminoalkyl, cycloalkyl, aryl, or alkylaryl; and R′ is H, loweralkyl, alkoxyalkyl, hydroxyalkyl, aminoalkyl, alkylaminoalkyl, cycloalkyl, aryl, or alkylaryl.

Another group of preferred compounds are those of Formula Ie: 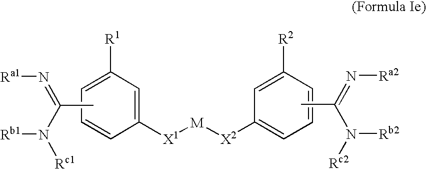
wherein M is an alkylene group (e.g., C₂ to C₁₆), and X¹ and X² are oxygen.

In another group of preferred compounds of Formula Ie, R^{a1} and R^{b1} together, or R^{a2} and R^{b2} together, represent a C₂ linear, saturated alkylene; R^{c1} and R^{c2} are H.

Another group of preferred compounds of the invention are those of Formula Ia: 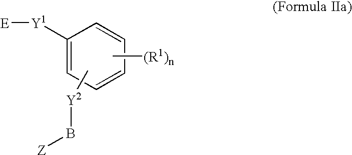
wherein E is 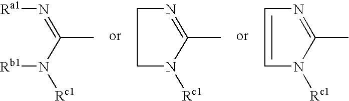
wherein Y¹, Y², Z, and R¹ are as defined above; n is 0-4; Y² is preferably O, NH, S, a substituted or unsubstituted methylene group, or a direct bond; Z may be a hydrogen atom, or Z is preferably alkyl, aryl, alkoxy, aryloxy, hydroxy, a substituted or unsubstituted amino, nitro, sulfo, or halogen group; R^{a1}, R^{b1}, and R^{c1} are independently hydrogen, lower alkyl, aromatic, hydroxyl, or alkoxy; and B is a direct bond or a substituted or unsubstituted alkylene group containing from 1 to 16 carbon atoms, or a biphenylene group, or a combination biphenylene-alkylene group, the group —[(CH₂)ₙO]ₘ(CH₂)ₙ— where m is 1 to 6 and n is 2 to 6, or a heterocyclic group.

Compounds of Formula IIb are also within the invention: 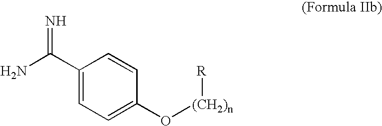
wherein n=2, 3, 4, 5, 6, 7, 8, 9, or 10; and R=hydrogen, hydroxy, halogen, phenyl, biphenyl, naphthyl, alkoxy, carboxy, alkoxycarbonyl, aryloxycarbonyl, or aryloxy.

Another group of preferred compounds are of Formula IIa: 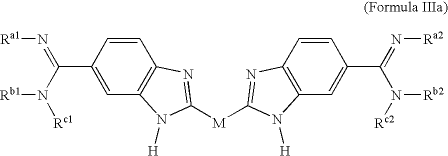
wherein M is 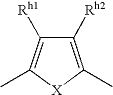
wherein X is S, O, or NR′ (wherein R′ is hydrogen, a C₁-C₅ alkyl, C₂-C₅ alkenyl, C₂-C₅ alkynyl, or aryl group); R^{a1}, R^{a2}, R^{b1}, and R^{b2} are each independently selected from the group consisting of H, lower alkyl, alkoxyalkyl, cycloalkyl, aryl, alkylaryl, hydroxyalkyl, aminoalkyl, or alkylaminoalkyl; or R^{a1} and R^{b1} together, or R^{a2} and R^{b2} together represent a C₂ to C₁₀ alkyl, hydroxyalkyl, or alkylene; or R^{a1} and R^{b1} together, or R^{a2} and R^{b2} together are: 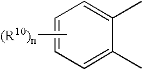
wherein n is a number from 1 to 3, and R¹⁰ is H or —CONHR¹¹NR¹⁵R¹⁶, wherein R¹¹ is lower alkyl and R¹⁵ and R¹⁶ are each independently selected from the group consisting of H and lower alkyl; and R^{c1} and R^{c2} are H, hydroxy, lower alkyl, cycloalkyl, aryl, alkylaryl, alkoxyalkyl, hydroxycycloalkyl, alkoxycycloalkoxy, hydroxyalkyl, aminoalkyl or alkylaminoalkyl; and R^{h1} and R^{h2} are each independently selected from the group consisting of H, lower alkyl, halogen, aryl, arylalkyl, aminoalkyl, aminoaryl, alkoxy, aryloxy, or oxyarylalkyl.

Yet another group of preferred compounds are of Formula IIIb: 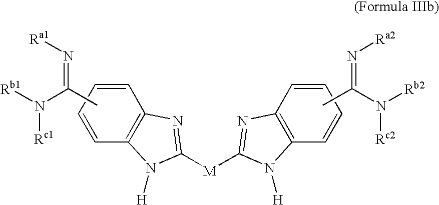
wherein each pair of R^{a1} with R^{b1} and R^{a2} with R^{b2} together represent —(CH₂)ₘ— wherein m is from two to four; R^{c1} and R^{c2} are independently H or loweralkyl; and M, which may be substituted with a lower alkyl group, is selected from the group consisting of —CH═CH—CH₂—CH₂—, —CH₂—CH═CH—CH₂—, and —CH═CH—CH═CH—.

Another group of preferred compounds are those of Formula IIIc: 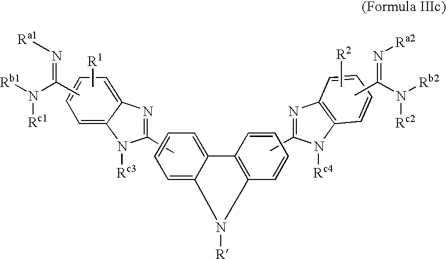
wherein R¹ and R² are independently H or —CONHR⁵NR⁶R⁷, wherein R⁵ is lower alkyl, R⁶ and R⁷ are each independently selected from the group consisting of H and lower alkyl; R^{a1}, R^{a2}, R^{b1}, and R^{b2} are independently selected from the group consisting of H, lower alkyl, alkoxyalkyl, hydroxyalkyl, aminoalkyl, alkylaminoalkyl, cycloalkyl, aryl, or alkylaryl, or R^{a1} and R^{b1} together, or R^{a2} and R^{b2} together represent C₂-C₁₀ alkylene; R^{c1} and R^{c2} are independently H, hydroxy, lower alkyl, alkoxyalkyl, hydroxyalkyl, aminoalkyl, alkylaminoalkyl, cycloalkyl, aryl, or alkylaryl; R^{c3} and R^{c4} are independently H, hydroxy, loweralkyl, alkoxyalkyl, hydroxyalkyl, aminoalkyl, alkylaminoalkyl, cycloalkyl, aryl, or alkylaryl; and R′ is H, loweralkyl, alkoxyalkyl, hydroxyalkyl, aminoalkyl, alkylaminoalkyl, cycloalkyl, aryl, alkylaryl, or halogen.

In another embodiment, the present invention relates to pharmaceutical compositions comprising compounds according to any of the Formulae herein for the treatment of an amyloid-related disease, as well as methods of manufacturing such pharmaceutical compositions.

The compounds of the invention can be formulated to ensure proper distribution in vivo. For example, the blood-brain barrier (BBB) excludes many highly hydrophilic compounds. To ensure that the more hydrophilic therapeutic compounds of the invention cross the BBB, they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties”), thus providing targeted drug delivery (see, e.g., V. V. Ranade (1989) J. Clin. Pharmacol. 29:685).

Exemplary targeting moieties include folate or biotin (see, e.g., U.S. Pat. No. 5,416,016 to Low et al.); mannosides (Umezawa et al. (1988) Biochem. Biophys. Res. Commun. 153:1038); antibodies (P. G. Bloeman et al. (1995) FEBS Lett. 357:140; M. Owais et al. (1995) Antimicrob. Agents Chemother. 39:180); surfactant protein A receptor (Briscoe et al. (1995) Am. J. Physiol. 1233:134); gp120 (Schreier et al. (1994) J. Biol. Chem. 269:9090); see also K. Keinanen; M. L. Laukkanen (1994) FEBS Lett. 346:123; J. J. Killion; I. J. Fidler (1994) Immunomethods 4:273. In a preferred embodiment, the therapeutic compounds of the invention are formulated in liposomes; in a more preferred embodiment, the liposomes include a targeting moiety.

To ensure that compounds of the invention cross the BBB, they may be coupled to a BBB transport vector (for review of BBB transport vectors and mechanisms, see Bickel, et al., Adv. Drug Delivery Reviews, vol. 46, pp. 247-279, 2001). Exemplary transport vectors include cationized albumin or the OX26 monoclonal antibody to the transferrin receptor; these proteins undergo absorptive-mediated and receptor-mediated transcytosis through the BBB, respectively.

Examples of other BBB transport vectors that target receptor-mediated transport systems into the brain include factors such as insulin, insulin-like growth factors (IGF-I, IGF-II), angiotensin II, atrial and brain natriuretic peptide (ANP, BNP), interleukin I (IL-1) and transferrin. Monoclonal antibodies to the receptors which bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized immunoglobulins. Small basic oligopeptides such as the dynorphin analogue E-2078 and the ACTH analogue ebiratide can also cross the brain via absorptive-mediated transcytosis and are potential transport vectors.

Other BBB transport vectors target systems for transporting nutrients into the brain. Examples of such BBB transport vectors include hexose moieties, e.g. glucose, monocarboxylic acids, e.g. lactic acid, neutral amino acids, e.g. phenylalanine, amines, e.g. choline, basic amino acids, e.g. arginine, nucleosides, e.g. adenosine, purine bases, e.g. adenine, and thyroid hormone, e.g. triiodothyridine. Antibodies to the extracellular domain of nutrient transporters can also be used as transport vectors. Other possible vectors include angiotensin II and ANP, which may be involved in regulating BBB permeability.

In some cases, the bond linking the therapeutic compound to the transport vector may be cleaved following transport into the brain in order to liberate the biologically active compound. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector.

To administer the therapeutic compound by other than parenteral administration, it may be necessary to coat the compound with, or co-administer the compound with, a material to prevent its inactivation. For example, the therapeutic compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., (1984) J. Neuroimmunol. 7:27).

The therapeutic compound may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi.

The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of amyloid deposition in subjects.

The present invention therefore includes pharmaceutical formulations comprising the compounds of the Formulae described herein, including pharmaceutically acceptable salts thereof, in pharmaceutically acceptable carriers for aerosol, oral and parenteral administration. Also, the present invention includes such compounds, or salts thereof, which have been lyophilized and which may be reconstituted to form pharmaceutically acceptable formulations for administration, as by intravenous, intramuscular, or subcutaneous injection. Administration may also be intradermal or transdermal.

In accordance with the present invention, a compound of the Formulae described herein, and pharmaceutically acceptable salts thereof, may be administered orally or through inhalation as a solid, or may be administered intramuscularly or intravenously as a solution, suspension or emulsion. Alternatively, the compounds or salts may also be administered by inhalation, intravenously or intramuscularly as a liposomal suspension.

Pharmaceutical formulations are also provided which are suitable for administration as an aerosol, by inhalation. These formulations comprise a solution or suspension of the desired compound of any Formula herein, or a salt thereof, or a plurality of solid particles of the compound or salt. The desired formulation may be placed in a small chamber and nebulized. Nebulization may be accomplished by compressed air or by ultrasonic energy to form a plurality of liquid droplets or solid particles comprising the compounds or salts. The liquid droplets or solid particles should have a particle size in the range of about 0.5 to about 5 microns. The solid particles can be obtained by processing the solid compound of any Formula described herein, or a salt thereof, in any appropriate manner known in the art, such as by micronization. Most preferably, the size of the solid particles or droplets will be from about 1 to about 2 microns. In this respect, commercial nebulizers are available to achieve this purpose.

Preferably, when the pharmaceutical formulation suitable for administration as an aerosol is in the form of a liquid, the formulation will comprise a water-soluble compound of any Formula described herein, or a salt thereof, in a carrier which comprises water. A surfactant may be present which lowers the surface tension of the formulation sufficiently to result in the formation of droplets within the desired size range when subjected to nebulization.

Active compounds are administered at a therapeutically effective dosage sufficient to inhibit amyloid deposition in a subject. A “therapeutically effective” dosage preferably inhibits amyloid deposition by at least about 20%, more preferably by at least about 40%, even more preferably by at least about 60%, and still more preferably by at least about 80% relative to untreated subjects. In the case of an Alzheimer's patient, a “therapeutically effective” dosage stabilizes cognitive function or prevents a further decrease in cognitive function (i.e., preventing, slowing, or stopping disease progression).

The ability of a compound to inhibit amyloid deposition can be evaluated in an animal model system that may be predictive of efficacy in inhibiting amyloid deposition in human diseases, such as a transgenic mouse expressing human APP or other relevant animal models where Aβ deposition is seen. Likewise, the ability of a compound to prevent or reduce cognitive impairment in a model system may be indicative of efficacy in humans. Alternatively, the ability of a compound can be evaluated by examining the ability of the compound to inhibit amyloid fibril formation in vitro, e.g., using a fibrillogenesis assay such as that described herein, including a ThT, CD, or EM assay. Also the binding of a compound to amyloid fibrils may be measured using a MS assay as described herein.

The present invention is also related to prodrugs of the compounds of the Formulae disclosed herein. Prodrugs are compounds which are converted in vivo to active forms (see, e.g., R. B. Silverman, 1992, “The Organic Chemistry of Drug Design and Drug Action,” Academic Press, Chp. 8). Prodrugs can be used to alter the biodistribution (e.g., to allow compounds which would not typically enter the reactive site of the protease) or the pharmacokinetics for a particular compound. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (e.g., acyloxymethyl esters) which are cleaved to reveal an intermediate compound which subsequently decomposes to yield the active compound. The prodrug moieties may be metabolized in vivo by esterases or by other mechanisms to carboxylic acids.

Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties, (e.g., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, dilower alkyl amides, and hydroxy amides.

It will be noted that the structures of some of the compounds of this invention include stereogenic carbon atoms. It is to be understood accordingly that the isomers arising from such asymmetry (e.g., all enantiomers and diastereomers) are included within the scope of this invention unless indicated otherwise. That is, unless otherwise stipulated, any chiral carbon center may be of either (R)- or (S)-stereochemistry. Such isomers can be obtained in substantially pure form by classical separation techniques and by stereochemically controlled synthesis. Furthermore, alkenes can include either the E- or Z-geometry, where appropriate.

Certain embodiments of the present compounds can contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed.

Representative salts include the hydrohalide (including hydrobromide and hydrochloride), sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, 2-hydroxyethylsulfonate, and laurylsulphonate salts and the like. (See, e.g., Berge et al. (1977) “Pharmaceutical Salts”, J. Pharm. Sci. 66:1-19).

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention.

These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like.

Those skilled in the art will recognize, or be able to ascertain using no more than routine experimentation, many equivalents to the specific embodiments and methods described herein. Such equivalents are intended to be encompassed by the scope of the following claims. All patents, patent applications, and literature references cited herein are hereby expressly incorporated by reference in their entirety. This invention is further illustrated by the following examples which should not be construed as limiting.

### EXAMPLES

The synthesis of amidine compounds of the invention is described in U.S. Pat. Nos. 5,428,051, 4,963,589, 5,202,320, 5,935,982, 5,521,189, 5,686,456, 5,627,184, 5,622,955, 5,606,058, 5,668,167, 5,667,975, 6,025,398, 6,214,883, 5,817,687, 5,792,782, 5,939,440, 6,017,941, 5,972,969, 6,046,226, 6,294,565 (B1), 6,156,779, 6,326,395, 6,008,247, 6,127,554, 6,172,104, 4,940,723, 5,206,236, 5,843,980, 4,933,347, 5,668,166, 5,817,686, 5,723,495, 4,619,942, 5,792,782, 5,639,755, 5,643,935, 5,602,172, 5,594,138, and 5,578,631. Many of the compounds may also be purchased from Sigma-Aldrich Co. (Milwaukee, USA). The compounds may also be synthesized according to art-recognized techniques.

Test compounds were purchased from commercial sources or synthesized and screened by thioflavin T fluorescent assay (“ThT assay”). Alternatively, one could screen test compounds by circular dichroism (“CD”), electron microscopy (“EM”), or mass spectroscopy (“MS”) assays. The MS assay gives data on the ability of compounds to bind to an amyloid protein, whereas the ThT, EM, and CD assays give data on inhibition of fibrillogenesis.

The thioflavin T fluorescent assay for fibrillogenesis is based on the principle that the fluorescent dye, thioflavin T, binds specifically to fibrillar, but not to unaggregate Aβ peptide (LeVine III, H., 1993, Protein Science 2:404-410). Upon binding, thioflavin T develops a characteristic fluorescence (Naiki, H., et al., 1996, Lab. Invest. 74: 374-383) which can be easily detected. The dye is believed to interact with the stacked cross-0 pleated sheets, the common structural motif of all amyloid (LeVine III, H., 1995, Amyloid: Int. J. Exp. Clin Invest. 2:1.6). Thioflavin T is widely used to assay the effect of compounds on fibrillogenesis of Aβ peptide and other amyloid proteins (Bronfman, P. C., et al., 1995, Neuroscience Lett. 218:201-203). In this assay, test compounds are incubated with a solution of Aβ (1-40) (20 μM) or IAPP (10 μM) containing 5 μM Thioflavin T, in 0.02M Tris/0.02M acetate/0.15M NaCl/0.005% azide/pH 7.40 at 37° C. in sealed 384 well microplates. Readings (ex 430 nm/em 485 nm) are taken at various time intervals with a microplate fluorescence reader. An increase in fluorescence signifies the appearance of amyloid or intermediates in the production of amyloid, as illustrated in the Figures (in general, a compound which inhibits fibrillogenesis produces lower fluorescence in the assay because the fluorescence of ThT is greater when bound to fibrils).

Protocol: Aβ peptide: Aβ (1-40) 95% purity (American Peptide Company, Inc, Sunnyvale, Cal. USA) is disaggregated in trifluoroacetic acid and filtered through a 0.02 μM filter, (Whatman Anotop 25 plus, 0.02 μm, Catalogue no. 6809 4102) in hexafluoroisopropanol (HFIP). Solutions of Aβ (1-40) or IAPP at 600 μm in HFIP are stored at −80° C. Assay mixture: The mixture is prepared as two solutions which are combined upon addition to the 384 well microplate (Corning Costar cat. 3705).

i) Solution A consists of test compounds in 0.02M Tris/0.02M acetate/0.15M NaCl/0.01% azide at pH 7.40 or buffer alone (control),

ii) Solution B consists of Aβ (1-40) 40 μM or IAPP 20 μM, Thioflavin T 10 mM in 0.02M Tris/0.02M acetate/0.15M NaCl at pH 7.40. This solution is prepared by drying the Aβ peptide under nitrogen and then resuspending this in 0.04M Tris base with 15 minutes sonication. An equal volume of 0.04M acetic acid containing 0.3 M NaCl is added and the solution is adjusted to 7.40±0.02. A small volume of 20 mM Thioflavin T is added to the solution to give a final 5 μM concentration of Thioflavin T.

iii) The microplate is loaded with 40 μL of solution A followed by 40 μL of solution B which gives a final 20 μM Aβ (1-40) or 10 μM IAPP, 5 μM Thioflavin T, and a given concentration of test compound in 0.02M Tris/0.02M acetate/0.15M NaCl/0.005% azide, pH 7.40.

The plate is sealed and loaded into the microplate fluorescence reader. Fluorescence measurement data analysis: The HTS-7000 Bio Assay Reader, Perkin Elmer, is used to perform kinetic runs of about 1 day. Readings were taken at 15 minute intervals, with one minute shaking before each read. Bandpass filters used were: excitation 430 nm, emission 485 mm.

Similarly, in the electron microscopy (“EM”) assay, each sample was sonicated for 1 min to disrupt large clumps before testing. The sample (5-μL aliquot) was placed on freshly cleaved mica and allowed to air dry. The mica was placed in a Balzers High-vacuum, Freeze-Etch Unit (model 301), shadowed with platinum (30° angle), and coated with a carbon film. The replica was removed from the mica by flotation and transferred onto a 300-mesh copper grid. Samples were examined using a Joel 2000 FX transmission electron microscope.

In the circular dichroism (“CD”) assay, samples were transferred to 0.1-cm path-length quartz cuvettes and CD scans were taken using a Jasco J-715 spectropolarimeter. Readings were taken at 37° C., between 190 and 240 nm, with a resolution of 0.1 nm and a bandwidth of 1 nm.

And in the mass spectroscopy (“MS”) assay, samples were prepared as aqueous solutions containing 20% ethanol, 200 μM of a test compound and 20 μM of solubilized Aβ40. The pH value of each sample was adjusted to 7.4 (±0.2) by addition of 0.1% aqueous sodium hydroxide. The solutions were then analyzed by electrospray ionization mass spectroscopy using a Waters ZQ 4000 mass spectrometer. Samples were introduced by direct infusion at a flow-rate of 25 μL/min within 2 hr. after sample preparation. The source temperature was kept at 70° C. and the cone voltage was 20 V for all the analysis. Data were processed using Masslynx 3.5 software. The MS assay gives data on the ability of compounds to bind to Aβ, whereas the ThT, EM and CD assays give data on inhibition of fibrillogenesis.

Some selected compounds of the present invention are presented in Table 2 below. Although particular salts are depicted (such as the hydrochloride), the free base and other pharmaceutically acceptable salts are within the present invention.

TABLE 2
Structures and Activities of Some Compounds of the Invention in Soluble Aβ Assays

| | Code | ThT | | | MS |
|---|---|---|---|---|---|
| Structure | No. | Assay | CD | EM | Assay |
| 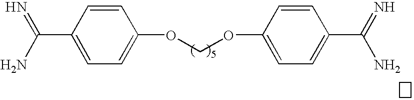 | 1 | + | | | + |
| 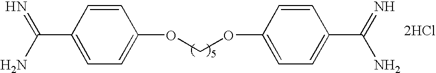 | 1 | + | + | + | + |
| 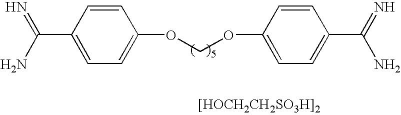 | 1 | + | | + | + |
| 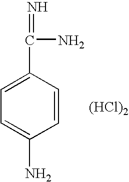 | 2 | | | | |
| 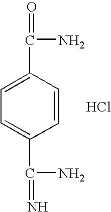 | 3 | | | | |
| 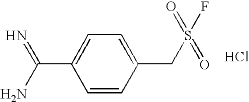 | 4 | | | | |
| 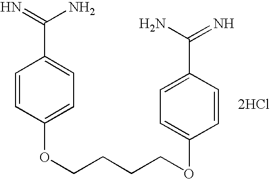 | 5 | + | | | + |
| 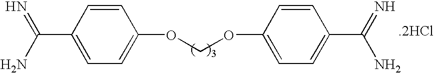 | 6 | + | + | − | + |
| 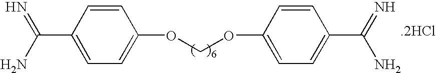 | 7 | + | + | + | + |
| 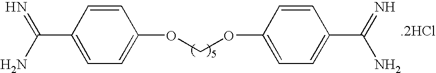 | 8 | + | + | − | + |
| 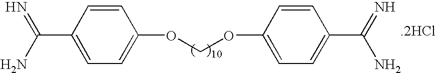 | 9 | + | + | − | + |
| 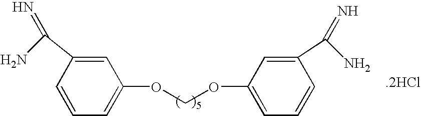 | 10 | + | | − | − |
| 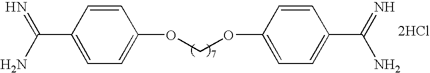 | 11 | + | + | + | + |
| 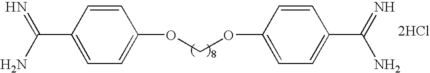 | 12 | + | + | + | + |
| 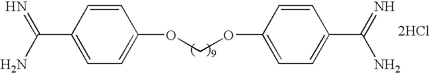 | 13 | + | + | + | + |
| 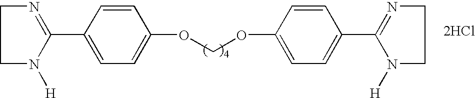 | 14 | + | + | − | − |
| 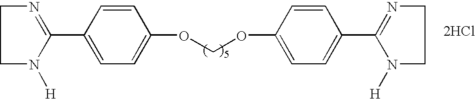 | 15 | + | + | − | − |
| 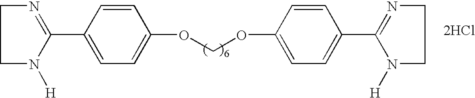 | 16 | + | + | − | + |
| 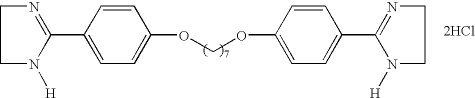 | 17 | + | + | − | + |
| 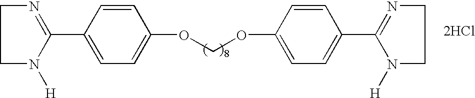 | 18 | + | + | − | + |
| 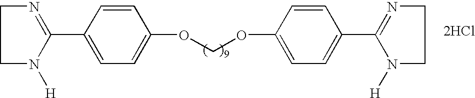 | 19 | + | + | | + |
| 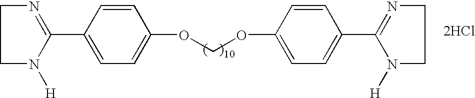 | 20 | nd | | − | + |
| 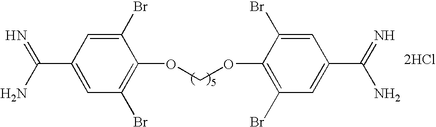 | 21 | pr | | | nd |
| 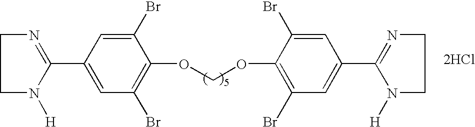 | 22 | pr | | | nd |
| 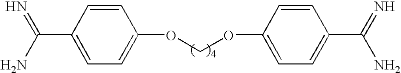 | 23 | + | | − | + |
| 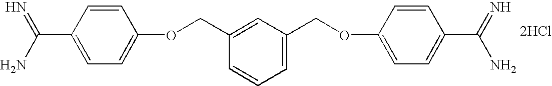 | 24 | + | | + | + |
| 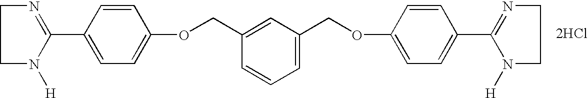 | 25 | + | | + | nd |
| 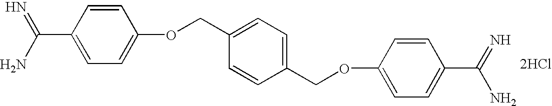 | 26 | + | | − | nd |
| 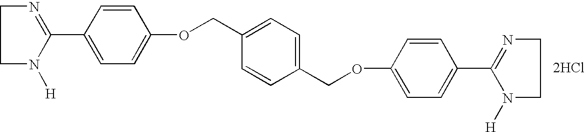 | 27 | pr | | | nd |
| 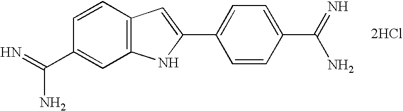 | 28 | + | + | + | − |
| 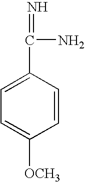 | 29 | | | | |
| 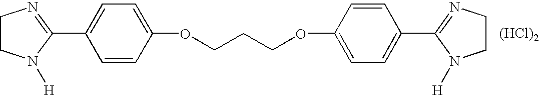 | 30 | | | | |
| 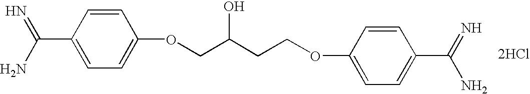 | 31 | + | | − | + |
| 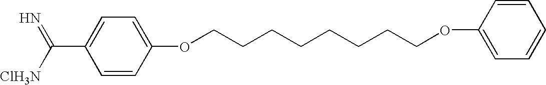 | 32 | | | | + |
| 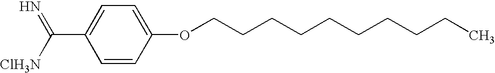 | 33 | | | | + |
| 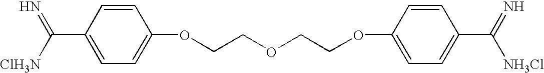 | 34 | + | | − | + |
| 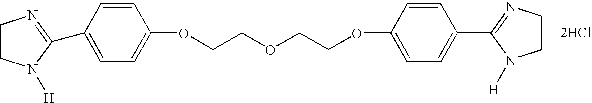 | 35 | + | | − | + |
| 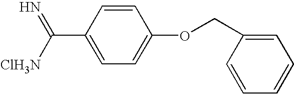 | 36 | + | | | + |
| 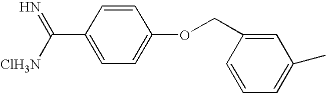 | 37 | + | | − | + |
| 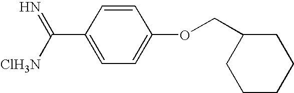 | 38 | + | | − | + |
| 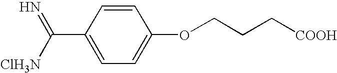 | 39 | + | | | + |
| 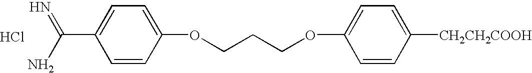 | 40 | | | | − |
| 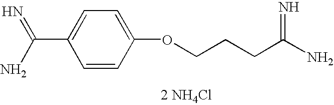 | 41 | | | | + |
| 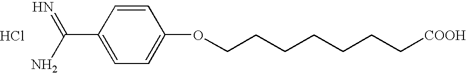 | 42 | | | | + |
| 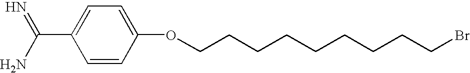 | 43 | | | | + |
| 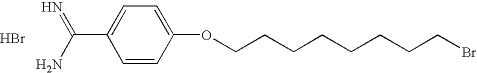 | 44 | | | | + |
| 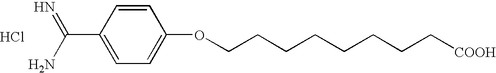 | 45 | | | | + |
| 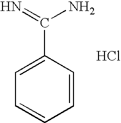 | 46 | − | − | − | nd |
| 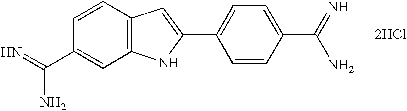 | 47 | | | | − |
| 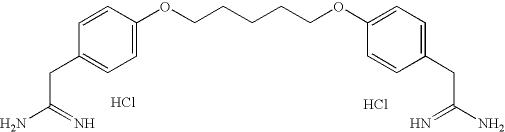 | 48 | | | | + |
| 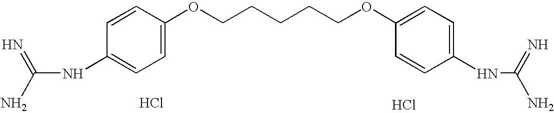 | 49 | | | | + |
| 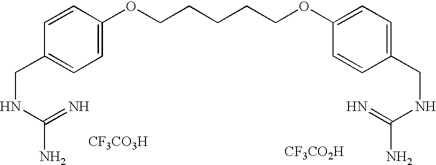 | 50 | | | | |
| 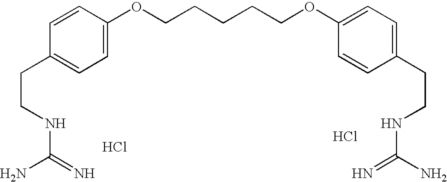 | 51 | | | | |
| 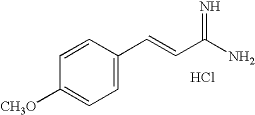 | 52 | | | | |
| 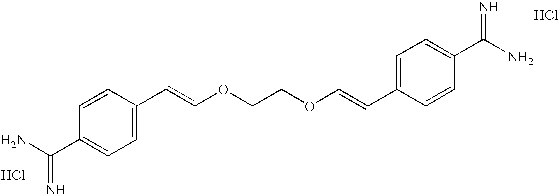 | 53 | | | | |
| 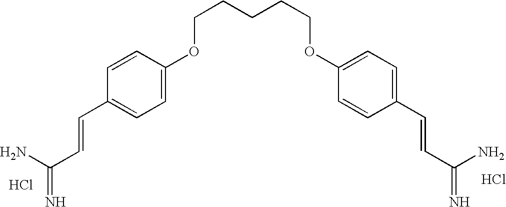 | 54 | | | | |
| 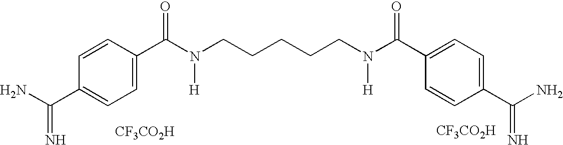 | 55 | | | | |
| 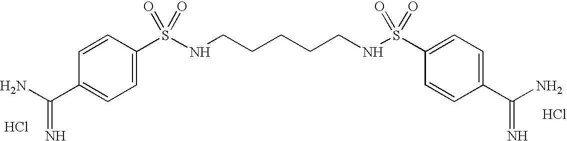 | 56 | | | | |
| 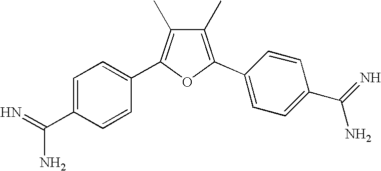 | 57 | | | | + |
| 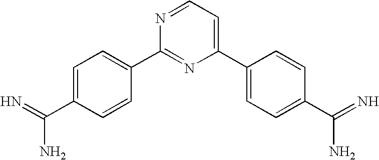 | 58 | | | | + |
| 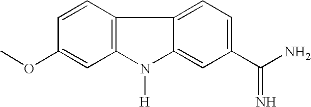 | 59 | | | | + |
| 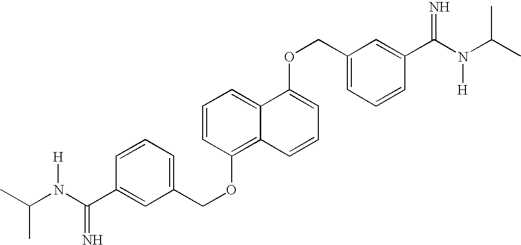 | 60 | | + | − | + |
| 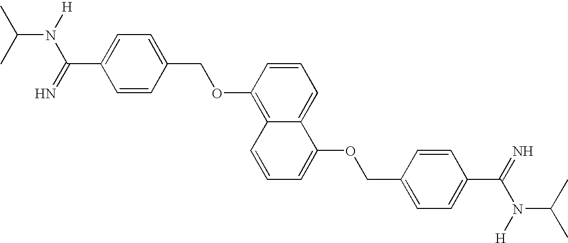 | 61 | | + | + | + |
| 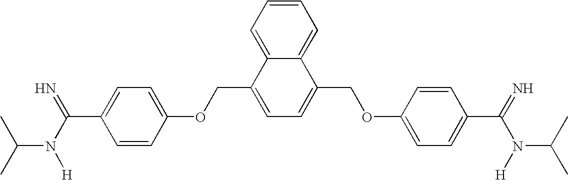 | 62 | | + | + | + |
| 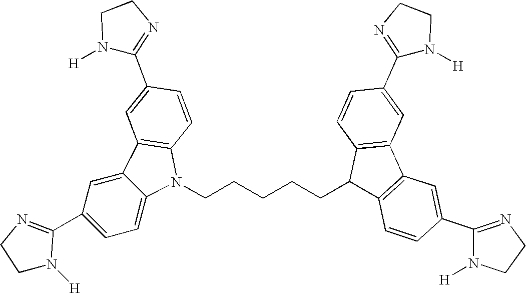 | 63 | | | | + |
| 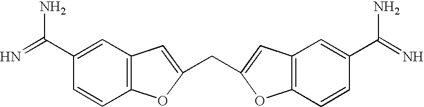 | 64 | | | | + |
| 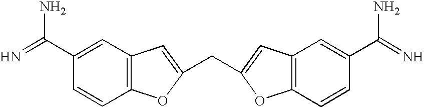 | 64 | | | | + |
| 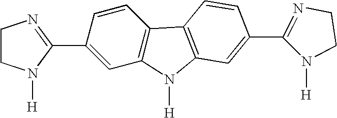 | 65 | | − | + | + |
| 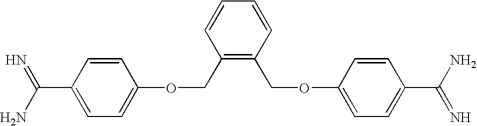 | 66 | | | | + |
| 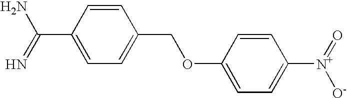 | 67 | | | | − |
| 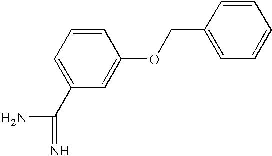 | 68 | | | | − |
| 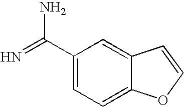 | 69 | | | | − |
| 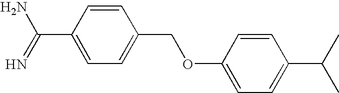 | 70 | | + | + | + |
| 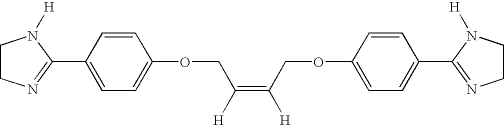 | 71 | | | | + |
| 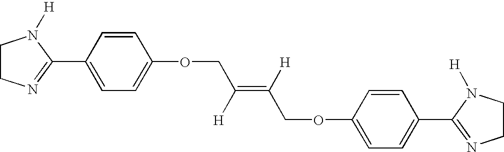 | 72 | | | | + |
| 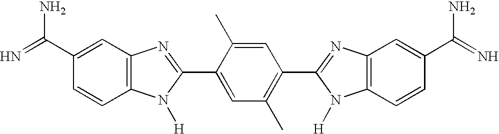 | 73 | | | | + |
| 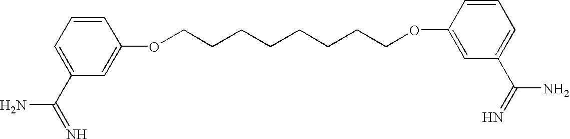 | 74 | | − | + | + |
| 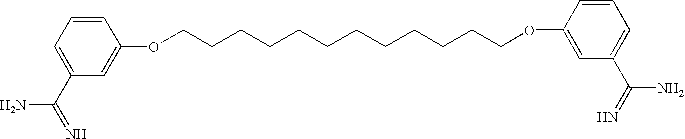 | 75 | | − | + | + |
| 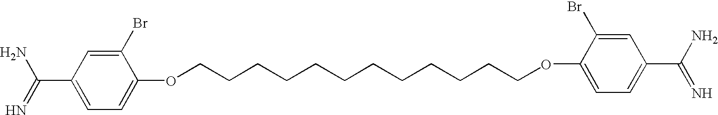 | 76 | | − | + | + |
| 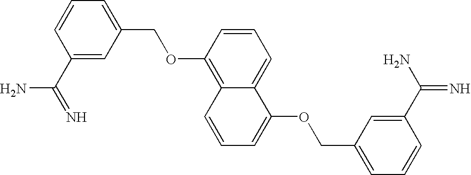 | 77 | | | | + |
| 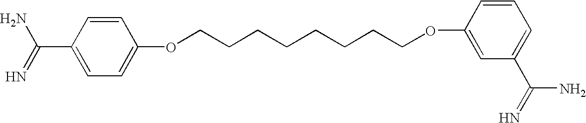 | 78 | | − | + | + |
| 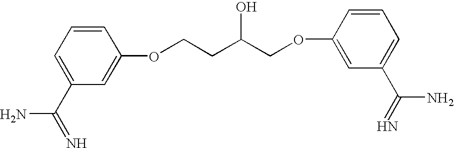 | 79 | | | | + |
| 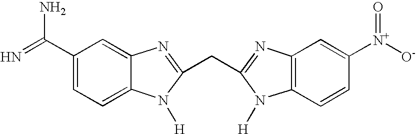 | 80 | | | | + |
| 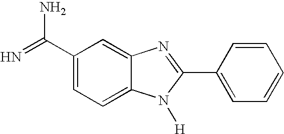 | 81 | | | | + |
| 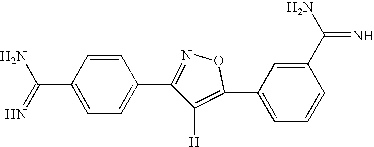 | 82 | | | | − |
| 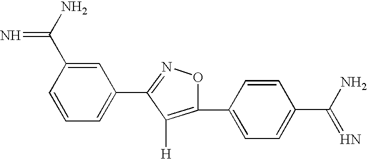 | 83 | | − | + | + |
| 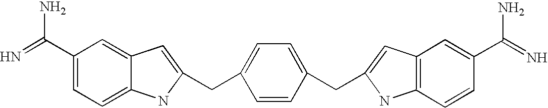 | 84 | | | | + |
| 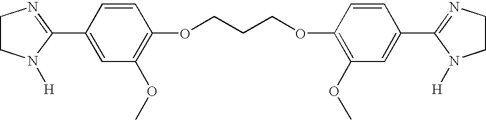 | 85 | | | | − |
| 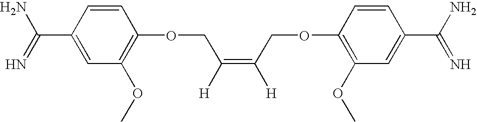 | 86 | | | | + |
| 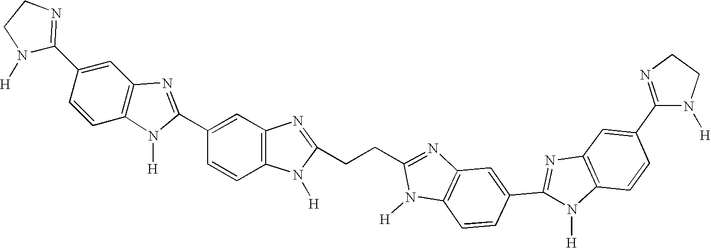 | 87 | | | | + |
| 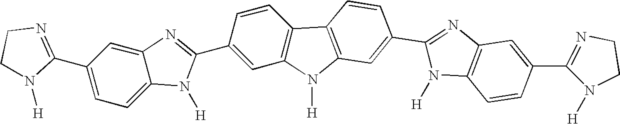 | 88 | | | | + |
| 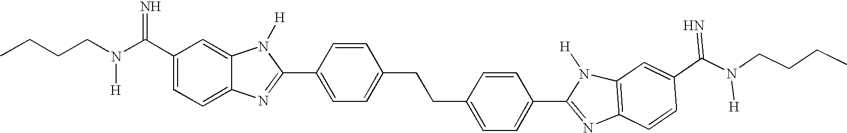 | 89 | | | | + |
| 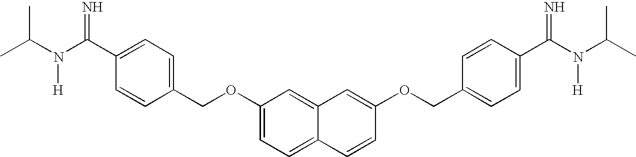 | 90 | | | | + |
| 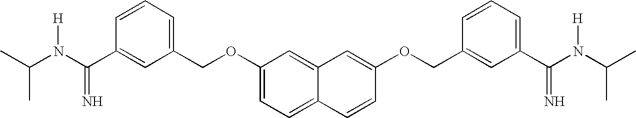 | 91 | | | | + |
| 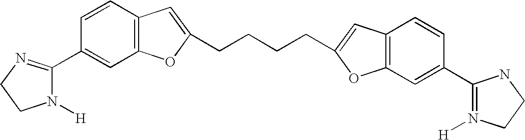 | 92 | | + | + | + |
| 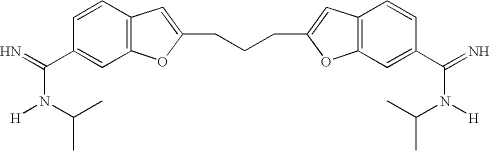 | 93 | | | | − |
| 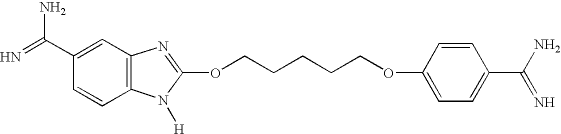 | 94 | | | | + |
| 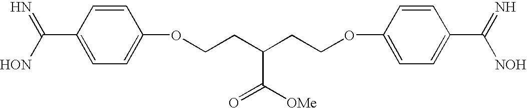 | 95 | | | | + |
| 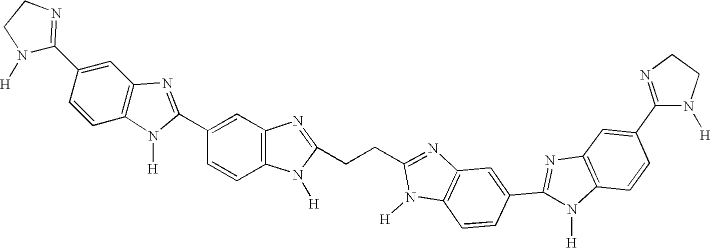 | 87 | | | | + |
| 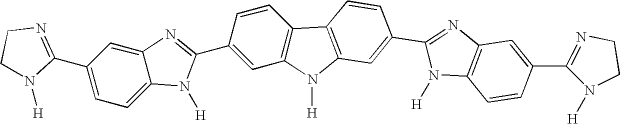 | 88 | | | | + |
| 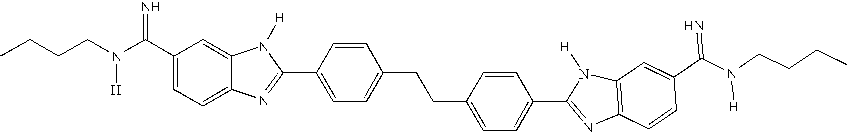 | 89 | | | | + |
| 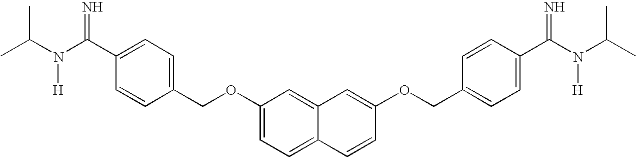 | 90 | | | | + |
| 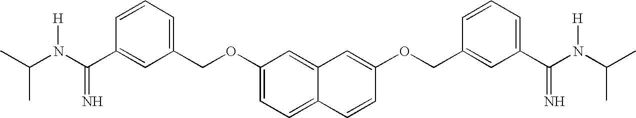 | 91 | | | | + |
| 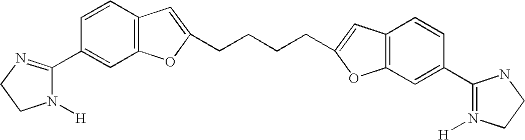 | 92 | | + | + | + |
| 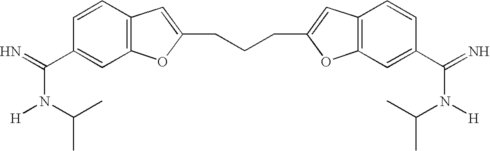 | 93 | | | | − |
| 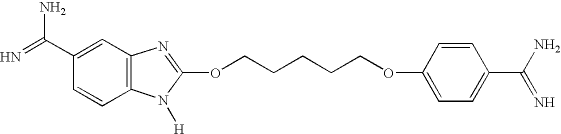 | 94 | | | | + |
| 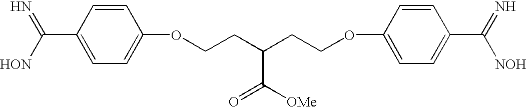 | 95 | | | | + |
| 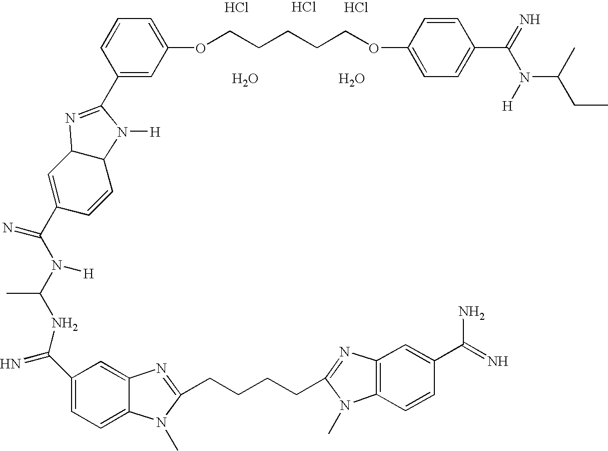 | 96 97 | | | | ++ |
| 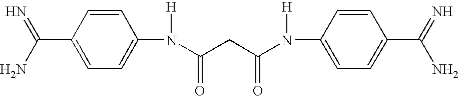 | 98 | | | | + |
| 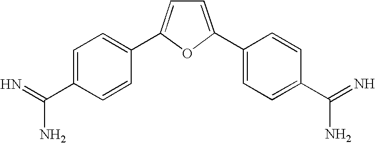 | 99 | | | | + |
| 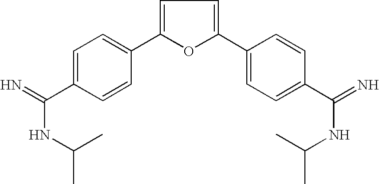 | 100 | | | | + |
| 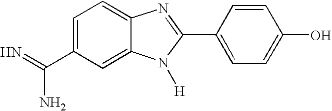 | 101 | | | | + |
| 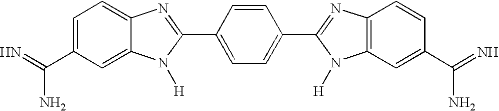 | 102 | | | | + |
| 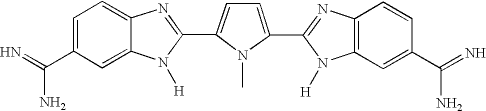 | 103 | | − | + | + |
| 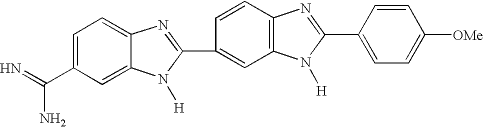 | 104 | | | | + |
| 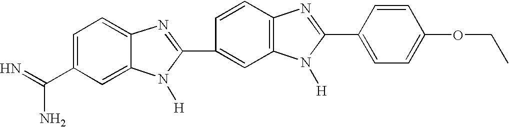 | 105 | | | | + |
| 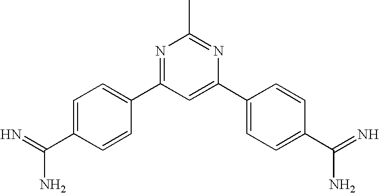 | 106 | | | | + |
| 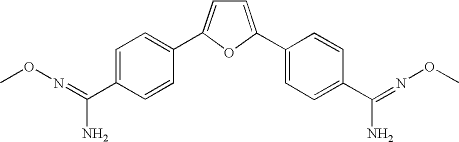 | 107 | | | | − |
| 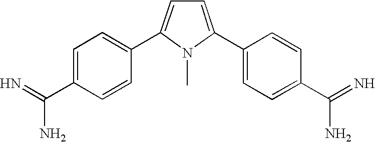 | 108 | | | | + |
| 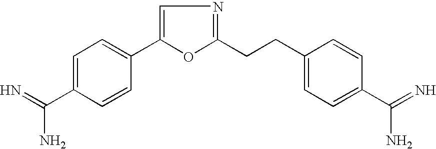 | 109 | | | | + |
| 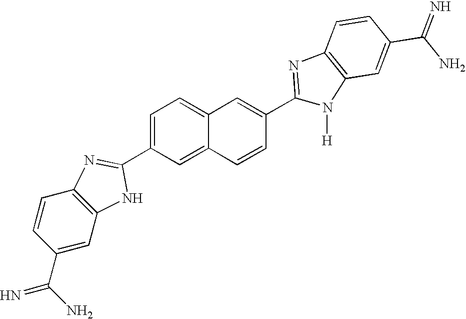 | 110 | | | | + |
| 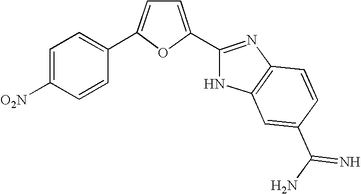 | 111 | | | | + |
| 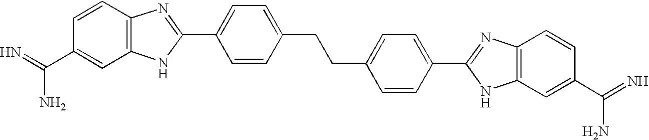 | 112 | | − | + | + |
| 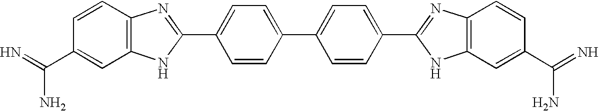 | 113 | | | | + |
| 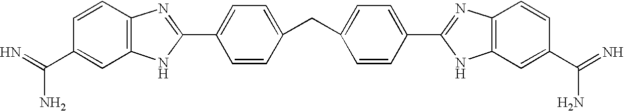 | 114 | | − | + | + |
| 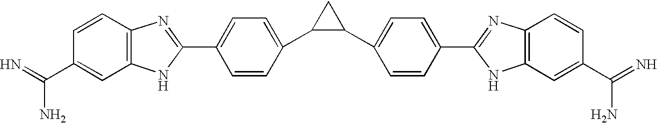 | 115 | | − | + | + |
| 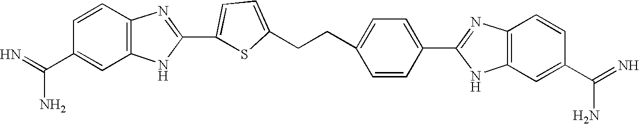 | 116 | | − | + | + |
| 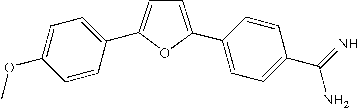 | 117 | | − | + | + |
| 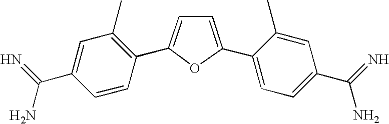 | 118 | | | | − |
| 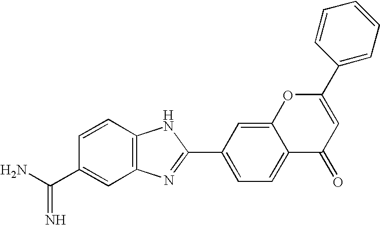 | 119 | | | | + |
| 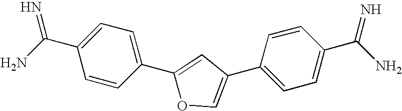 | 120 | | − | + | + |
| 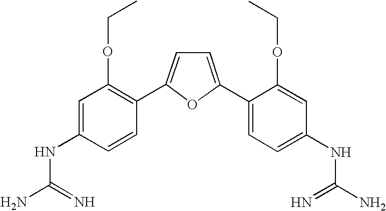 | 121 | | − | + | + |
| 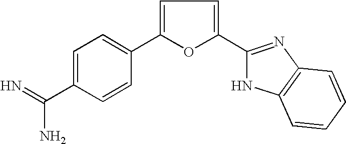 | 122 | | + | + | + |
| 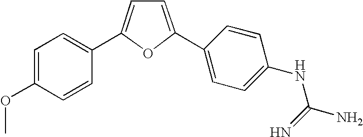 | 123 | | − | + | + |
| 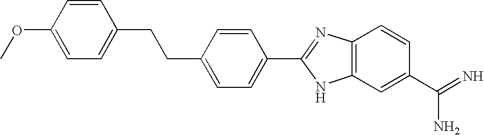 | 124 | | − | + | + |
| 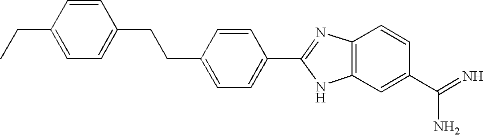 | 125 | | − | + | + |
| 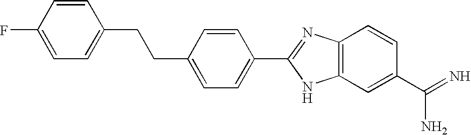 | 126 | | | | + |
| 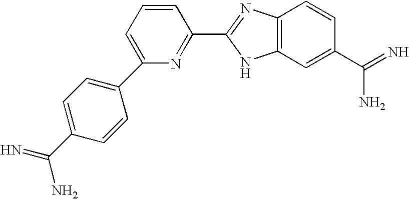 | 127 | | | | + |
| 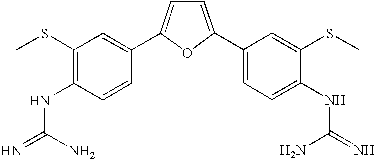 | 128 | | + | + | + |
| 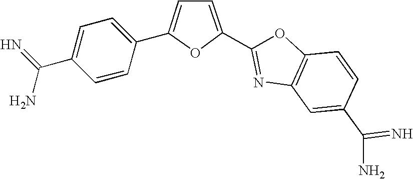 | 129 | | | | + |
| 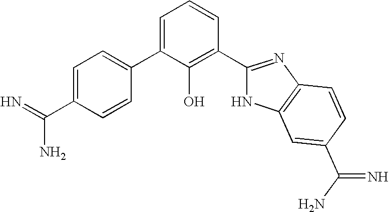 | 130 | | | | + |
| 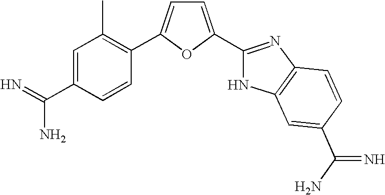 | 131 | | | | + |
| 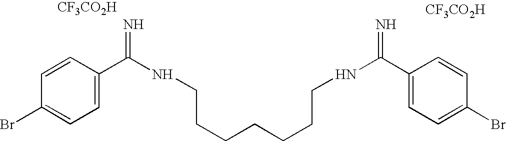 | 132 | | | | |
| 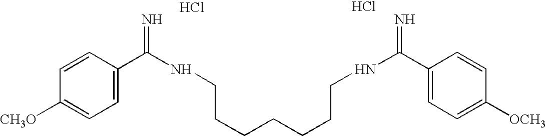 | 133 | | | | |
| 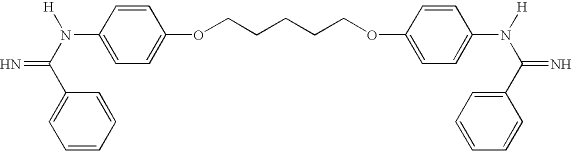 | 134 | | + | − | + |
| 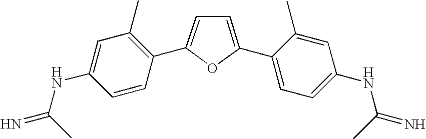 | 135 | | | | + |
| 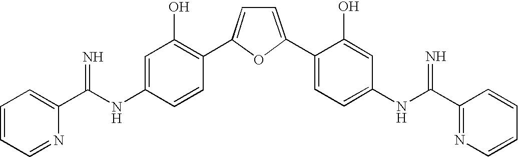 | 136 | | | | + |
| 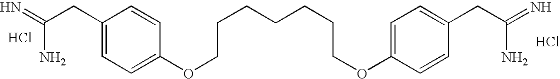 | 137 | | | | |
| 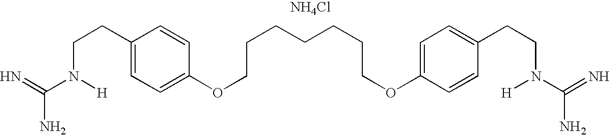 | 138 | | | | |
| 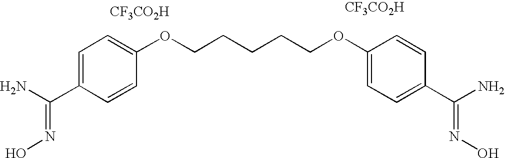 | 139 | | | | |
| 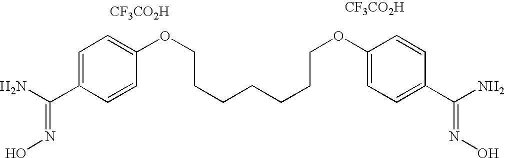 | 140 | | | | |
| 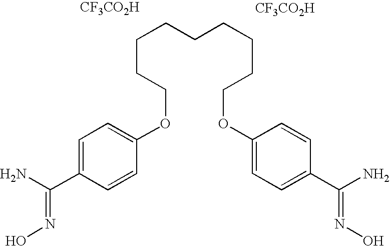 | 141 | | | | |
| 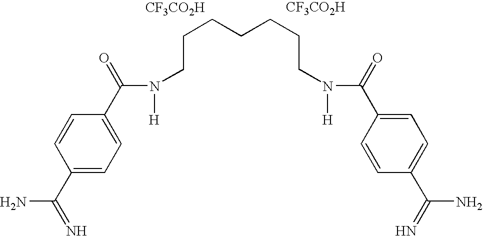 | 142 | | | | |
| 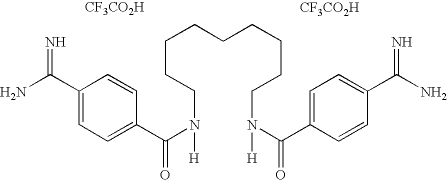 | 143 | | | | |
| 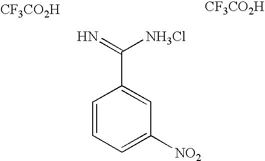 | 144 | | | | |

In each assay, “+” = active; “−” = inactive; “pr” = promoting; “nd” = or blank entry = not determined.

The following compounds in Table 3 may also be employed according to the methods described herein.

TABLE 3
Additional Exemplary Compounds for Use in The Methods of The Invention

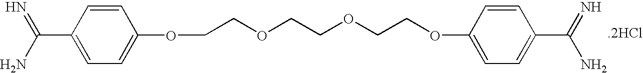

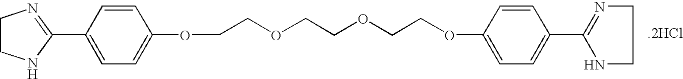

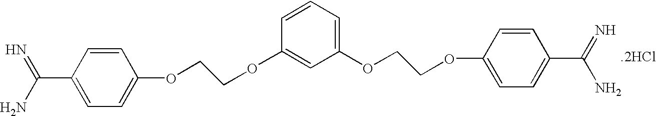

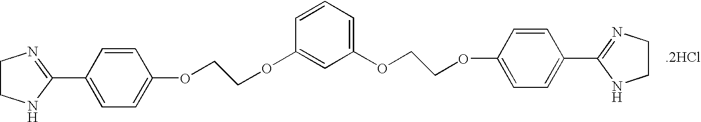

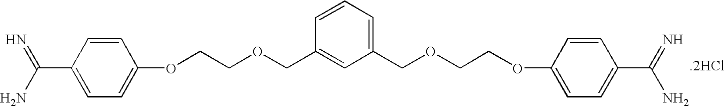

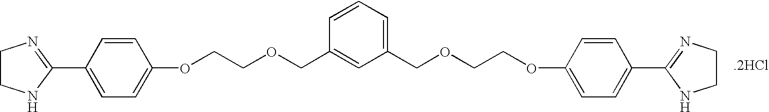

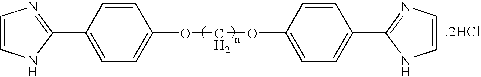

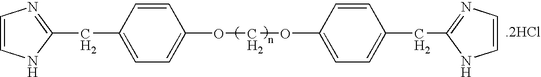

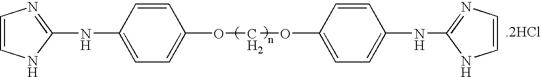

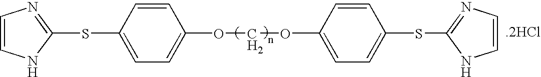

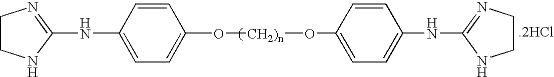

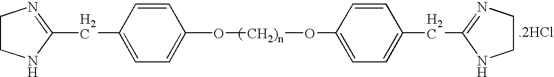

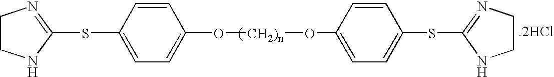

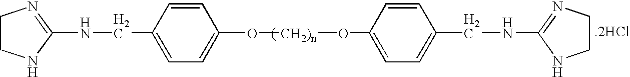

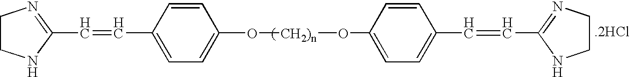

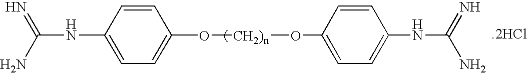

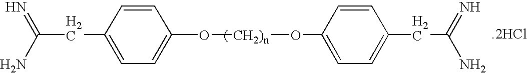

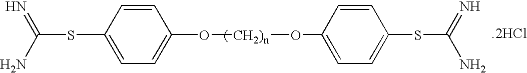

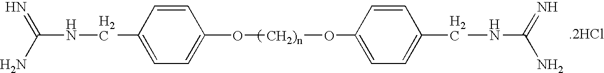

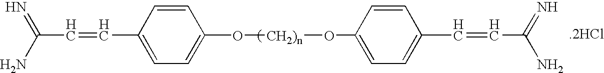

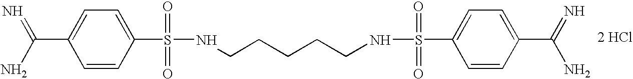

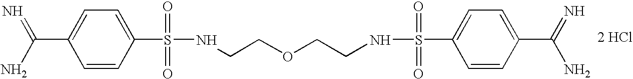

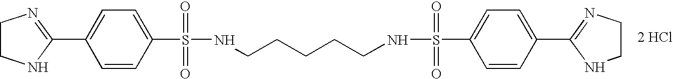

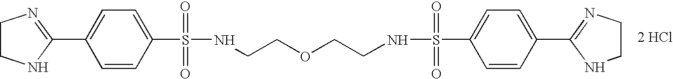

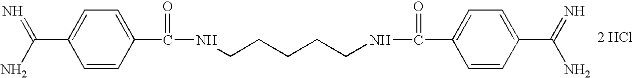

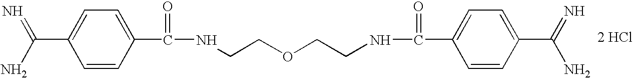

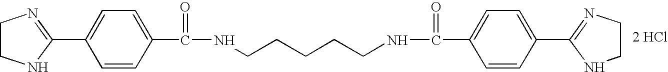

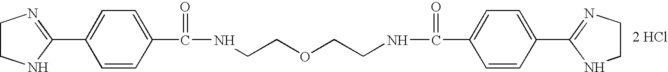

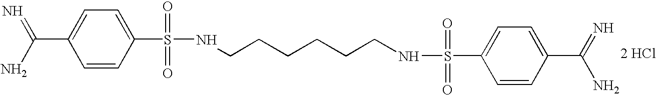

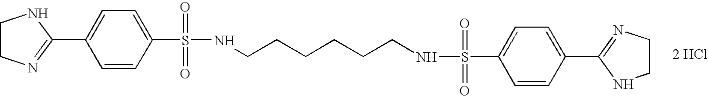

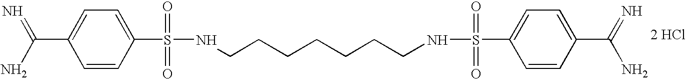

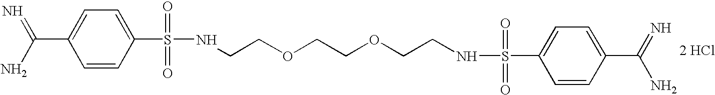

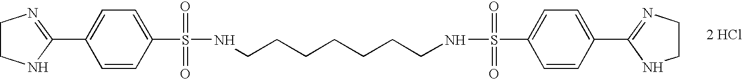

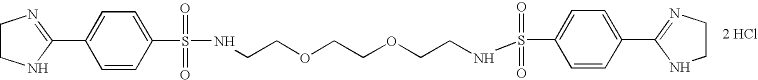

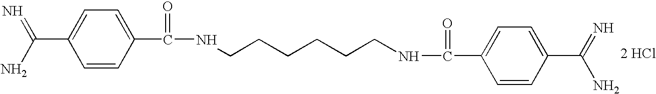

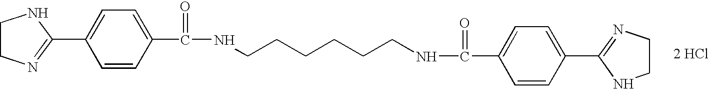

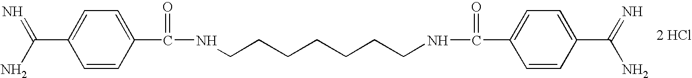

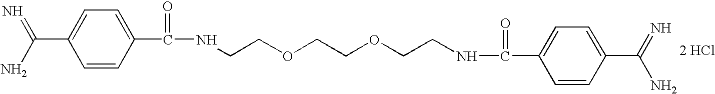

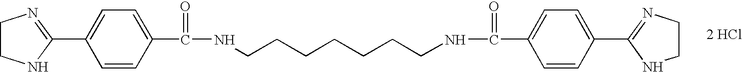

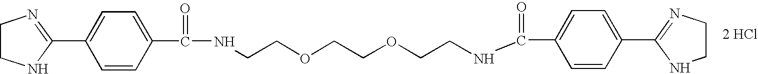

The following charts are results from the ThT assay.

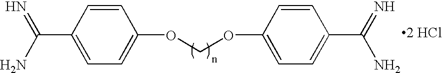

[image]

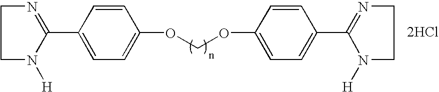

[image]

The present invention also relates to novel compounds and the synthesis thereof. Accordingly, the following examples are presented to illustrate how some of those compounds may be prepared.

General Aspects

Chemicals were purchased from Aldrich. Analytical thin-layer chromatography (TLC) was performed on silica gel 60 F₂₅₄ plastic-backed plates. Solvents were reagent grade unless otherwise specified. The ¹H (500 MHz) and ¹³C (125 MHz) were recorded on a Varian Inova 500. The chemical shifts are reported on the 6 scales in parts per million (ppm). The infra-red (IR) spectra were carried out on a Perkin-Elmer Spectra One spectrometer (neat compound on NaCl plate).

### 1,4-bis(4-amidinoanilino)butane

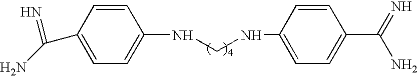

### Step 1: 1,4-bis(4-cyanoanilino)butane

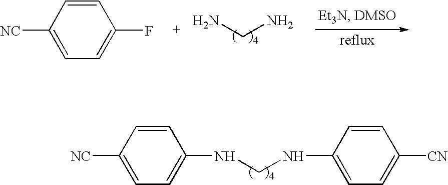

A mixture of 4-fluorobenzonitrile (3 g, 0.025 mol), 1,4-diaminobutane (0.6 g, 0.006 mol), triethylamine (5 mL) and DMSO (16 mL) was heated at 150° C. with stirring for 3 h. The mixture was then poured into iced water (250 mL) and the precipitate was collected by filtration. Recrystallization of the crude product (0.58 g) from DMSO/H₂O (6:1) gave the product as a light yellow solid, 0.48 g, yield 27.6%.

### Step 2: 1,4-bis(4-amidinoanilino)butane

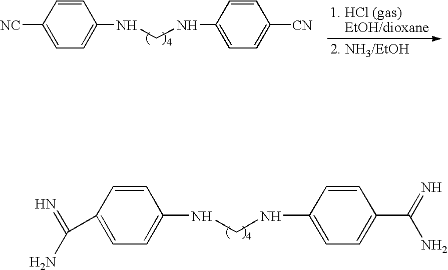

A mixture of 1,4-bis(4-cyanoanilino)butane (0.44 g, 1.52 mmol) in ethanol (30 mL) and dioxane (10 mL) was cooled to 0° C. and saturated with HCl gas. The resulting mixture was stirred at room temperature until the IR indicated the disappearance of the nitrile absorbance peak at ˜2200 cm⁻¹. Diethyl ether (100 mL) was added, and the formed precipitate was collected and washed with diethyl ether. The solid thus obtained was then placed into a 50-mL round bottom flask. Ethanolic ammonia (2 M, 30 mL) was slowly added via syringe. The resulting mixture was refluxed for 3 h, and then was cooled to room temperature. Diethyl ether (100 mL) was added to induce precipitation. The precipitate thus formed was collected, washed with ether, and recrystallized from H₂O to give 0.50 g of product, yield 99%.
Linear dibenzamidine and diimidazolino compounds

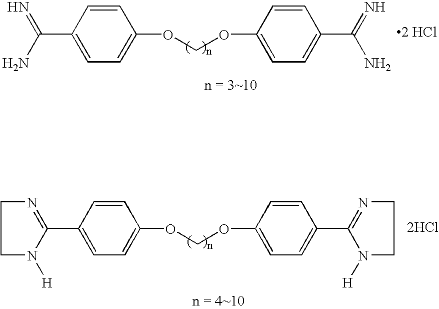

### Step 1: α, ω-bis(4-cyanophenoxy)alkanes

Sodium (1.2 g, 0.05 mol) was cut into small pieces and slowly added to a stirred solution of dry ethanol (40 mL). After complete dissolution of the sodium, 4-cyanophenol (6 g, 0.05 mol) was added and followed by the dropwise addition of 1,4-dibromobutane (5.4 g, 0.025 mol). The resulting mixture was stirred at reflux for 1-2 days and then cooled to room temperature. The white solid formed in the reaction was collected by vacuum filtration, washed with water and dried under vacuum. The obtained product, 1,4-bis(4-cyanophenoxy)butane (7.18 g, 98% yield), was used directly for the next step without purification. Analogous compounds with n=3, 5, 6, 7, 8, 9, and 10 were prepared and yields ranged from 70-95%. The ¹H and ¹³C NMR of the compounds were consistent with the structures.

### Step 2: Dibenzamidines and diimidazolino compounds

A mixture of α, ω-bis(4-cyanophenoxy)alkane (3.42 mmol), dioxane (15 mL) and ethanol (40 mL) was cooled to 0° C. Dry HCl gas was bubbled through the mixture until saturation. The mixture was stirred at room temperature until the IR nitrile absorbance at 2200 cm⁻¹ subsided.

Diethyl ether (100 mL) was then added and a white precipitate was formed. The precipitate was collected by vacuum filtration, washed with diethyl ether, and placed into a 50-mL round bottom flask. Ethanolic ammonia solution (2 M, 30 mL; in the preparation of dibenzamidines) or ethylenediamine in MeOH (1.5 M, 30 mL; in the preparation of diimidazolino compounds) was added slowly via syringe. The resulting mixture was stirred at reflux for 3 h. After the mixture was cooled to room temperature, diethyl ether (100 mL) was added. The white precipitate that formed was collected and washed with diethyl ether. The solid was then recrystallized with HCl (2 N) giving the desired product. Dibenzamidine compounds with n=3-10 were prepared and yields ranged from 60-85%. Diimidazolino compounds with n=4-10 were prepared and yields ranged from 50-92%.

### 1-(4-amidino)phenoxy-8-bromooctane, hydrobromide

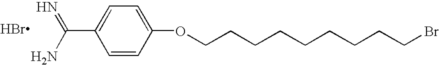

### Step 1: 1-(4-cyano)phenoxy-8-bromooctane

In a 100-mL round-bottom flask were placed 4-cyanophenol (2.38 g, 20 mmol), K₂CO₃ (anhydrous, 25 mmol) and DMF (50 mL). The mixture was stirred at room temperature for 30 min. When the mixture became cloudy, 8-bromooctanol (20 mmol) was added dropwise via syringe. The mixture was then refluxed for 5 h, cooled to room temperature, and poured into iced water (200 mL). White precipitate was formed and collected by vacuum filtration. The pure product (4.1 g, 88.7% yield) was obtained as a white solid after silica gel flash column chromatography (eluent: 20-40% ethyl acetate in hexane).

### Step 2: 8-(4-amidinophenoxy)octanol

The corresponding amidine compounds were obtained by serial treatments with saturated ethanolic hydrochloride solution and ethanolic ammonia analogously as described above.

### Step 3: 1-(4-amidinophenoxy)-8-bromooctane, hydrobromide

In a 50-mL round-bottom flask were placed 8-(4-amidinophenoxy)octanol (2.14 g, 6.8 mmol) and dichloromethane (30 mL). The mixture was cooled to 0° C., and PBr₃ (3.4 mmol, 0.5 eq.) was added dropwise via syringe. Then the mixture was stirred overnight at room temperature. The white solid starting material gradually dissolved and turned into a yellow oil phase immiscible with the dichloromethane. Upon completion of the reaction, water was added to quench the reaction, and the dichloromethane was evaporated under reduced pressure to give a white solid as crude product. Pure product (white solid, 780 mg, 31% yield) was obtained after silica gel flash column chromatography (eluent CHCl₃/MeOH/AcOH 94/5/1) and subsequent recrystallization from HBr/CH₃CN (2 N).

### 9-(4-amidinophenoxy)nonanoic acid, hydrochloride

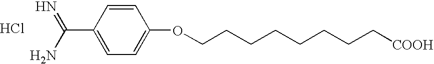

### Step 1: 9-(4-cyanophenoxy)nonanol

In a 100-mL round-bottom flask, 4-cyanophenol (2.38 g, 20 mmol) and K₂CO₃ (anhydrous, 25 mmol) were mixed in DMF (50 mL). The mixture was stirred at room temperature for 30 min. When the mixture became cloudy, 9-bromononanol (20 mmol) was added dropwise via syringe. The mixture was then refluxed for 5 h, cooled to room temperature, and poured into iced water (200 mL). The white precipitate that formed was collected by vacuum filtration. The pure product (4.8 g, 98% yield) was obtained as a white solid after silica gel flash column chromatography (eluent: 20-40% ethyl acetate in hexane).

### Step 2: 9-(4-cyanophenoxy)nonanoic acid

To a solution of 9-(4-cyanophenoxy)nonanol (2.5 g, 10.2 mmol) in DMF (50 mL), PDC (19 g, 61 mmol, 6 eq.) was added. The mixture was stirred at 50° C. overnight, then cooled to room temperature, and poured into iced water (150 mL). The mixture was extracted with ethyl acetate (4×50 mL). The combined organic layers were washed with brine and dried over sodium sulfate. Purification via silica gel flash column chromatography (eluent 25-50% ethyl acetate in hexane) gave product as a white solid, 1.65 g, 62% yield.

### Step 3: 9-(4-cyanophenoxy)nonanoic acid, ethyl ester

In a 100-mL round-bottom flask, thionyl chloride (0.88 mL, 12 mmol) was added to anhydrous ethanol (50 mL). The mixture was stirred for 10 min, then 9-(4-cyanophenoxy)nonanoic acid (1.65 g, 6.02 mmol) was added in one portion. The reaction was monitored by TLC. Upon completion of the reaction, ethanol was removed under reduced pressure. Ether (100 mL) and saturated sodium bicarbonate solution (100 mL) was added. The organic phase was separated and dried over sodium sulfate. The product (1.6 g, 87.7% yield) was obtained as a white solid after evaporation of the solvent.

### Step 4: 9-(4-amidinophenoxy)nonanoic acid hydrochloride

9-(4-cyanophenoxy)nonanoic acid ethyl ester (1.6 g, 5.28 mmol) was dissolved in a mixture of ethanol and dioxane (50/10 mL) in a sealed 100-mL round-bottom flask. The mixture was saturated with HCl (g) at 0° C. and stirred at room temperature until IR showed the disappearance of the nitrile absorbance at 2200 cm⁻¹. Ethanol/dioxane was then removed under reduced pressure, and ether (100 mL) was added to induce precipitation. The precipitate was collected and immediately placed into a dry 100-mL flask. Ethanolic ammonia (2 M, 40 mL) was added via syringe. The mixture was refluxed for 3 h, followed by removal of the solvent and addition of ether to induce precipitation. The solid that formed was collected and recrystalllized from HCl (2 N). Final product was obtained as a colorless needle crystal, 0.56 g, 32.3%. ¹H NMR (500 MHz, DMSO-d₆): 11.96 (s, 1H), 9.16 (s, 2H), 8.85 (s, 2H), 7.80 (d, 2H, J=8.5 Hz), 7.13 (d, 2H, J=8.5 Hz), 4.06 (t, 2H, J=6.5 Hz), 2.18 (t, 2H, J=7.5 Hz), 1.73-1.70 (m, 2H), 1.49-1.46 (m, 2H), 1.40-1.38 (m, 2H), 1.30-1.27 (m, 6H); ¹³C NMR (125 MHz, DMSO-d₆): 174.46, 164.71, 163.08, 130.15, 119.23, 114.74, 68.08, 33.66, 28.66, 28.57, 28.47, 28.40, 25.34, 24.46.
Some Substituted Pentamidines

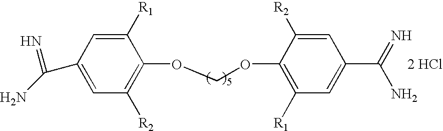

### Step 1: 1,5-Bis(4-cyano-2-methoxyphenoxy)pentane

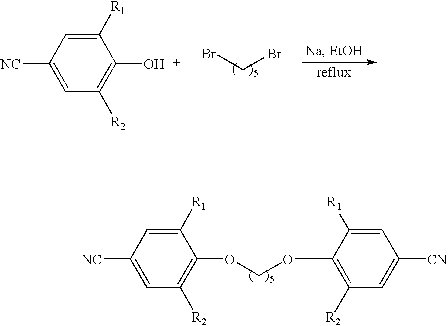

Sodium (0.3 g, 0.014 mol) was cut into small pieces and slowly added to a stirred solution of dry ethanol (30 mL). After complete dissolution of the sodium, 4-hydroxy-3-methoxybenzonitrile (2 g, 0.013 mol) was added and followed by the dropwise addition of 1,5-dibromopentane (0.9 mL, 0.007 mol). The resulting mixture was stirred at reflux for 2 days, and then cooled to room temperature. The light brown precipitate in the mixture was collected, washed with water and dried under vacuum. The product obtained (1.45 g, 73%) was used directly for the next step without purification. The ¹H and ¹³C NMR of the compounds were consistent with the structures.

### Step 2: Corresponding Pentamidines

A mixture of substituted 1,5-bis(4-cyanophenoxy)pentane (in this example, R₁=methoxy and R₂=hydrogen) (1.8 g, 4.91 mmol), dioxane (15 mL) and ethanol (50 mL) was cooled to 0° C. Dry HCl gas was bubbled through the mixture until saturation. The mixture was stirred at room temperature until IR showed the disappearance of the nitrile absorbance at 2200 cm⁻¹. Then diethyl ether (100 mL) was added and the white precipitate that formed was collected by vacuum filtration and washed with diethyl ether.

The white solid obtained was placed into a 50-mL round-bottom flask and ammonia ethanol solution (2 M, 30 mL) was added slowly via syringe. The resulting mixture was stirred at reflux for 3 h. After the mixture was cooled to room temperature, diethyl ether (100 mL) was added and a white precipitate formed. The precipitate was collected and washed with diethyl ether. The solid was then recrystallized from 2 N HCl giving the desired product (0.92 g, 40% yield). In like manner, the corresponding compound with R₁=bromine and R₂=bromine was synthesized in 53% yield.

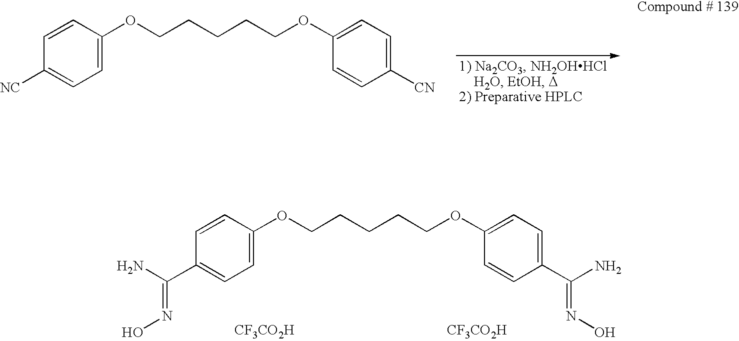

A mixture of 1,5-bis(4-cyanophenoxy)pentane (153 mg, 0.5 mmol), sodium carbonate (180 mg, 1.7 mmol) and hydroxylamine hydrochloride (278 mg, 4 mmol) in 80% ethanol (10 mL) was heated at reflux for 2 h. The mixture was cooled to room temperature. Some solid precipitated and was removed by filtration. The filtrate was concentrated to dryness under reduced pressure. The crude product was purified by preparative RP-HPLC (Vydac C18, 215 nm, 50 mL/min, 0% to 90% MeCN in H₂O containing 0.1% TFA) and lyophilized to give a white solid, 127.2 mg, 42%. The heptane and nonane anolags were prepared in the same way.

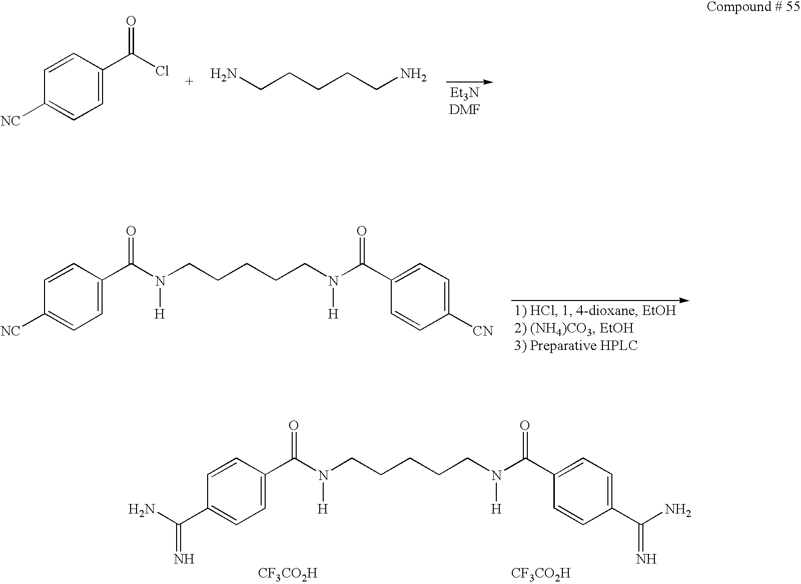

Step 1: To a cold solution (0° C.) of 1,5-diaminopentane (0.35 mL, 3 mmol) and triethylamine (0.98 mL, 7 mmol) in DMF (10 mL) was added 4-cyanobenzoyl chloride (1 g, 6 mmol). The mixture was stirred overnight at room temperature, and then diluted with water. The beige solid that precipitated was collected by filtration and dried in vacuo, giving the corresponding amide, 1 g, 92%.

Step 2: A suspension of the 1,5-bis-(4-cyanobenzamido)pentane (465 mg, 1.3 mmol), in a mixture of absolute ethanol (25 mL) and 1,4-dioxane (20 mL), was cooled to 0° C., saturated with dry HCl, and the resulting mixture was stirred for 60 hours at room temperature. The solvent was evaporated under reduced pressure. A brownish solid was obtained. A mixture of the solid and ammonium carbonate (2.5 g, 25 mmol) in ethanol (25 mL) was stirred overnight at room temperature. A small amount of activated charcoal was added, then the mixture was filtered over Celite. The solvent was evaporated under reduced pressure. The crude product was purified by preparative RP-HPLC (Vydac C18, 215 nm, 50 mL/min, 0% to 90% MeCN in H₂O containing 0.1% TFA) and lyophilized to give the title compound as a white solid, 410 mg, 51%. The heptane and nonane analogs were prepared in the same way.

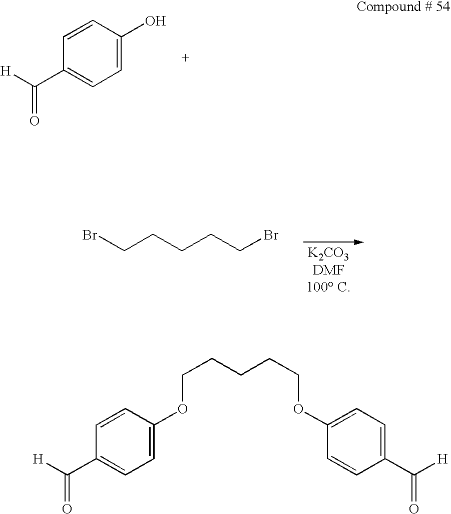

Step 1: A mixture of 4-hydroxybenzaldehyde (2.7 g, 22 mmol), 1,5-dibromopentane (1.35 mL, 10 mmol) and potassium carbonate (5.2 g) in dry DMF (25 mL) was heated at 100° C. with an oil bath for 5 hours. The mixture was cooled to room temperature, then water (100 mL) was added. The solid that formed was collected by filtration, rinsed with water and dried in vacuo. The desired bis-aldehyde was obtained as a brownish solid, 2.8 g, 89%.

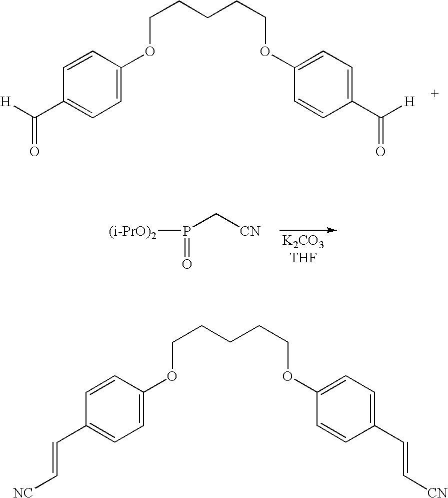

Step 2: Diisopropyl (cyanomethyl)phosphonate (0.86 mL, 4.2 mmol) was added to suspension of sodium hydride (4.4 mmol) in THF at (0° C.). The mixture was stirred at room temperature for 1 hour. A solution of the bis-aldehyde (2 mmol) in THF was added. The mixture was stirred at room temperature for 2 h, then diluted with ethyl acetate, washed subsequently with water, saturated sodium bicarbonate, brine and dried over magnesium sulfate. The solvent was evaporated under reduced pressure. The crude solid was washed with a mixture of ethyl acetate and hexane (1 to 10, 10 mL) the dried in vacuo to afford the bis-nitrile, 0.51 g, 71% yield.

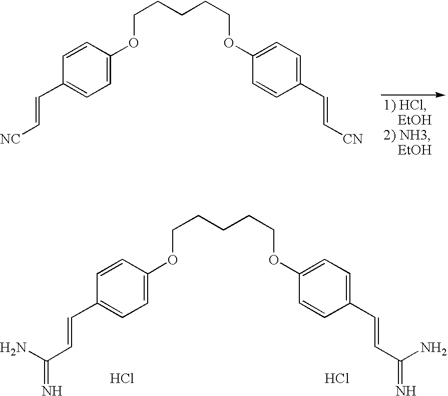

Step 3: A suspension of the bis-nitrile (0.48 g, 1.34 mmol) in ethanol (20 mL) was saturated with HCl at 0° C. The mixture was stirred at room temperature for 3 days. The solvent was evaporated under reduced pressure. The solid was then dissolved in 2 N NH₃ in ethanol (20 mL) and the mixture was heated at reflux for 2 h. The mixture was cooled to room temperature and the solvent was evaporated under reduced pressure. The resulting solid was dried in vacuo, then recrystallized from 2 N HCl with the addition of a few drops of ethanol. The solid was collected by filtration, rinsed with water and dried overnight in vacuo, giving the title compound as a light yellow solid, 0.44 g, 71%.

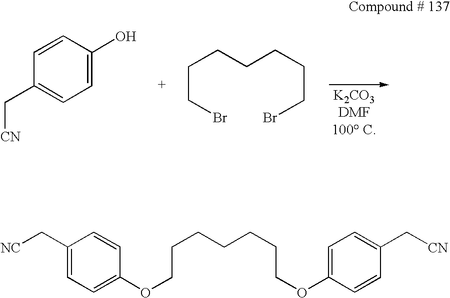

Step 1: A mixture of 4-hydroxybenzylcyanide (2.56 g, 19.2 mmol), 1,7-dibromoheptane (1.49 mL, 8.7 mmol), potassium carbonate (11 g) in DMF (30 mL) was heated with an oil bath at 100° C. for 3 hours. The mixture was cooled to room temperature and diluted with water (150 mL). A solid precipitated. The solid was collected by filtration and rinsed with water. It was then dissolved in ethyl acetate, washed subsequently with 10% NaOH (3×20 mL), brine (30 mL) and dried over magnesium sulfate. The solvent was evaporated under reduced pressure. The resulting solid was dried in vacuo to give the 1,7-bis(4-cyanomethylphenoxy)heptane as a tan solid, 2.58 g, 82%.

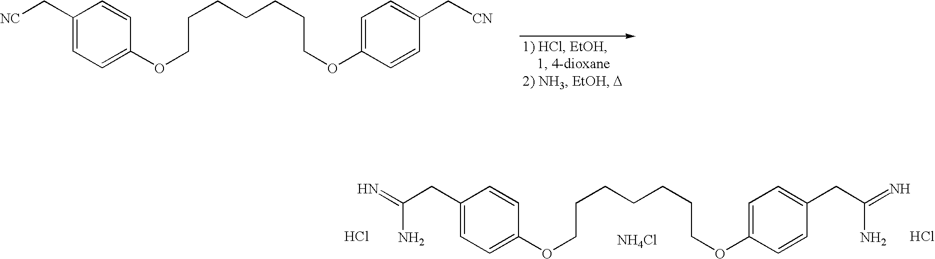

Step 2: A solution of 1,5-bis(4-cyanomethylphenoxy)heptane (750 mg, 5.07 mmol) in a mixture of 1,4-dioxane (10 mL) and absolute ethanol (10 mL) was saturated with HCl at 0° C. The mixture was then stirred at room temperature for the 3 days. The solvent was evaporated under reduced pressure and the residue was dried in vacuo. The residue was dissolved in 2 N ammonia in ethanol (20 mL) and the mixture was heated at reflux for 3 h. The solvent was evaporated under reduced pressure. The crude solid was recrystallized from 2 N HCl/acetone. The crystals were collected and dried in vacuo. The title compound was obtained as an off-white solid, 655.3 mg, 60%.

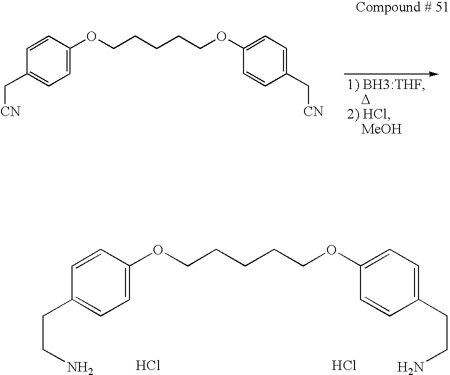

Step 1: A solution of borane:tetrahydrofuran complex (10 mL, 10 mmol) was added to a solution of the bis-nitrile (510 mg, 1.53 mmol) at 0° C. The mixture was then heated at reflux for 18 hours, then cooled with an ice bath. The excess of reagent was quenched by the slow addition of methanol (10 mL). The resulting mixture was heated at reflux for 15 minutes, then the solvent was removed under reduced pressure. The residue was coevapotated 3 times with methanol, then suspended in mixture of methanol (20 mL) and concentrated HCl (6 mL). The mixture was heated at reflux for 1.5 hour. The mixture was then reduce to about 5 mL under reduced pressure. A fine white solid had formed. The mixture was diluted with ethanol and cooled to −10° C. The solid was collected by filtration, rinsed with cold ethanol and dried overnight in vacuo. The 1,5-bis (4-(2-aminoethyl)phenoxy)pentane dihydrochloride was obtained as a fine white powder, 564.6 mg, 89%.

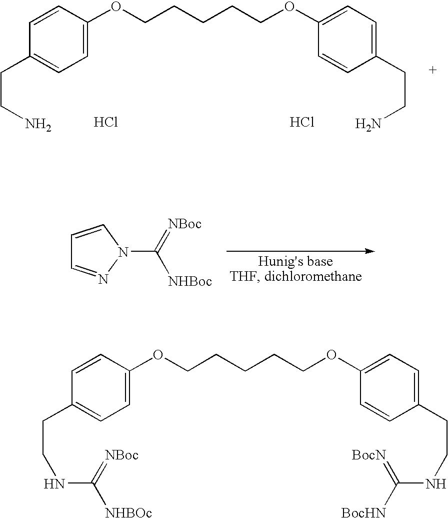

Step 2: N,N′-bis(tert-butoxycarbonyl)-1H-pyrazole-1-carboxamidine (0.78 g, 2.5 mmol) was added to a suspension of the 1,5-bis(4-(2-aminoethyl)phenoxy)pentane dihydrochloride (470 mg, 1.13 mmol) and Hunig's base (0.435 mL) in a mixture of THF (5 mL) and dichloromethane (20 mL). The mixture was stirred at room temperature for 2 days. Excess reagent was quenched with 1,2-ethylenediamine. The mixture was diluted with chloroform, washed subsequently with 1 N HCl, saturated sodium carbonate, brine, and dried over magnesium sulfate. The solvent was removed under reduced pressure. The crude product was purified by flash chromatography on silica gel (0.5% to 1% MeOH in CHCl₃) giving a white foamy solid 246.5 mg, 26%.

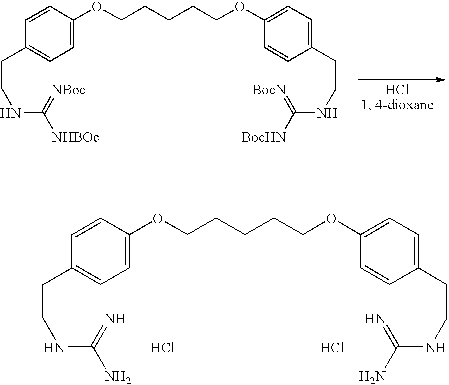

Step 3: A solution of 4 M HCl in 1,4-dioxane (5 mL) was added to a solution of the protected bis guanidino compound (246 mg, 0.297 mmol) in 1,4-dioxane (10 mL). The mixture was stirred at room temperature for one day. The solvent was evaporated under reduced pressure. The product was dissolved in water, then the aqueous soultion was lyophilized, giving the title compound as a white solid, 146.4 mg, 99%.

1. A method of treating or preventing an amyloid-related disease in a subject comprising administering to said subject a therapeutic amount of an amidine compound.

2. (canceled)

3. The method according to claim 1, wherein said compound is a bis(amidine) compound.

4. The method according to claim 1, wherein said compound is a bis(benzamidine) compound.

5. The method according to claim 1, wherein said compound is selected according to the following Formula: 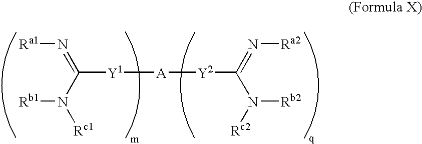
wherein each R^{a1}, R^{b1}, R^{c1}, R^{a2}, R^{b2}, and R^{c2} is independently a hydrogen, a Z group, or R^{a1} and R^{b1} or R^{a2} and R^{b2} are both taken together along with the nitrogen atoms to which they are bound to form a ring structure;
each of Y¹ and Y² is independently a direct bond or a linking moiety;
m and q are each independently an integer selected from zero to five inclusive, such that 2≦m+q≦5; and
A is a carrier moiety selected from substituted or unsubstituted aliphatic and aromatic groups, and combinations thereof; such that the Y¹ and Y² moieties are bonded to an aromatic group;
Z is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′, or (CR′R″)₀₋₁₀OR′ group, or the side chain of any naturally occurring amino acid;
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group;
and pharmaceutically acceptable salts thereof.

6. The method according to claim 1, wherein said compound is selected according to the following Formula: 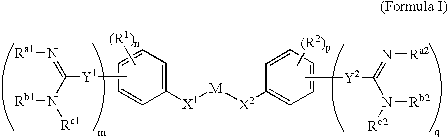
wherein each R^{a1}, R^{b1}, R^{c1}, R^{a2}, R^{b2}, and R^{c2} is independently a hydrogen, a Z group, or R^{a1} and R^{b1} or R^{a2} and R^{b2} are both taken together along with the nitrogen atoms to which they are bound to form a ring structure;
each of Y¹ and Y² is independently a direct bond or a linking moiety;
each of R¹ and R² is independently a hydrogen or a Z group, or two adjacent or proximate R¹ or R² groups taken together with the ring to which they are bound form a fused aromatic, heteroaromatic, cycloalkyl, or heterocylic structure;
each of X¹ and X² is independently an alkylene group, an oxygen, a NR′ group (where R′ is hydrogen, a C₁-C₅ alkyl, C₂-C₅ alkenyl, C₂-C₅ alkynyl, or aryl group), a sulfonamide group, a carbonyl, amide, C₁-C₅ alkylene group, C₂-C₅ alkenyl group, C₂-C₅ alkynyl group, or a sulfur atom, or combinations thereof or a direct bond;
M is an alkylene group, an alkenylene group, an alkynylene group, an alkoxyalkylene group, an alkylaminoalkylene group, a thioalkoxyalkylene group, an arylenedialkylene group, an alkylenediarylene group, a heteroarylenedialkylene group, an arylene group, a heteroarylene group, an oligoethereal or oligo(alkyleneoxide) group, or an arylene-di(oligoalkyleneoxide) group, each of which may be substituted or unsubstituted;
Z is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′, or (CR′R″)₀₋₁₀OR′ group, or the side chain of any naturally occurring amino acid;
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group;
m and q are each independently an integer selected from zero to four inclusive, and n and p are each independently an integer selected from zero to four inclusive, such that m+n=5 and p+q=5, wherein either m or q is at least one;
and pharmaceutically acceptable salts thereof.

7. The method according to claim 1, wherein said compound is selected according to the following Formula: 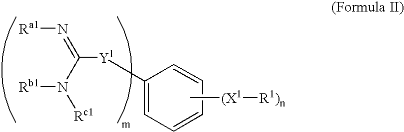
wherein each R^{a1}, R^{b1}, R^{c1}, R^{a2}, R^{b2}, and R^{c2} is independently a hydrogen, a Z group other than a substituted aryl group or a substituted alkyl group, or R^{a1} and R^{b1} or R^{a2} and R^{b2} are both taken together along with the nitrogen atoms to which they are bound to form a ring structure;
Y¹ is a direct bond or a linking moiety;
R¹ is a hydrogen or a Z group, or two adjacent or proximate R¹ groups taken together with the corresponding X¹ groups and the ring to which they are bound form a fused aromatic, heteroaromatic, cycloalkyl, or heterocylic structure;
X¹ is an alkylene group, an oxygen, a NR′ group (where R′ is hydrogen, a C₁-C₅ alkyl, C₂-C₅ alkenyl, C₂-C₅ alkynyl, or aryl group), a sulfonamide group, a carbonyl, amide, C₁-C₅ alkylene group, C₂-C₅ alkenyl group, C₂-C₅ alkynyl group, or a sulfur atom, or combinations thereof or a direct bond;
Z is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′, or (CR′R″)₀₋₁₀OR′ group, or the side chain of any naturally occurring amino acid;
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group;
m is an integer selected from one to six inclusive, and n is an integer selected from zero to five inclusive, such that m+n=6;
and pharmaceutically acceptable salts thereof.

8. The method according to claim 1, wherein said therapeutic compound is selected according to the following Formula: 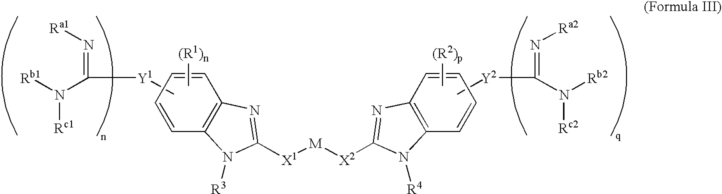
wherein each R^{a1}, R^{b1}, R^{c1}, R^{a2}, R^{b2}, and R^{c2} is independently a hydrogen, a Z group, or R^{a1} and R^{b1} or R^{a2} and R^{b2} are both taken together along with the nitrogen atoms to which they are bound to form a ring structure;
each of Y¹ and Y² is independently a direct bond or a linking moiety;
each of R¹ and R² is independently a hydrogen or a Z group, or two adjacent or proximate R¹ or R² groups taken together with the ring to which they are bound form a fused aromatic, heteroaromatic, cycloalkyl, or heterocylic structure;
each of R³ and R⁴ is independently selected from the group consisting of hydrogen, substituted or unsubstituted straight or branched alkyl, cycloalkyl, carbocyclic, aryl, heterocyclic, and heteroaryl;
each of X¹ and X² is independently an alkylene group, an oxygen, a NR′ group (where R′ is hydrogen, a C₁-C₅ alkyl, C₂-C₅ alkenyl, C₂-C₅ alkynyl, or aryl group), a sulfonamide group, a carbonyl, amide, C₁-C₅ alkylene group, C₂-C₅ alkenyl group, C₂-C₅ alkynyl group, or a sulfur atom, or combinations thereof or a direct bond;
M is an alkylene group, an alkenylene group, an alkynylene group, an alkoxyalkylene group, an alkylaminoalkylene group, a thioalkoxyalkylene group, an arylenedialkylene group, an alkylenediarylene group, a heteroarylenedialkylene group, an arylene group, a heteroarylene group, an oligoethereal or oligo(alkyleneoxide) group, or an arylene-di(oligoalkyleneoxide) group, each of which may be substituted or unsubstituted;
Z is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′, or (CR′R″)₀₋₁₀OR′ group, or the side chain of any naturally occurring amino acid;
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group;
m, n, p, and q are each independently an integer selected from zero to three inclusive, m+n≦4, p+q≦4, and m+q≧1;
and pharmaceutically acceptable salts thereof.

9. The method according to claim 1, wherein said compound is selected according to the following Formula: 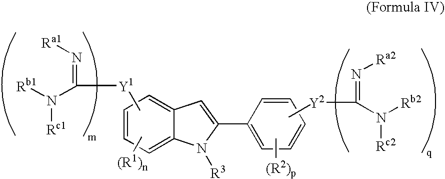
wherein each R^{a1}, R^{b1}, R^{c1}, R^{a2}, R^{b2}, and R^{c2} is independently a hydrogen, a Z group, or R^{a1} and R^{b1} or R^{a2} and R^{b2} are both taken together along with the nitrogen atoms to which they are bound to form a ring structure;
each of Y¹ and Y² is independently a direct bond or a linking moiety;
each of R¹ and R² is independently a hydrogen or a Z group, or two adjacent or proximate R¹ or R² groups taken together with the ring to which they are bound form a fused aromatic, heteroaromatic, cycloalkyl, or heterocylic structure;
R³ is selected from the group consisting of hydrogen, substituted or unsubstituted straight or branched alkyl, cycloalkyl, carbocyclic, aryl, heterocyclic, and heteroaryl;
Z is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′, or (CR′R″)₀₋₁₀OR′ group, or the side chain of any naturally occurring amino acid;
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group;
m and n are each independently an integer selected from zero to three inclusive, p and q are each independently an integer selected from zero to four inclusive, m+n≦4, p+q≦5, and m+q≧1;
and pharmaceutically acceptable salts thereof.

10. The method according to claim 1, wherein said compound is selected according to the following Formula: 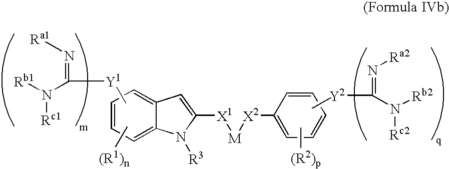
wherein each R^{a1}, R^{b1}, R^{c1}, R^{a2}, R^{b2}, and R^{c2} is independently a hydrogen, a Z group, or R^{a1} and R^{b1} or R^{a2} and R^{b2} are both taken together along with the nitrogen atoms to which they are bound to form a ring structure;
each of Y¹ and Y² is independently a direct bond or a linking moiety;
each of R¹ and R² is independently a hydrogen or a Z group, or two adjacent or proximate R¹ or R² groups taken together with the ring to which they are bound form a fused aromatic, heteroaromatic, cycloalkyl, or heterocylic structure;
R³ is selected from the group consisting of hydrogen, substituted or unsubstituted straight or branched alkyl, cycloalkyl, carbocyclic, aryl, heterocyclic, and heteroaryl;
each of X¹ and X² is independently an alkylene group, an oxygen, a NR′ group (where R′ is hydrogen, a C₁-C₅ alkyl, C₂-C₅ alkenyl, C₂-C₅ alkynyl, or aryl group), a sulfonamide group, a carbonyl, amide, C₁-C₅ alkylene group, C₂-C₅ alkenyl group, C₂-C₅ alkynyl group, or a sulfur atom, or combinations thereof or a direct bond;
M is an alkylene group, an alkenylene group, an alkynylene group, an alkoxyalkylene group, an alkylaminoalkylene group, a thioalkoxyalkylene group, an arylenedialkylene group, an alkylenediarylene group, a heteroarylenedialkylene group, an arylene group, a heteroarylene group, an oligoethereal or oligo(alkyleneoxide) group, or an arylene-di(oligoalkyleneoxide) group, each of which may be substituted or unsubstituted;
Z is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′, or (CR′R″)₀₋₁₀OR′ group, or the side chain of any naturally occurring amino acid;
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group;
m and n are each independently an integer selected from zero to three inclusive, p and q are each independently an integer selected from zero to four inclusive, m+n≦4, p+q≦5, and m+q≧1;
and pharmaceutically acceptable salts thereof.

11. The method according to claim 1, wherein said compound is selected according to the following Formula: 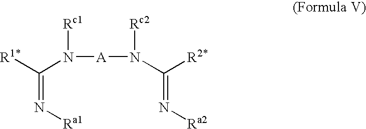
wherein each R^{a1}, R^{b1}, R^{c1}, R^{a2}, R^{b2}, and R^{c2} is independently a hydrogen, a Z group, or R^{a1} and R^{b1} or R^{a2} and R^{b2} are both taken together along with the nitrogen atoms to which they are bound to form a ring structure;
A is a carrier moiety selected from substituted or unsubstituted aliphatic and aromatic groups, and combinations thereof; such that the Y¹ and Y² moieties are bonded to an aromatic group;
Z is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′, or (CR′R″)₀₋₁₀OR′ group, or the side chain of any naturally occurring amino acid;
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group;
and pharmaceutically acceptable salts thereof.

12. The method according to claim 1, wherein said amyloid-related disease is associated with amyloid-β.

13. The method according to claim 1, wherein said amyloid-related disease is Alzheimer's disease, cerebral amyloid angiopathy, Down's syndrome, or inclusion body myositis.

14. The method according to claim 1, wherein said amyloid-related disease is type II diabetes.

15. The method according to claim 1, where said subject is a human.

16. The method according to claim 5, wherein said ring structure is selected from the following: 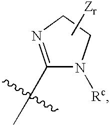
wherein r is an integer from zero to 4 inclusive, 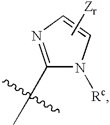
wherein r is an integer from zero to 2 inclusive, 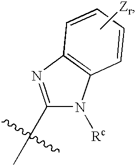
wherein r is an integer from zero to 4 inclusive, and
Z and R^{c} are each independently a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, —NO₂, halogen, (CR′R″)₀₋₁₀C(halogen)₃, (CR′R″)₁₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)O₀₋₁₀CO₂R′, or (CR′R″)O₀₋₁₀R′ group, or the side chain of any naturally occurring amino acid; and
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

17. The method according to claim 5, wherein each of said R^{a1}, R^{b1}, R^{d1}, R^{a2}, R^{b2}, and R^{c2} groups is a hydrogen, hydroxy group, a substituted or unsubstituted C₁-C₈ alkyl or C₁-C₈ alkoxy group.

18. The method according to claim 5, wherein each of said R^{a1}, R^{b1}, R^{d1}, R^{a2}, R^{b2}, and R^{c2} groups is an aromatic group or heteroaromatic group.

19. The method according to claim 5, wherein each of said R^{a1}, R^{b1}, R^{c1}, R^{a2}, R^{b2}, and R^{c2} groups is a hydrogen, substituted or unsubstituted straight or branched alkyl, cycloalkyl, carbocyclic, aryl, heterocyclic, or heteroaryl group.

20. The method according to claim 5, wherein each of said Y¹ and Y² groups is a linking moiety of less than about 75 molecular weight.

21. The method according to claim 5, wherein said Y¹ and Y² groups is a direct bond.

22. The method according to claim 6, wherein each of said R¹ and R² groups is independently a hydrogen, a substituted or unsubstituted C₁-C₈ alkyl group, a substituted or unsubstituted C₁-C₈ alkenyl group, a halogen, a substituted or unsubstituted aryl or heteroaryl group, a substituted or unsubstituted amino group, a nitro group, or a substituted or unsubstituted C₁-C₈ alkoxy group.

23. The method according to claim 6, wherein said M group is —[(CH₂)ₛO]ₜ(CH₂)ₛ—, where t is 1 to 6 and s is 2 to 6.

24. The method according to claim 6, wherein said M group is a phenylenedialkylene group.

25. The method according to claim 6, wherein said M arylenedialkylene group is 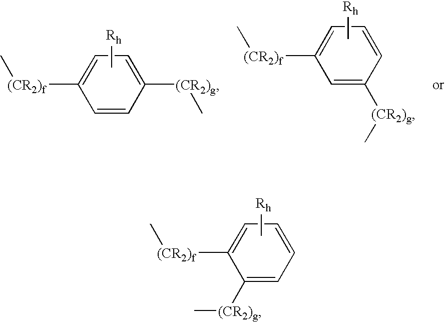
wherein each R group is independently a hydrogen or is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀O(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′, or (CR′R″)O₀₋₁₀R′ group, or the side chain of any naturally occurring amino acid; and
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group; and 1≦f≦8, 1≦g≦8, 0≦h≦4.

26. The method according to claim 6, wherein said M group is a substituted or unsubstituted C₂-C₈ alkylene group, a substituted or unsubstituted C₁-C₈ alkenylene group, a substituted or unsubstituted C₂-C₈ alkynylene group.

27. The method according to claim 6, wherein said M group is 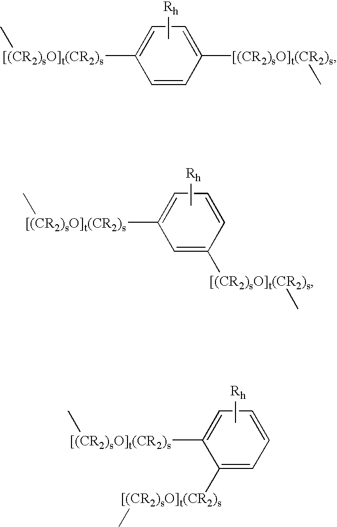
wherein 1≦t≦6, 0≦s≦6, 0≦h≦4, and each R group is independently a hydrogen or is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R, or (CR′R″)₀₋₁₀OR′ group, or the side chain of any naturally occurring amino acid; and
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group.

28. (canceled)

29. (canceled)

30. (canceled)

31. The method according to claim 6, wherein said M group is 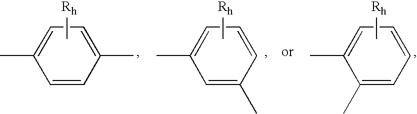
wherein each R group is independently a hydrogen or is a substituted or unsubstituted moiety selected from straight or branched alkyl, cycloalkyl, alkoxy, thioalkyl, alkenyl, alkynyl, heterocyclic, carbocyclic, aryl, aryloxy, aralkyl, aryloxyalkyl, arylacetamidoyl, alkylaryl, heteroaralkyl, alkylcarbonyl, arylcarbonyl, heteroarylcarbonyl, or heteroaryl group, (CR′R″)₀₋₁₀NR′R″, (CR′R″)₀₋₁₀CN, NO₂, halogen, (CR′R″)₀₋₁₀C(halogen)₃, (CR′R″)₀₋₁₀CH(halogen)₂, (CR′R″)₀₋₁₀CH₂(halogen), (CR′R″)₀₋₁₀CONR′R″, (CR′R″)₀₋₁₀O(CNH)NR′R″, (CR′R″)₀₋₁₀S(O)₁₋₂NR′R″, (CR′R″)₀₋₁₀CHO, (CR′R″)₀₋₁₀O(CR′R″)₀₋₁₀H, (CR′R″)₀₋₁₀S(O)₀₋₃R′, (CR′R″)₀₋₁₀O(CR′R″)₀₋₁₀H, (CR′R″)₀₋₁₀S(CR′R″)₀₋₃H, (CR′R″)₀₋₁₀OH, (CR′R″)₀₋₁₀COR′, (CR′R″)₀₋₁₀(substituted or unsubstituted phenyl), (CR′R″)₀₋₁₀(C₃-C₈ cycloalkyl), (CR′R″)₀₋₁₀CO₂R′, or (CR′R″)₀₋₁₀OR′ group, or the side chain of any naturally occurring amino acid; and
R′ and R″ are each independently hydrogen, a C₁-C₅ alkyl, C₂-C₅ alkenyl, C₂-C₅ alkynyl, or aryl group, or R′ and R″ taken together are a benzylidene group or a —(CH₂)₂O(CH₂)₂— group; and 0≦h≦4.

32. (canceled)

33. (canceled)

34. (canceled)

35. (canceled)

36. (canceled)

37. The method according to claim 2, wherein m=1, n=0, 1, or 2, p=0, 1, or 2, and q=1.

38. The method according to claims 5, wherein R^{a1}=R^{a2}, R^{b1}=R^{b2}, R^{c1}=R^{c2}, m=q, n=p, and Y¹=Y₂.

39. The method according to claim 6, wherein R¹=R², and X¹=X².

40. The method according to claim 5, wherein said pharmaceutically acceptable salt is a hydrohalide salt or a 2-hydroxyethanesulfonate salt.

41. (canceled)

42. A pharmaceutical composition for b treating or preventing an amyloid-related disease comprising a compound according to claim 5.

43. The method according to claim 5, wherein said linking moiety is —(CH₂)ₙ— (wherein n is 1, 2, or 3), —NR³—, —NH—, —S—, —O—, —NH—CH₂—, or —CH═CH—, or combinations thereof, wherein R3 is selected from the group consisting of hydrogen, substituted or unsubstituted straight or branched alkyl, cycloalkyl, carbocyclic, aryl, heterocyclic, and heteroaryl.

44. A chemical compound according to the formula: 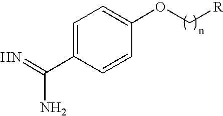
wherein n is an integer from 7 to 10, and R is Br or CO₂H, and pharmaceutically acceptable salts thereof.

45. (canceled)

46. A pharmaceutical composition for treating or preventing an amyloid-related disease comprising a therapeutically effective amount of a chemical compound according to claim 44.

47. The method of claim 1, wherein said amidine compound causes in an Alzheimer's patient a stabilization of cognitive function, prevention of a further decrease in cognitive function, or prevention, slowing, or stopping of disease progression.

48. The method according to claim 5, wherein Z is a substituted or unsubstituted moiety selected from straight or branched C₁-C₅ alkyl, C₃-C₈ cycloalkyl, C₁-C₆ alkoxy, C₁-C₆ thioalkyl, C₂-C₆ alkenyl, C₂-C₆ alkynyl, heterocyclic, carbocyclic, phenyl, phenoxy, benzyl, phenyloxyalkyl, arylacetamidoyl, alkylaryl, heteroaralkyl, alkylcarbonyl, arylcarbonyl, heteroarylcarbonyl, or heteroaryl group, —NH₂, —CN, NO₂, F, Cl, Br, I, —CF₃, (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, —SO₃H, —CH₂OCH₃, —OCH₃, —SH, —SCH₃, —OH, (CR′R″)₀₋₃COR′, (CR′R″)₀₋₃(substituted or unsubstituted phenyl), (CR′R″)₀₋₃(C₃-C₈ cycloalkyl), —CO₂H, or (CR′R″)₀₋₃OR′ group.

49. The method according to claim 1, wherein said compound is selected from the group consisting of 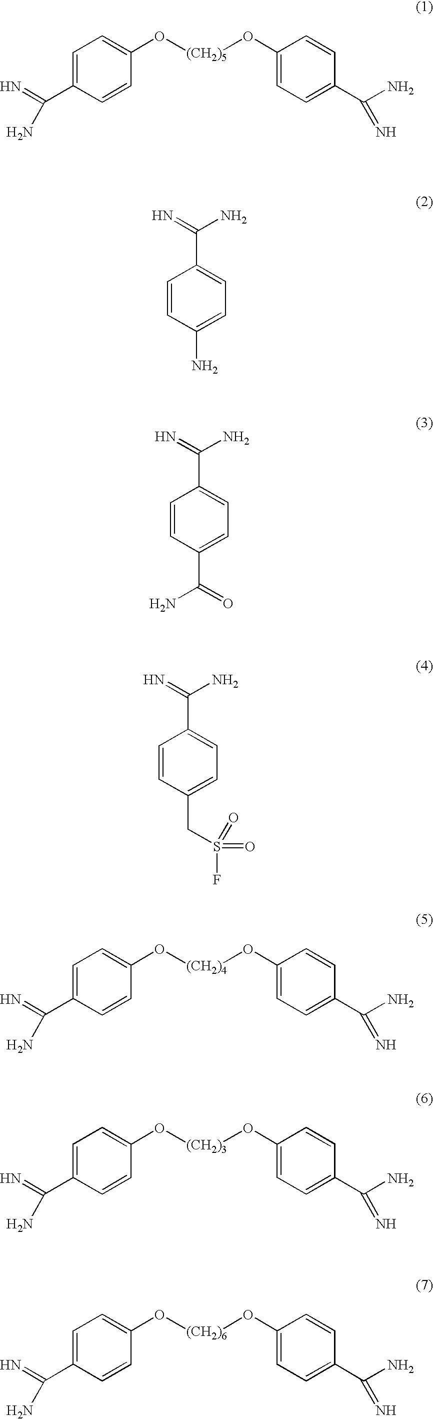 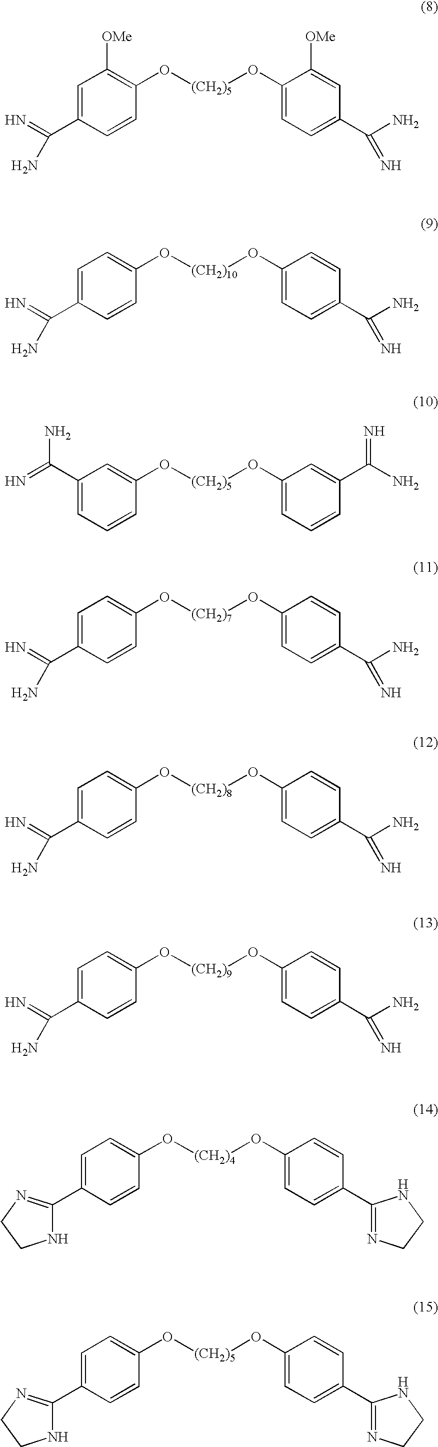 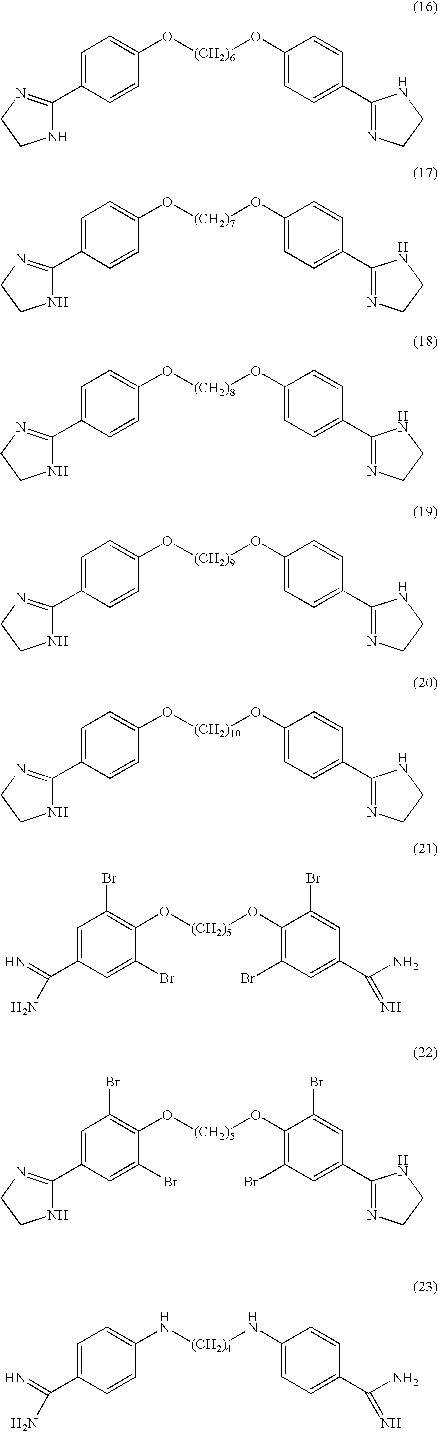 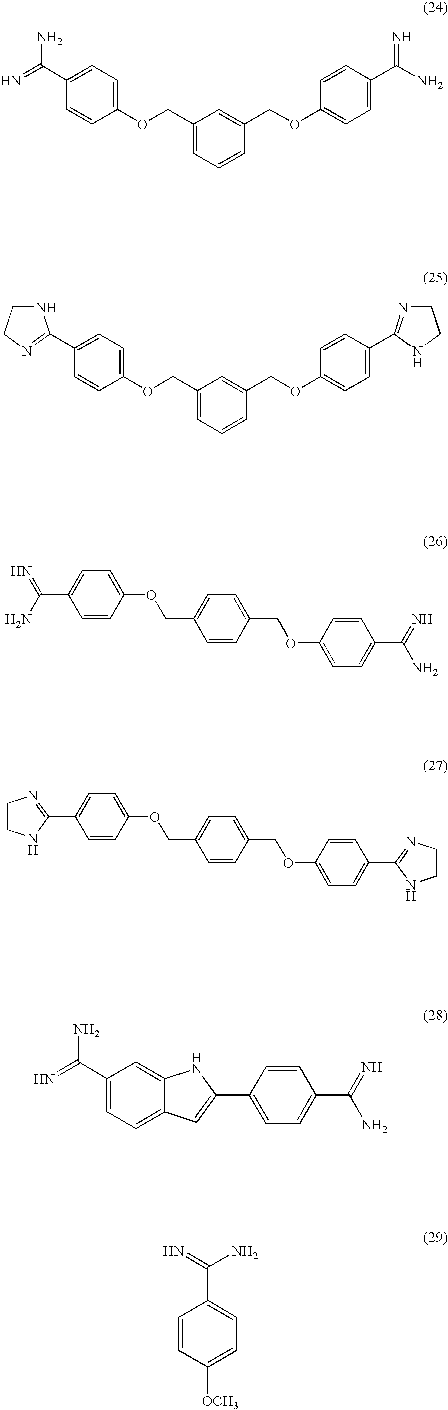
and pharmaceutically acceptable salts thereof.

50. The method according to claim 1, wherein said compound is selected from the group consisting of 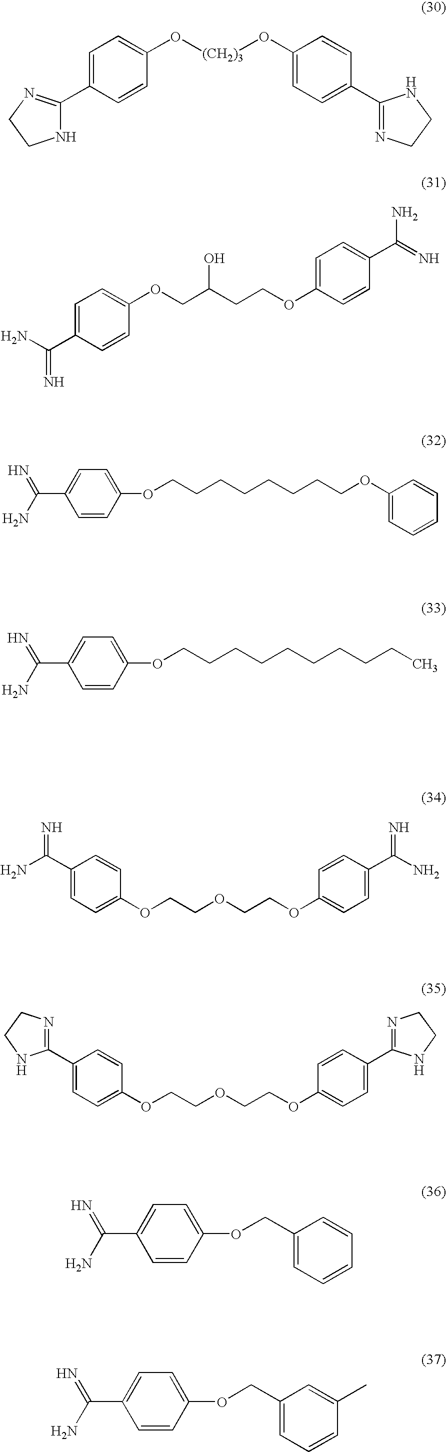 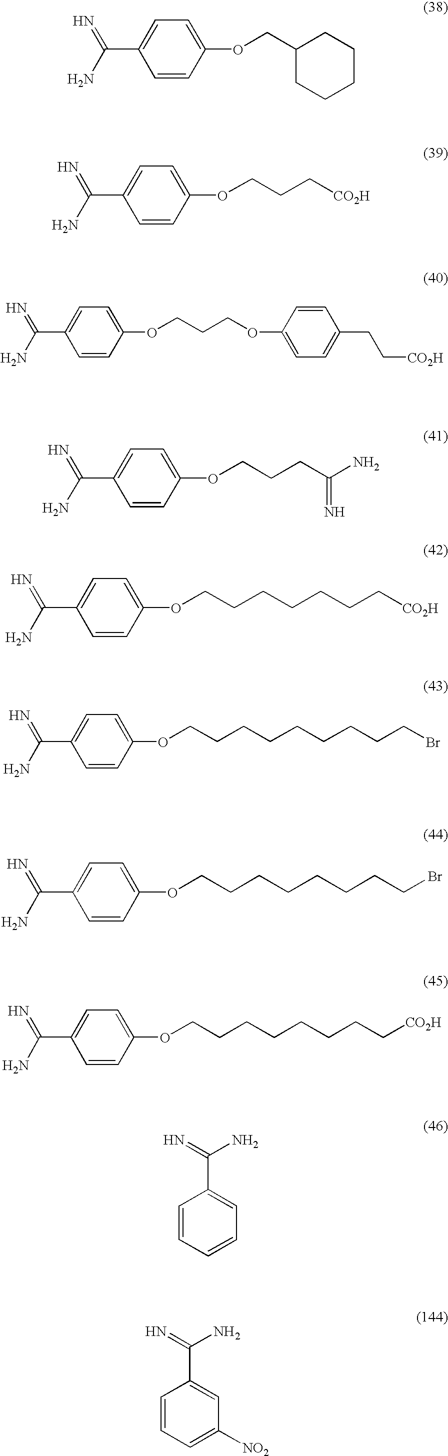 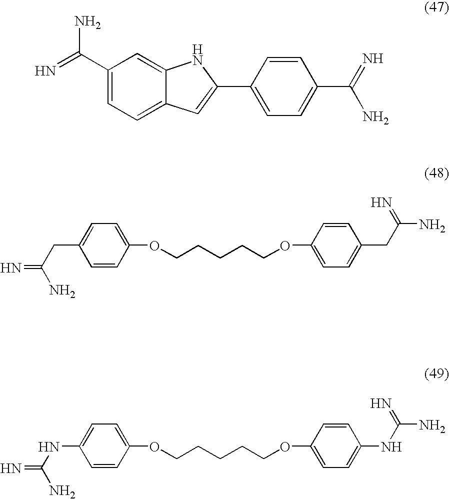
and pharmaceutically acceptable salts thereof.

51. The method according to claim 1, wherein said compound is selected from the group consisting of 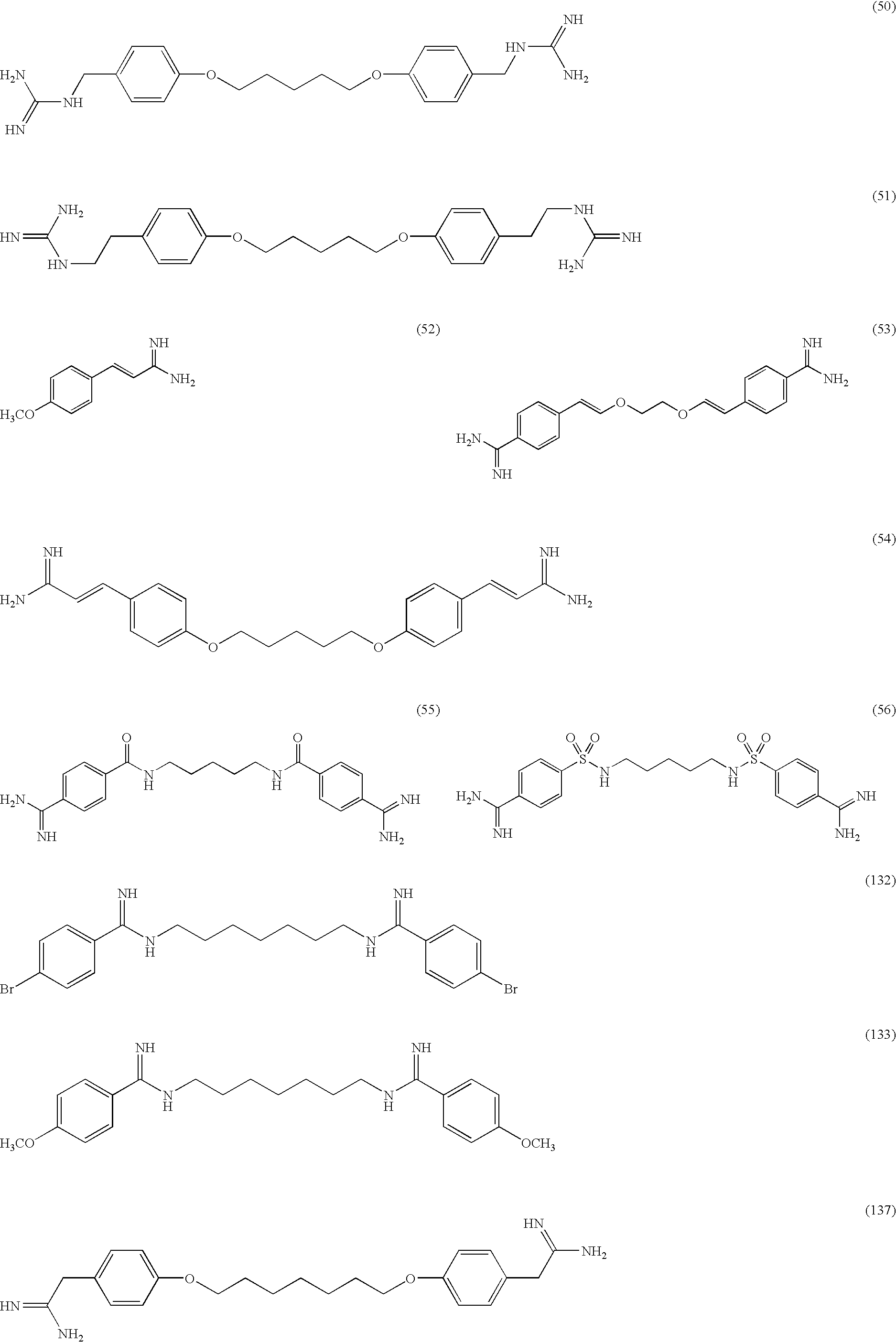 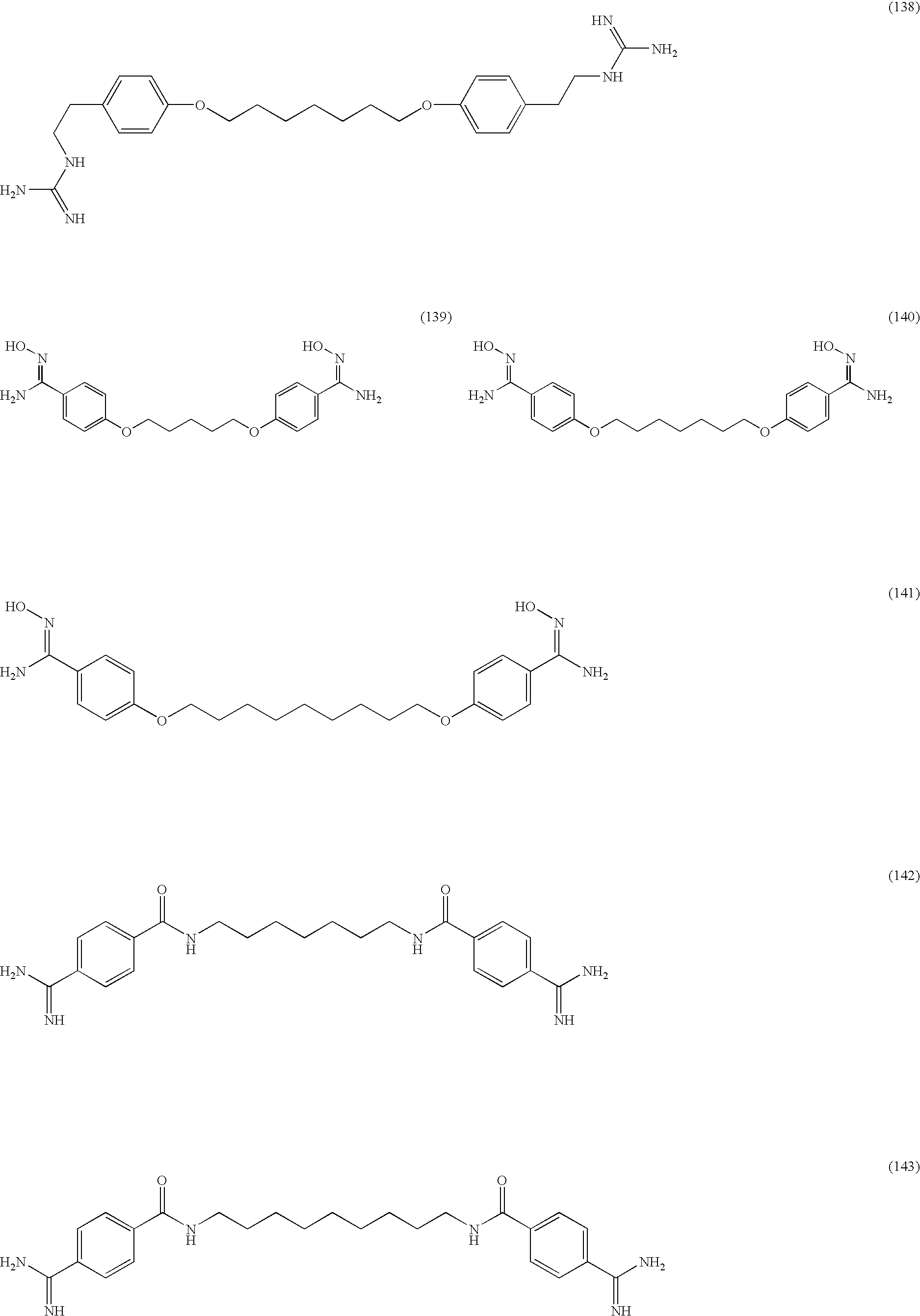
and pharmaceutically acceptable salts thereof.

52. The method according to claim 1, wherein said compound is selected from the group consisting of 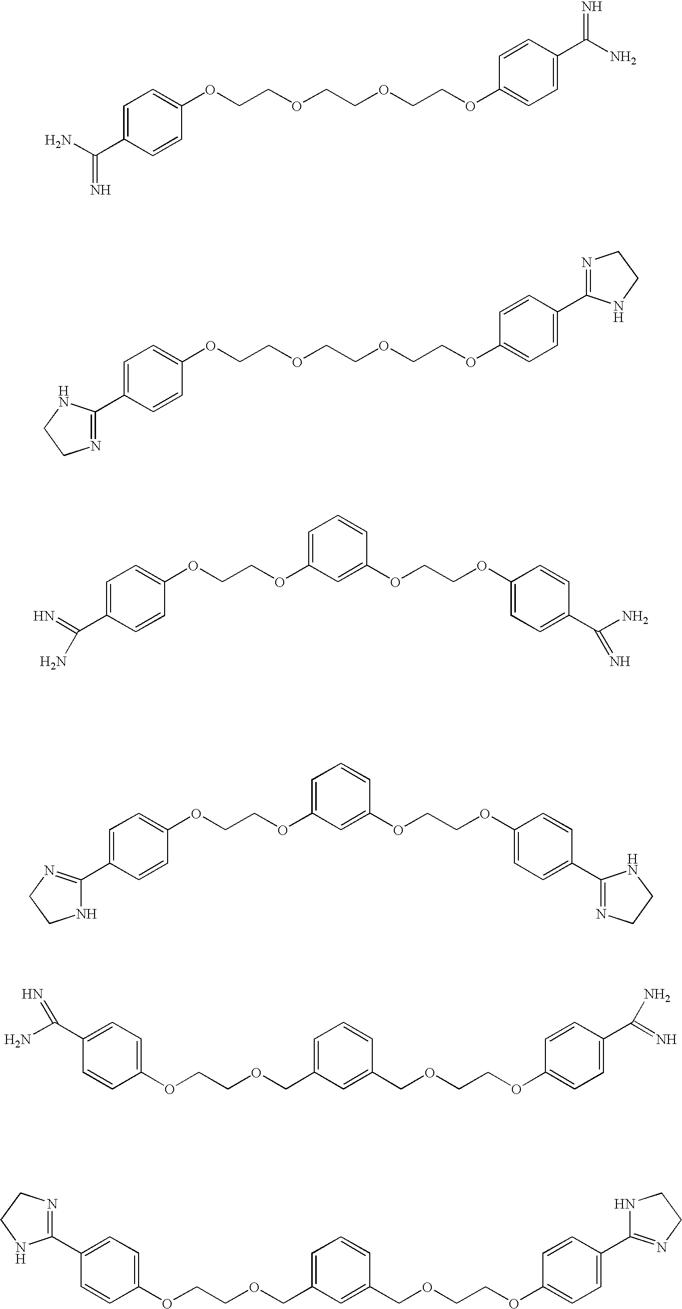
and pharmaceutically acceptable salts thereof.

53. The method according to claim 1, wherein said compound is selected from the group consisting of 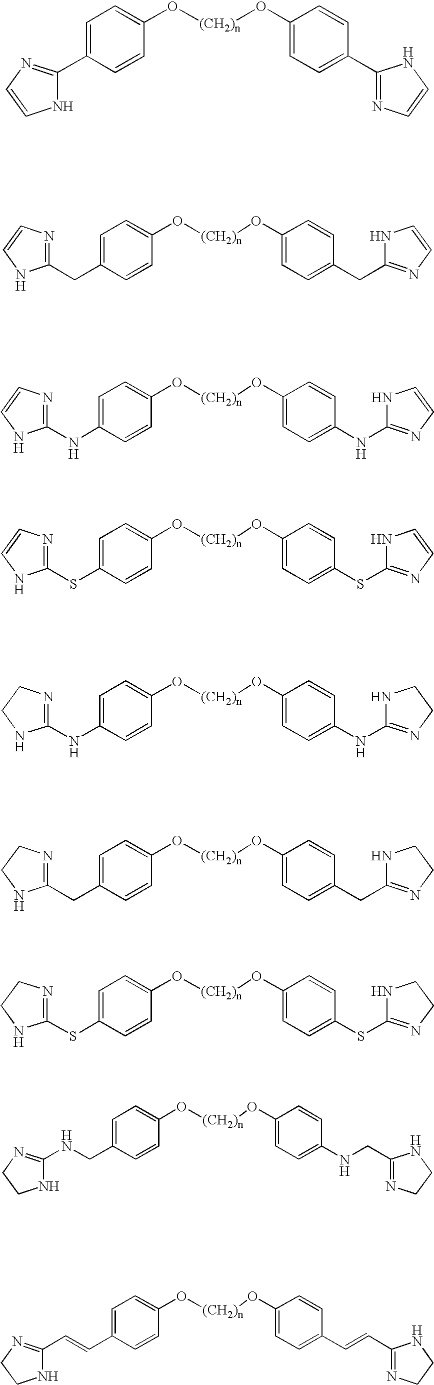 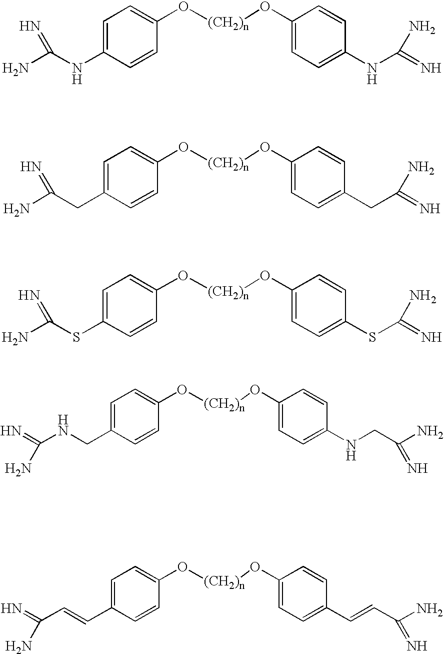
wherein n is an integer from 1 to 12,
and pharmaceutically acceptable salts thereof.

54. The method according to claim 1, wherein said compound is selected from the group consisting of 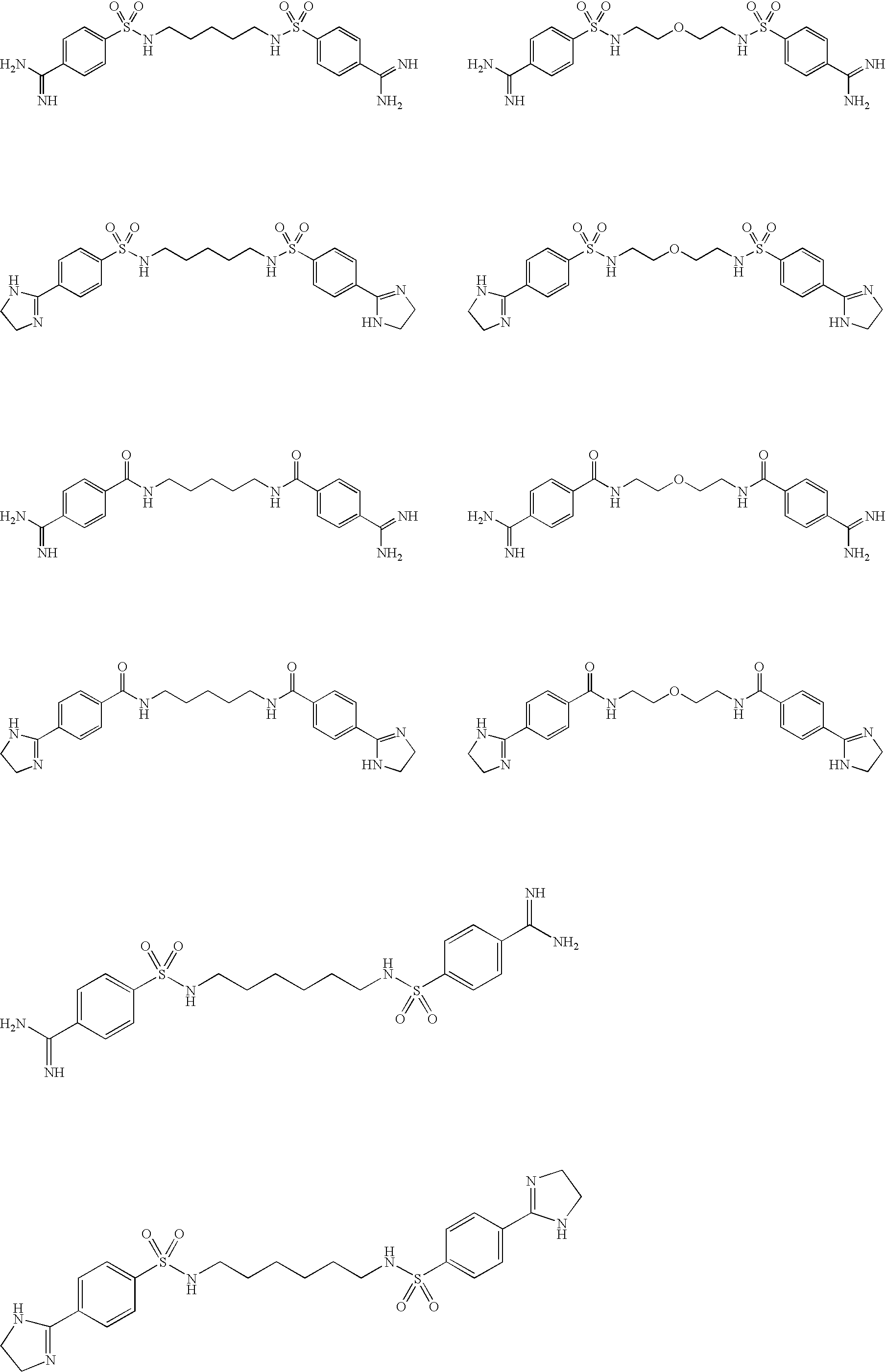 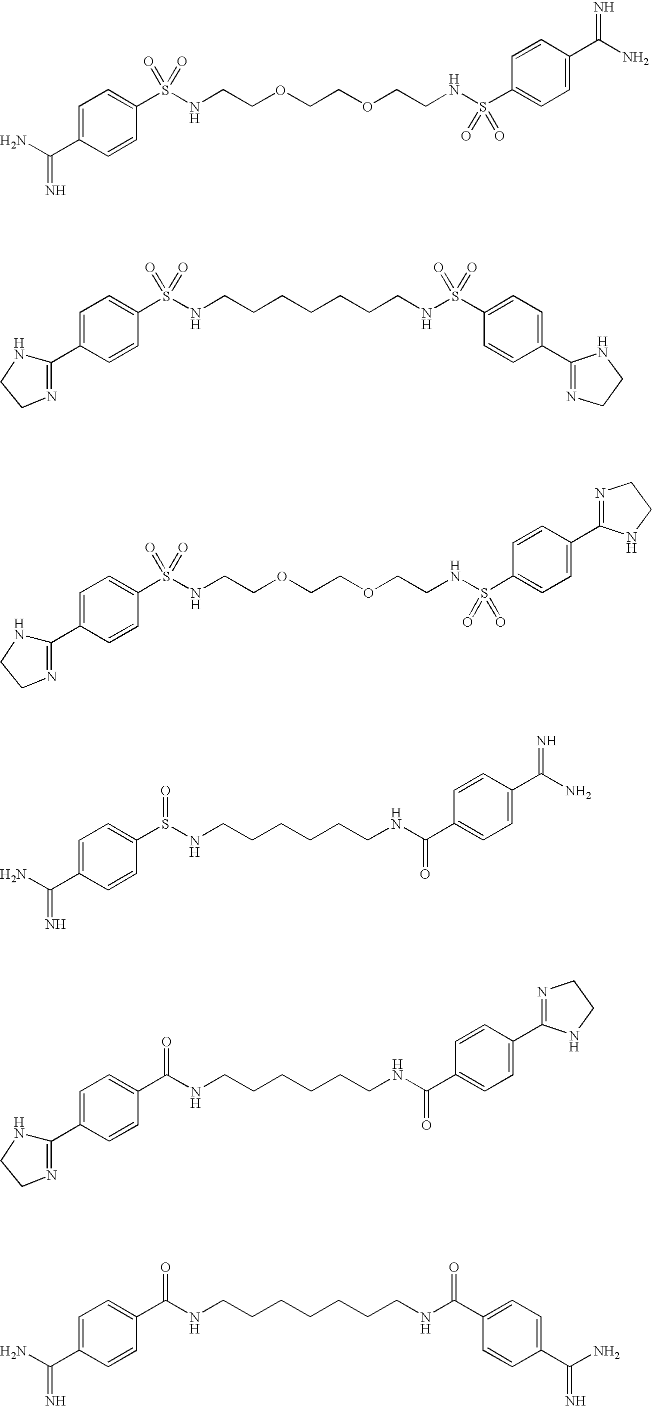 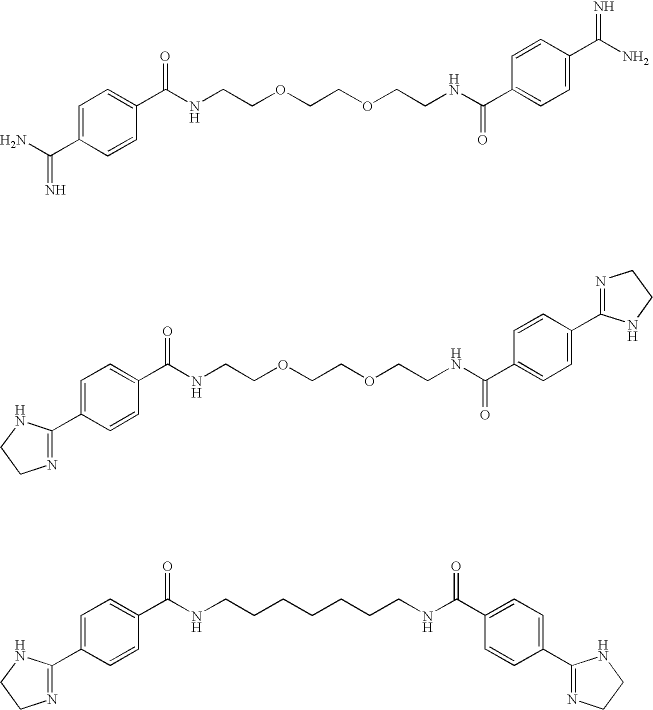
and pharmaceutically acceptable salts thereof.

55. The method according to claim 1, wherein said compound is used therapeutically or prophylactically to treat a human in need thereof.

56. The method according to claim 5, wherein said compound reduces or inhibits amyloid fibril formation or deposition, neurodegeneration, or cellular toxicity.

57. A chemical compound according to claim 44, having the following structure: 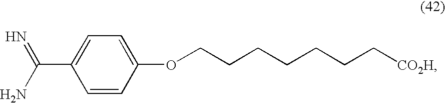
and pharmaceutically acceptable salts thereof.

58. A chemical compound according to claim 44, having the following structure: 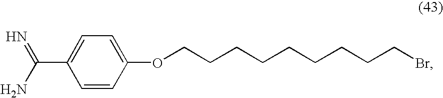
and pharmaceutically acceptable salts thereof.

59. A chemical compound according to claim 44, having the following structure: 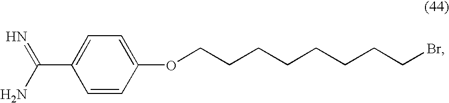
and pharmaceutically acceptable salts thereof.

60. A chemical compound according to claim 44 having the following structure: 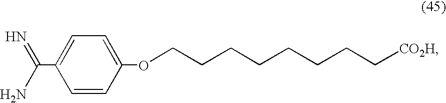
and pharmaceutically acceptable salts thereof.

61. (canceled)
